SEQUENCE LISTING (71 sequences)

SEQ ID NO: 1 - protein, 511 aa, Vesiculovirus indiana
  source 1..511: note = G [Vesicular stomatitis Indiana virus]. Accession No QQL06413
MKCLLYLAFLFIGVNCKFTIVFPHNQKGTWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNDICPTVHNSTTWHSDYKVKGLCDSNLISTDITFFSEDGELSSLGKGGTGFRSNHFAYETGDKACKMQYCRHWGVRLPSGVWFEMDDKDLFAAARFPECPEGSSISAPSQTSVDVSLIQDVERILDYSLCQETWSKIRAGLPISPVDLSYLAPKNPGTGPAFTIINGTLKYFETRYIRVDIVAPILSRMVGMISGTTTERELWDDWAPYEDVEIGPNGVLRTSSGYKFPLYMIGHGMLDSDLHLSSKAQVFEHPHIQDAASQLPDDETLFFGDTGLSKNPIELVEGWFSGWKSSIASFFFIIGLIIGLFLVLRVGIYLCIKLKHTKKRQIYTDIEMNRLGK

SEQ ID NO: 2 - protein, 25 aa, Vesiculovirus indiana
  source 1..25: note = Vesicular stomatitis Virus G protein (VSV-G) transmembrane domain
IASFFFIIGLIIGLFLVLRVGIHLC

SEQ ID NO: 3 - protein, 27 aa, Vesiculovirus indiana
  source 1..27: note = Vesicular stomatitis Virus G protein (VSV-G) transmembrane domain
SSIASFFFIIGLIIGLFLVLRVGIHLC

SEQ ID NO: 4 - protein, 24 aa, synthetic construct
  source 1..24: note = VSV-G TM variant
IASFFFIIGSSIGLALVLAGIHLC

SEQ ID NO: 5 - protein, 26 aa, synthetic construct
SSIASFFFIIGSSIGLALVLAGIHLC

SEQ ID NO: 6 - protein, 24 aa, synthetic construct
  source 1..24: note = VSV-G TM variant
IASFFFIIGSSIGLFLVLAGIHLC

SEQ ID NO: 7 - protein, 25 aa, synthetic construct
  source 1..25: note = VSV-G TM variant
IASFFFIIGSSIGLALVLRVGIHLC

SEQ ID NO: 8 - protein, 25 aa, synthetic construct
  source 1..25: note = VSV-G TM variant
IASFFFIIGSSIGLALVLRAGIHLC

SEQ ID NO: 9 - protein, 21 aa, synthetic construct
  source 1..21: note = VSV-G TM variant-deletion
SSIASFFFIIFLVLRVGIHLC

SEQ ID NO: 10 - protein, 27 aa, synthetic construct
  source 1..27: note = VSV-G TM variant
SSIASFFFIIALIIALFLVLRVGIHLC

SEQ ID NO: 11 - protein, 27 aa, synthetic construct
  source 1..27: note = VSV-G TM variant
SSIASFFFIILLIILLFLVLRVGIHLC

SEQ ID NO: 12 - protein, 27 aa, synthetic construct
  source 1..27: note = VSV-G TM variant
SSIASFFFIIALIIGLFLVLRVGIHLC

SEQ ID NO: 13 - protein, 27 aa, synthetic construct
  source 1..27: note = VSV-G TM variant
SSIASFFFIIGLIIALFLVLRVGIHLC

SEQ ID NO: 14 - protein, 25 aa, synthetic construct
  source 1..25: note = VSV-G TM variant
IASFFFIIGSSIGLALVLRAGIHLC

SEQ ID NO: 15 - protein, 24 aa, synthetic construct
  source 1..24: note = Influenza hemagglutinin A chain Transmembrane domain
ILSIYSTVASSLALAIMVAGLSLW

SEQ ID NO: 16 - protein, 22 aa, synthetic construct
  source 1..22: note = VAMP 2 transmembrane domain
MMIILGVICAIILIIIIVYFST

SEQ ID NO: 17 - protein, 22 aa, synthetic construct
  source 1..22: note = Syntaxin transmembrane domain
IMIICCVILGIIIASTIGGIFG

SEQ ID NO: 18 - protein, 550 aa, Alphainfluenzavirus influenzae
  source 1..550: note = Influenza hemagglutinin A chain HEMA_I75A2 Accession No P43257
QDLPGNDNSTATLCLGHHAVPNGTIVKTITDDQIEVTNATELVQSSSTGKICNNPHKILDGRDCTLIDALLGDPHCDVFQDETWDLFVERGNAFSSCYPYDVPDYASLRSLVASSGTLEFITEGFTWTGVTQNGGSSACKRGPASGFFSRLNWLTKSGSTYPVLNVTMPNNDNFDKLYIWGVHHPSTNQEQTNLYVQASGRVTVSTRRSQQTIIPNIGSRPWVRGQSGRISIYWTIVKPGDVLVINSNGNLIAPRGYFKMRTGKSSIMRSDAPIDTCVSECITPNGSIPNDKPFQNVNKITYGACPKYVKQNSLKLATGMRNVPEKQTRGLFGAIAGFIENGWEGMIDGWYGFRHQNSEGTGQAADLKSTQAAIDQINGKLNRVIKKTNEKFHQIEKEFSEVEGRIQDLEKYVEDTKIDLWSYNADVLVALENQHTIDLTDSEMNKLFEKTRRQLRENAEDMGNGCFKIYHKCDNACIESIRNGTYDHDIYRDEALNNRFQIKGVELKSGYKDWILWISFAISCFLLCVVLLGFIMWACQRGNIRCNICI

SEQ ID NO: 19 - protein, 575 aa, Alphainfluenzavirus influenzae
  source 1..575: note = Influenza hemagglutinin A chain HA Accession No ABQ09853.1
MEKIVLLFAIVSLVKSDQICIGYHANNSTEQVDTIMEKNVTVTHAQDILEKTHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSAACPYQGKSSFFRNVVWLIKKNSTYPTIKRSYNNTNQEDLLVMWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICIKFVSSDD

SEQ ID NO: 20 - protein, 288 aa, synthetic construct
  source 1..288: note = Syntaxin1A, Accession no. NM_053788
MKDRTQELRTAKDSDDDDDVTVTVDRDRFMDEFFEQVEEIRGFIDKIAENVEEVKRKHSAILASPNPDEKTKEELEELMSDIKKTANKVRSKLKSIEQSIEQEEGLNRSSADLRIRKTQHSTLSRKFVEVMSEYNATQSDYRERCKGRIQRQLEITGRTTTSEELEDMLESGNPAIFASGIIMDSSISKQALSEIETRHSEIIKLENSIRELHDMFMDMAMLVESQGEMIDRIEYNVEHAVDYVERAVSDTKKAVKYQSKARRKKIMIIICCVILGIIIASTIGGIFG

SEQ ID NO: 21 - protein, 116 aa, synthetic construct
  source 1..116: note = VAMP2, Accession No. AAH55105
MSATAATVPPAAPAGEGGPPAPPPNLTSNRRLQQTQAQVDEVVDIMRVNVDKVLERDQKLSELDDRADALQAGASQFETSAAKLKRKYWWKNLKMMIILGVICAIILIIIIVYFST

SEQ ID NO: 22 - protein, 512 aa, synthetic construct
  source 1..512: note = Cocal Envelope Amino Acid
MNFLLLTFIVLPLCSHAKFSIVFPQSQKGNWKNVPSSYHYCPSSSDQNWHNDLLGITMKVKMPKTHKAIQADGWMCHAAKWITTCDFRWYGPKYITHSIHSIQPTSEQCKESIKQTKQGTWMSPGFPPQNCGYATVTDSVAVVVQATPHHVLVDEYTGEWIDSQFPNGKCETEECETVHNSTVWYSDYKVTGLCDATLVDTEITFFSEDGKKESIGKPNTGYRSNYFAYEKGDKVCKMNYCKHAGVRLPSGVWFEFVDQDVYAAAKLPECPVGATISAPTQTSVDVSLILDVERILDYSLCQETWSKIRSKQPVSPVDLSYLAPKNPGTGPAFTIINGTLKYFETRYIRIDIDNPIISKMVGKISGSQTERELWTEWFPYEGVEIGPNGILKTPTGYKFPLFMIGHGMLDSDLHKTSQAEVFEHPHLAEAPKQLPEEETLFFGDTGISKNPVELIEGWFSSWKSTVVTFFFAIGVFILLYVVARIVIAVRYRYQGSNNKRIYNDIEMSRFRK

SEQ ID NO: 23 - DNA, 1539 bp, synthetic construct
  source 1..1539: mol_type = other DNA; note = Cocal Envelope Nucleic Acid
atgaacttcctcctgctgacttttatcgtgctgcctctctgctcccacgccaagttctcgattgtgttcccccaatcccaaaaggggaactggaagaatgtgccctcctcgtaccactactgcccgtcctcctccgaccaaaactggcacaacgatctgctcggaatcaccatgaaggtcaagatgcccaagacccataaggctattcaggccgacggctggatgtgccacgccgcgaagtggatcaccacctgtgacttccggtggtacggtccgaagtacatcactcactcgattcactcaattcagccgactagcgagcagtgcaaagagagcatcaagcagacgaagcagggcacatggatgtcccccggattccctccccaaaactgcggatatgcgaccgtgaccgatagcgtggccgtggtggtgcaggccacccctcatcatgtgcttgtggatgagtacaccggagaatggatcgacagccagttcccgaacggaaaatgcgaaaccgaggagtgcgagactgtccacaactccactgtgtggtactccgactacaaggtcacgggcttgtgcgacgcgactttggtggacaccgaaatcaccttctttagcgaggatggaaagaaggagtccatcggcaaaccgaacactggttaccgctccaattacttcgcgtacgaaaagggagacaaagtctgcaagatgaattactgcaagcacgccggtgtcaggctgccatcaggagtgtggttcgaattcgtggaccaggacgtgtacgctgccgcgaagcttccggaatgtccagtcggggcaaccatttccgcaccgactcagacctctgtggatgtgtccctgatcctggacgtcgagagaatcctggactacagcctgtgtcaggagacttggtcgaagattcgctccaagcagcccgtgtcacctgtggatctgtcgtatctggccccgaagaaccctggtaccggcccagcctttaccatcataaacgggaccctgaagtacttcgaaactcggtatattcggattgacatcgacaaccccatcatctcgaaaatggtcggaaagatcagcggatcccagacagaaagggaactctggaccgaatggttcccgtacgagggcgtggaaatcggtccgaacgggatcctgaaaactcctacgggctacaagttccccctcttcatgattgggcatggcatgctggactccgatctccacaagacctcccaagctgaagtgttcgagcaccctcacctggccgaagcacccaagcagctgccagaggaagaaaccctcttcttcggggacaccggaatctcgaagaacccggtggaactgattgagggctggttctcatcatggaagtccaccgtggtcaccttcttcttcgccatcggagtgtttatcctgctttacgtggtggcccgcatcgtgattgccgtgcggtacagataccagggctccaacaacaagcgcatctacaacgatatcgagatgagccggttccgcaagtaa

SEQ ID NO: 24 - protein, 4 aa, synthetic construct
  source 1..4: note = linker
GGSG

SEQ ID NO: 25 - protein, 5 aa, synthetic construct
  source 1..5: note = linker
GGSGG

SEQ ID NO: 26 - protein, 5 aa, synthetic construct
  source 1..5: note = linker
GSGSG

SEQ ID NO: 27 - protein, 5 aa, synthetic construct
  source 1..5: note = linker
GSGGG

SEQ ID NO: 28 - protein, 5 aa, synthetic construct
  source 1..5: note = linker
GGGSG

SEQ ID NO: 29 - protein, 5 aa, synthetic construct
  source 1..5: note = linker
GSSSG

SEQ ID NO: 30 - protein, 5 aa, synthetic construct
  source 1..5: note = linker
GGGGS

SEQ ID NO: 31 - protein, 15 aa, synthetic construct
  source 1..15: note = linker
GGGGSGGGGSGGGGS

SEQ ID NO: 32 - protein, 45 aa, synthetic construct
  source 1..45: note = linker
GGTGGCGGTGGCTCGGGCGGTGGTGGGTCGGGTGGCGGCGGATCT

SEQ ID NO: 33 - protein, 24 aa, synthetic construct
  source 1..24: note = CD8 alpha transmembrane domain amino acid sequence
IYIWAPLAGTCGVLLLSLVITLYC

SEQ ID NO: 34 - DNA, 72 bp, synthetic construct
  source 1..72: mol_type = other DNA; note = CD8 alpha transmembrane domain nucleic acid sequence
atctacatctgggcgcccttggccgggacttgtggggtccttctcctgtcactggttatcaccctttactgc

SEQ ID NO: 35 - protein, 45 aa, synthetic construct
  source 1..45: note = CD8 alpha hinge domain amino acid sequence
TTTPAPRPPTPAPTIASQPLSLRPEACRPAAGGAVHTRGLDFACD

SEQ ID NO: 36 - DNA, 135 bp, synthetic construct
  source 1..135: mol_type = other DNA; note = CD8 alpha hinge domain nucleic acid sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 37 - protein, 27 aa, synthetic construct
  source 1..27: note = CD28 transmembrane domain amino acid sequence
FWVLVVVGGVLACYSLLVTVAFIIFWV

SEQ ID NO: 38 - DNA, 81 bp, synthetic construct
  source 1..81: mol_type = other DNA; note = CD28 transmembrane domain nucleic acid sequence
ttttgggtgctggtggtggttggtggagtcctggcttgctatagcttgctagtaacagtggcctttattattttctgggtg

SEQ ID NO: 39 - protein, 5 aa, synthetic construct
  source 1..5: note = hinge
DKTHT

SEQ ID NO: 40 - protein, 4 aa, synthetic construct
  source 1..4: note = hinge
CPPC

SEQ ID NO: 41 - protein, 15 aa, synthetic construct
  source 1..15: note = hinge
CPEPKSCDTPPPCPR

SEQ ID NO: 42 - protein, 12 aa, synthetic construct
  source 1..12: note = hinge
ELKTPLGDTTHT

SEQ ID NO: 43 - protein, 10 aa, synthetic construct
  source 1..10: note = hinge
KSCDKTHTCP

SEQ ID NO: 44 - protein, 7 aa, synthetic construct
  source 1..7: note = hinge
KCCVDCP

SEQ ID NO: 45 - protein, 7 aa, synthetic construct
  source 1..7: note = hinge
KYGPPCP

SEQ ID NO: 46 - protein, 15 aa, Homo sapiens
  source 1..15: note = human IgG1 hinge
EPKSCDKTHTCPPCP

SEQ ID NO: 47 - protein, 12 aa, Homo sapiens
  source 1..12: note = human IgG2 hinge
ERKCCVECPPCP

SEQ ID NO: 48 - protein, 17 aa, Homo sapiens
  source 1..17: note = human IgG3 hinge
ELKTPLGDTTHTCPRCP

SEQ ID NO: 49 - protein, 12 aa, Homo sapiens
  source 1..12: note = human IgG4 hinge
SPNMVPHAHHAQ

SEQ ID NO: 50 - protein, 112 aa, synthetic construct
  source 1..112: note = CD3 zeta intracellular signaling domain amino acid sequence
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 336 bp, synthetic construct
  source 1..336: mol_type = other DNA; note = CD3 zeta intracellular signaling domain nucleic acid sequence
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 52 - protein, 112 aa, synthetic construct
  source 1..112: note = CD3 zeta (Q14K) intracellular signaling domain amino acid sequence
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 53 - DNA, 336 bp, synthetic construct
  source 1..336: mol_type = other DNA; note = CD3 zeta (Q14K) intracellular signaling domain nucleic acid sequence
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 54 - protein, 42 aa, synthetic construct
  source 1..42: note = 4-1BB costimulatory domain amino acid sequence
KRGRKKLLYIFKQPFMRPVQTTQEEDGCSCRFPEEEEGGCEL

SEQ ID NO: 55 - DNA, 126 bp, synthetic construct
  source 1..126: mol_type = other DNA; note = 4-1BB costimulatory domain nucleic acid sequence #1
aaacggggcagaaagaaactcctgtatatattcaaacaaccatttatgagaccagtacaaactactcaagaggaagacggctgtagctgccgatttccagaagaagaagaaggaggatgtgaactg

SEQ ID NO: 56 - DNA, 126 bp, synthetic construct
  source 1..126: mol_type = other DNA; note = 4-1BB costimulatory domain nucleic acid sequence #2
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 57 - protein, 41 aa, synthetic construct
  source 1..41: note = CD28 costimulatory domain amino acid sequence
RSKRSRLLHSDYMNMTPRRPGPTRKHYQPYAPPRDFAAYRS

SEQ ID NO: 58 - DNA, 123 bp, synthetic construct
  source 1..123: mol_type = other DNA; note = CD28 costimulatory domain nucleic acid sequence
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 59 - protein, 41 aa, synthetic construct
  source 1..41: note = CD28(YMFM) costimulatory domain amino acid sequence
RSKRSRLLHSDYMFMTPRRPGPTRKHYQPYAPPRDFAAYRS

SEQ ID NO: 60 - DNA, 123 bp, synthetic construct
  source 1..123: mol_type = other DNA; note = CD28(YMFM) costimulatory domain nucleic acid sequence
aggagtaagaggagcaggctcctgcacagtgactacatgttcatgactccccgccgccccgggcccacccgcaagcattaccagccctatgccccaccacgcgacttcgcagcctatcgctcc

SEQ ID NO: 61 - protein, 35 aa, synthetic construct
  source 1..35: note = ICOS costimulatory domain amino acid sequence
TKKKYSSSVHDPNGEYMFMRAVNTAKKSRLTDVTL

SEQ ID NO: 62 - DNA, 105 bp, synthetic construct
  source 1..105: mol_type = other DNA; note = ICOS costimulatory domain nucleic acid sequence #1
acaaaaaagaagtattcatccagtgtgcacgaccctaacggtgaatacatgttcatgagagcagtgaacacagccaaaaaatccagactcacagatgtgacccta

SEQ ID NO: 63 - DNA, 105 bp, synthetic construct
  source 1..105: mol_type = other DNA; note = ICOS costimulatory domain nucleic acid sequence #2
acaaaaaagaagtattcatccagtgtgcacgaccctaacggtgaatacatgttcatgagagcagtgaacacagccaaaaaatctagactcacagatgtgacccta

SEQ ID NO: 64 - protein, 35 aa, synthetic construct
  source 1..35: note = ICOS(YMNM) costimulatory domain amino acid sequence
TKKKYSSSVHDPNGEYMNMRAVNTAKKSRLTDVTL

SEQ ID NO: 65 - DNA, 105 bp, synthetic construct
  source 1..105: mol_type = other DNA; note = ICOS(YMNM) costimulatory domain nucleic acid sequence
acaaaaaagaagtattcatccagtgtgcacgaccctaacggtgaatacatgaacatgagagcagtgaacacagccaaaaaatccagactcacagatgtgacccta

SEQ ID NO: 66 - protein, 117 aa, synthetic construct
  source 1..117: note = CD2 costimulatory domain amino acid sequence
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 67 - DNA, 351 bp, synthetic construct
  source 1..351: mol_type = other DNA; note = CD2 costimulatory domain nucleic acid sequence
accaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaat

SEQ ID NO: 68 - protein, 48 aa, synthetic construct
  source 1..48: note = CD27 costimulatory domain amino acid sequence
QRRKYRSNKGESPVEPAEPCRYSCPREEEGSTIPIQEDYRKPEPACSP

SEQ ID NO: 69 - DNA, 144 bp, synthetic construct
  source 1..144: mol_type = other DNA; note = CD27 costimulatory domain nucleic acid sequence
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 70 - protein, 42 aa, synthetic construct
  source 1..42: note = OX40 costimulatory domain amino acid sequence
ALYLLRRDQRLPPDAHKPPGGGSFRTPIQEEQADAHSTLAKI

SEQ ID NO: 71 - DNA, 126 bp, synthetic construct
  source 1..126: mol_type = other DNA; note = OX40 costimulatory domain nucleic acid sequence
gccctgtacctgctccgcagggaccagaggctgccccccgatgcccacaagccccctgggggaggcagtttcaggacccccatccaagaggagcaggccgacgcccactccaccctggccaagatc

(19) United States
(12) Patent Application Publication
(10) Pub. No.: US 2024/0228959 A9
(43) Pub. Date: Jul. 11, 2024

(54) LARGE SCALE CAR-T IMMUNE CELL MANUFACTURING METHOD UTILIZING LENTIVIRAL VECTOR TRANSFECTION

(71) Applicant: Kite Pharma, Inc., Santa Monica, CA (US)

(72) Inventors: Felipe Bedoya, Eagleville, PA (US); David Barrett, Philadelphia, PA (US); Vijay Gopal Reddy Peddareddigari, Philadelphia, PA (US); Namrata Choudhari, Philadelphia, PA (US); Matthew VanPelt, Philadelphia, PA (US)

(73) Assignee: Kite Pharma, Inc., Santa Monica, CA (US)

(21) Appl. No.: 18/377,685

(22) Filed: Oct. 6, 2023

Related U.S. Application Data
(60) Provisional application No. 63/414,829, filed on Oct. 10, 2022.

(51) Int. Cl. C12N 5/0783; C12N 5/00; C12N 15/86

(52) U.S. Cl. CPC C12N 5/0636; C12N 5/0031; C12N 15/86; C12N 2527/00; C12N 2740/10043; C12N 2740/15043

(57) ABSTRACT

The present disclosure provides novel and efficient methods and lentiviral vectors for manufacturing a population of immune cells engineered to express a Chimeric Antigen Receptor (CAR), an engineered T cell receptor (TCR), and/or a nucleic acid sequence encoding a polypeptide that enhances the immune cell function, or a functional derivative thereof in less than 72 hours; engineered cells generated by the methods, compositions comprising said cells and methods of treating a disease or condition using said cells.

### CROSS-REFERENCE

The present application claims priority from U.S. Provisional Application No. 63/414,829, filed on Oct. 10, 2022, the contents of which are hereby incorporated by reference in their entirety for all purposes.

### SEQUENCE LISTING

The present application contains a Sequence Listing which is hereby incorporated by reference in its entirety. Said Sequence Listing XML, file was created on Jan. 9, 2024, is 82 bytes in size, and is named 125400_1745_Sequence_Listing.XML.

### FIELD

The present disclosure relates generally to efficient methods of manufacturing immune effector cells expressing a Chimeric Antigen Receptor (CAR), and/or engineered T cell receptor (TCR), and/or a nucleic acid sequence encoding a polypeptide that enhances the immune cell function, or a functional derivative thereof.

### BACKGROUND

Adoptive immunotherapy involves the transfer of autologous antigen-specific T-cells generated ex vivo back into a patient, and has been shown to be a promising strategy for the treatment of cancers, infections and auto-immune diseases. T-cells used for adoptive immunotherapy are primary cells engineered to express a Chimeric Antigen Receptor (CAR), or a recombinant T cell Receptor (TCR) and expand ex vivo to redirect primary immune cells against pathological cells, such as cancer cells. CARs are synthetic antibody-like molecules consisting of a targeting moiety that is associated with one or more signaling domains in a single fusion molecule, and are designed to convey antigen specificity to T cells. CARs have successfully allowed T cells to be redirected against antigens expressed at the surface of tumor cells from various malignancies, including lymphomas and solid tumors.

The manufacture of gene-modified T cells is currently a complex process. There exists a need for methods and processes for improving the production of the CAR- or TCR-expressing cell therapy products, enhancing product quality, and maximizing the therapeutic efficacy of CAR T cell immunotherapy. The present invention provides methods and compositions that address these needs.

### SUMMARY OF THE PRESENT TECHNOLOGY

One aspect of the present disclosure provides a method for manufacturing a population of engineered immune cells, the method comprising: (a) enriching a population of lymphocytes, a population immune cells or a population of CD4⁺ and CD8⁺ cells from blood obtained from a subject; (b) admixing the population of lymphocytes, the population of immune cells or the population of CD4⁺ and CD8⁺ cells with one or more buffer solutions; and (c) transfecting population of lymphocytes, the population of immune cells, or the population of CD4⁺ and CD8⁺ cells with an effective dose of a modifying agent; thereby generating a population of modified lymphocytes, a population of modified immune cells or a population of modified CD4⁺ and CD8⁺ cells. In some embodiments, steps 1(a)-(c) take place within 24 hours. In some embodiments, prior to the enriching of the population of immune cells or the population of CD4⁺ and CD8⁺ cells, the blood is separated into a plasma constituent, a mononuclear cell-containing layer, a platelet layer, and red blood cells by apheresis to produce an apheresis product selected from erythrocytapheresis, thrombapheresis, thrombocytapheresis, leukapheresis, stem cells, plasmapheresis, and plateletpheresis. In some embodiments, the population of immune cells or the population of CD4⁺ and CD8⁺ cells is enriched by apheresis, elutriation or gradient centrifugation.

Another aspect of the present disclosure provides a method for manufacturing a population of engineered immune cells, the method comprising: (a) enriching a population of lymphocytes, a population of immune cells or a population of CD4⁺ and CD8⁺ cells from a donor leukapheresis; (b) admixing the population of lymphocytes, the population of immune cells or the population of CD4⁺ and CD8⁺ cells with one or more buffer solutions; and (c) transfecting the population of lymphocytes, the population of immune cells or the population of CD4⁺ and CD8⁺ cells with an effective dose of a modifying agent, thereby generating a population of lymphocytes, a population of modified immune cells or a population of modified CD4⁺ and CD8⁺ cells. In some embodiments, steps 1(a)-(c) take place within 24 hours.

Another aspect of the present disclosure provides a method for manufacturing a population of engineered eukaryotic cells, the method comprising: (a) obtaining a population of eukaryotic donor cells from a subject; (b) admixing the population of eukaryotic donor cells with one or more buffer solutions; and (c) transfecting the population of eukaryotic donor cells with an effective dose of a modifying agent, thereby generating a population of modified eukaryotic donor cells. In some embodiments, steps 1(a)-(c) take place the same day.

In some embodiments of the method described herein, prior to the transfecting step (c), the population of immune cells, the population of CD4⁺ and CD8⁺ cells, or the population of eukaryotic donor cells is stimulated and/or activated with one or more stimulating agents.

In some embodiments of the method described herein, the modifying agent is selected from the group consisting of a small molecule agent, a biologic agent, a therapeutic, a protein, a peptide, a protein therapeutic, a peptide therapeutic, a chimeric antigen receptor, a heterologous T cell receptor, a viral vector, a vector, a retroviral vector, a lentiviral vector, an adenoviral vector, and an adeno-associated viral vector.

In some embodiments of the method described herein, the modifying agent is: (a) selected from a retroviral vector, a lentiviral vector, an adenoviral vector, or an adeno-associated viral vector; (b) a lentiviral vector; or (c) a retroviral vector.

In some embodiments of the method described herein, the population of immune cells, the population of CD4⁺ and CD8⁺ cells, or the population of eukaryotic donor cells is transfected with an effective dose of a lentiviral vector or a retroviral vector.

In some embodiments of the method described herein, the lentiviral vector or retroviral vector comprises a nucleic acid sequence encoding a chimeric antigen receptor (CAR), an engineered T cell receptor (TCR), and/or a nucleic acid sequence encoding a polypeptide that enhances the immune cell function, or a functional derivative thereof or produces a therapeutic protein.

In some embodiments of the method described herein, the population of immune cells or the population of eukaryotic donor cells is selected from the group consisting of mononuclear cells, Lymphocytes rich cells, B lymphocytes, T lymphocytes, CD4⁺ T lymphocytes, CD8⁺ T lymphocytes, dendritic cells, monocytes, natural killer (NK) cells, natural killer T (NKT) cells, T-regulatory cells, CD4⁺ T-helper cells, CD8⁺ cytotoxic T lymphocytes (CTLs), CD62L⁺ cells, CD27⁺ cells, CCR7⁺ cells, CD45RO⁻ cells, CD45RA⁺ cells, neutrophils, basophils, eosinophils, megakaryocytes, stem cells, hematopoietic stem cells (HSCs), hematopoietic progenitor cells (HPCs), CD34⁺ cells, CD34⁺ peripheral blood stem cells, lymphokine-activated killer cells (LAKs), tumor infiltrating lymphocytes (TILs), mesenchymal stem cells, mast cells, a monocyte, a macrophage, a neutrophil, a basophil, an eosinophil, a dendritic cell, a megakaryocyte, and combinations thereof.

In some embodiments of the method described herein, the concentration of the population of immune cells, the population of CD4⁺ and CD8⁺ cells, or the population eukaryotic donor cells is (a) at least about 0.7×10⁷, at least about 0.8×10⁷, at least about 0.9×10⁷ at least about 1×10⁷, at least about 2×10⁷, at least about 4×10⁷, at least about 6×10⁷, at least about 8×10⁷, at least about 1×10⁸, or at least about 5×10⁸ cells/mL; (b) from about 0.5×10⁶ cells/mL to about 4×10⁶ cells/mL; (c) from about 0.5×10⁶ cells/mL to about 1×10⁸ cells/mL; or (d) from about 4.0×10⁶ cells/mL to about 1×10⁸ cells/mL.

In some embodiments of the method described herein, transfecting is: (a) selected from the group consisting of viral transfection, transduction, non-viral transfection, and hybrid of viral and non-viral transfection; (b) selected from the group consisting of electroporation, laser beam, gene injection, sonoporation, magentofection, metal-coated nanoparticles, magnetic-conjugated adeno-associated virus, micro/nanoparticle-mediated transfection, lipofection, lipid-based transfection, anionic liposome, cationic liposome-mediated transfection, cationic polymer, polymer encapsulation, peptide mediated transfection, calcium phosphate, dendrimers, flowfection, photoporation, soluporation, transient cell-membrane disruption, deformation, squeezing, stretching, pinching, weakening, elongation, thinning, biolistic particle delivery systems and a combination thereof; (c) electroporation of a viral particle; (d) electroporation and viral transfection (transduction); (e) viral transfection and lipid-based transfection; or (f) viral transfection and liposome based transfection.

In some embodiments of the method described herein, (a) the population of modified immune cells, the population of modified CD4⁺ and CD8⁺ cells, or the population of modified eukaryotic donor cells is not activated with one or more stimulating agents following or before transfection; and (b) the population of modified immune cells, the population of modified CD4⁺ and CD8⁺ cells, or the population of modified eukaryotic donor cells is not expanded ex vivo following transfection.

In some embodiments, the method described herein further comprises stimulating and activating the population of modified immune cells, the population of modified CD4⁺ and CD8⁺ cells, or the population of modified eukaryotic donor cells with one or more stimulating agents to produce a population of activated modified immune cells, a population of activated modified CD4⁺ and CD8⁺ cells, or a population of activated modified eukaryotic cells.

In some embodiments, the method described herein further comprises expanding the population of activated lymphocytes, the population of activated modified immune cells, the population of activated modified mononuclear cells, the population of activated modified CD4⁺ and CD8⁺ cells, or the population of activated modified eukaryotic donor cells for a predetermined time to produce a population of engineered lymphocytes, a population of engineered immune cells, a population of engineered CD4⁺ and CD8⁺ cells, or a population of engineered eukaryotic donor cells. In some embodiments, the expanding step is performed: (a) under shaking conditions or rotating conditions; (b) in a closed system; (c) using a serum-free culture medium; and/or (d) in the presence of one or more stimulating agents.

In some embodiments, the population of activated modified immune cells, the population of activated modified CD4⁺ and CD8⁺ cells, or the population of activated modified eukaryotic donor cells are expanded for at least about 2-fold, at least about 3-fold, at least about 4-fold, at least about 5-fold, at least about 6-fold, at least about 7-fold, at least about 8 fold, at least about 10-fold, at least about 15-fold, at least about 20-fold, or at least about 25-fold.

In some embodiments, the method described herein further comprises harvesting the population of modified lymphocytes, the population of modified immune cells, the population of modified CD4⁺ and CD8⁺ cells, or the population of modified eukaryotic donor cells for cryopreservation or administration. In some embodiments, harvesting comprises selecting and enriching for the engineered lymphocytes, engineered immune cells, engineered CD4⁺ and CD8⁺ cells, or engineered donor eukaryotic cells. In some embodiments, harvesting further comprises formulating the engineered lymphocytes, the engineered immune cells, the engineered CD4⁺ and CD8⁺ cells, or the engineered donor eukaryotic cells for cryopreservation or administration to a subject in need thereof.

In some embodiments, the predetermined time for expanding the population of modified activated cells described herein (lymphocytes, immune cells, t mononuclear cells, CD4⁺ and CD8⁺ cells, or eukaryotic donor cells) is: (a) less than about 24 hours; less than about 30 hours; less than about 48 hours; less than about 72 hours; less than about 96 hours; or less than about 120 hours; (b) less than about, 0.5 hour, less than about 1 hour, less than about 2 hours, less than about 3 hours, less than about 4 hours, less than about 5 hours, less than about 6 hours, less than about 7 hours, less than about 8 hours, less than about 9 hours, less than about 10 hours, less than about 11 hours, less than about 12 hours, less than about 13 hours, less than about 14 hours, less than about 15 hours, less than about 16 hours, less than about 17 hours, less than about 18 hours, less than about 19 hours, less than about 20 hours, less than about 21 hours, less than about 22 hours, or less than about 23 hours; or (c) about 1 day, about 2 days, about 3 days, about 4 days, about 5 days, about 6 days, about 7 days, about 8 days, about 9 days, about 10 days, about 11 days, about 12 days, about 13 days, about 14 days, or more days.

In some embodiments of the method described herein, the time from enriching and/or obtaining the population of lymphocytes, the population of immune cells, the population of CD4⁺ and CD8⁺ cells, or the population of eukaryotic donor cells to harvesting the engineered immune cells, the engineered CD4⁺ and CD8⁺ cells, or the engineered eukaryotic donor cells is: (a) about 72 hours or less; (b) from about 18 hours to about 72 hours, from about 18 hours to about 36 hours, from about 18 hours to about 24 hours, from about 24 hours to about 72 hours, from about 24 hours to about 36 hours, or from about 36 hours to about 72 hours; (c) less than about 2 hours, less than about 3 hours, less than about 4 hours, less than about 5 hours, less than about 6 hours, less than about 7 hours, less than about 8 hours, less than about 9 hours, less than about 10 hours, less than about 11 hours, less than about 12 hours, less than about 13 hours, less than about 14 hours, less than about 15 hours, less than about 16 hours, less than about 17 hours, less than about 18 hours, less than about 19 hours, less than about 20 hours, less than about 21 hours, less than about 22 hours, or less than about 23 hours; (d) about 1 day, about 2 days, about 3 days, about 4 days, about 5 days, about 6 days, about 7 days, about 8 days, about 9 days, about 10 days, about 11 days, about 12 days, about 13 days, about 14 days, or more days; or (e) about 1 day, about 3 days, about 4 days, about 5 days, or about 6 days.

In some embodiments of the method described herein, the electroporating step, the activating step and/or the expanding step are performed in a closed system, a semi-closed, and/or a functionally closed system. In some embodiments, the closed system is selected from the group consisting of a closed bag system, an automated closed cell sample processing system, and a bioreactor. In some embodiments, (a) the one or more stimulating agents are selected from the group consisting of agonistic antibodies, cytokines, recombinant costimulatory molecules, anti-CD3 antibodies or fragments thereof, anti-CD28 antibodies or fragments, small drug inhibitors, and/or a combination thereof; (b) the one or more stimulating agents are cytokines selected from the group consisting of Interleukin-2 (IL-2), Interleukin-3 (IL-3), Interleukin-6 (IL-6), Interleukin-7 (IL-7), Interleukin-7 receptor (IL-7R), Interleukin-11 (IL-11), Interleukin-12 (IL-12), Interleukin-15 (IL-15), Interleukin-15 receptor (IL-15R), Interleukin-18 (IL-18), Interleukin-18 receptor (IL-18R), Interleukin-21 (IL-21), granulocyte macrophage colony stimulating factor, alpha, beta or gamma interferon, erythropoietin, and a combination thereof. In some embodiments, the one or more stimulating agents are conjugated to a bead or a nanostructure.

In some embodiments of the method described herein, (a) the one or more stimulating agents are anti-CD3 and anti-CD28 antibodies or fragments thereof; (b) the one or more stimulating agents are anti-CD3 and anti-CD28 antibodies or fragments thereof and one or more cytokines; (b) the nanostructure is a nanomatrix; (c) the cytokine is selected from IL-2, IL-7, IL-6, IL-15, IL-15Ra, or IL-21; (d) the cytokine is selected from IL-15 and IL-7; IL-7 and IL-21; IL-7 and IL-2; IL-15 and IL-2; IL-7, IL-15, and IL-21; IL-15 and IL-15Ra; or IL-7, IL-15 and IL-15Ra; and/or (e) the one or more stimulating agents are a nanomatrix and one or more cytokines. In some embodiments, the nanomatrix (a) comprises a matrix of mobile polymer chains, and anti-CD3 and anti-CD28 antibodies or fragments thereof; or (c) is 1 to 500 nm in size.

In some embodiments of the method described herein, the effective dose of the retroviral vector or lentiviral vector comprises a multiplicity of infection (MOI) of about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.25, about 1.5, about 2.0, about 3.0, about 4.0, or about 5.0.

In some embodiments, the effective dose of the retroviral vector or lentiviral vector comprises: (a) about 2 ul of the lentiviral vector at a MOI of about 0.08; (b) about 5 ul of the lentiviral vector at a MOI of about 0.2; or (c) about 10 ul of the lentiviral vector at a MOI of about 0.4.

In some embodiments of the method described herein, the lentiviral vector is based on a virus selected from the group consisting of a retrovirus, an alpharetrovirus, a betaretrovirus, a gammaretrovirus, a deltaretrovirus, and an epsilonretrovirus. In some embodiments, the lentiviral vector is based on a Human immunodeficiency virus (HIV), an Equine infectious anaemia virus (EIAV), a visna-maedi virus (VMV) virus, a caprine arthritis-encephalitis virus (CAEV), a feline immunodeficiency virus (Hy), a bovine immune deficiency virus (BIV), a VISNA virus, and a simian immunodeficiency virus (SIV).

In some embodiments, the lentiviral vector is pseudotyped with an envelope glycoprotein (Env) from a virus selected from the group consisting of a murine leukemia virus (MLV), a vesicular stomatitis virus (VSV) Indiana strain, VSV New Jersey strain, Cocal virus, Chandipura virus, Piry virus, spring viremia of carp virus (SVCV), Sigma virus, infectious hematopoietic necrosis virus (IHNV), Mokola virus, rabies virus CVS virus, Isfahan virus, Alagoas virus, Calchaqui virus, Jurona vrus, La Joya virus, Maraba virus, Feline Endogenous Retrovirus (RD114) Envelope Protein, Perinet virus, Yug Bugdanovac virus, a prototypic foamy virus (PFV), and gibbon ape leukemia virus (GaLV).

In some embodiments, the lentiviral vector is pseudotyped with an envelope glycoprotein (Env) selected from the group consisting of vesicular stomatitis virus (VSV) Indiana strain, VSV New Jersey strain, and Cocal virus.

In some embodiments, the lentiviral vector comprises a heterologous viral envelope protein (Env) selected from the group consisting of a VSV-G of the Indiana strain, VSV-G of the New Jersey strain, the Cocal virus envelope protein, the Isfahan virus envelope protein, Chandipura virus envelope protein, Pyri virus envelope protein, a murine leukemia virus (MLV) envelope glycoprotein, a SVCV virus envelope protein, and a variant thereof. In some embodiments, the lentiviral vector comprises a nucleotide sequence encoding the VSV-G envelope protein, or a VSV G protein variant. In some embodiments of the method described herein, the lentiviral vector is a lentiviral particle.

In some embodiments of the method described herein, the CAR comprises an antigen-binding domain, a transmembrane domain, a co-stimulatory domain, and an intracellular domain, and wherein the antigen-binding domain is selected from the group consisting of (a) a full-length antibody or antigen-binding fragment thereof, (b) a Fab, (c) a single-chain variable fragment (scFv), and (d) a single-domain antibody.

In some embodiments, the antigen-binding domain specifically binds a target antigen selected from the group consisting of CD4, CD5, CD19, CD20, CD22, CD79b, CD79a, CD33, CD30, CD70, BCMA, GPC2, CD123, CD133, EGFR, EGFRvIII, mesothelin, HER2, PSMA, PSCA, FAP, CEA, GD2, IL-13Ra2, glypican-3, CIAX, LI-CAM, CA 125, CTAG1B, TnMUC1, Mucin 1, and Folate receptor-alpha (FRa), GFR Alpha-4, NYESO, WT1, (AFP)/HLA-A2, AXL, B7-H3, CA-IX, CD3, CD7, CD8, CD38, CD44v6, CD80, CD86, CD117, CD147, CD276, CEA, claudin 18.2, c-Met, DLL3, DR5, EpCAM, EphA2, FAP, folate binding protein (FBP), Glycolipid F77, glypican-3 (GPC3), glypican-2, HLA-A2, ICAMI, IL3Ra, LAGE-I, Lewis Y, LMPI (EBV), MAGE-A1, MAGE-A3, MAGE-A4, Melan A, MG7 (glycosylated CEA), MMP, MUCI, Nectin4/FAP, NKG2D-Ligands, MIC-A, MIC-B, ULBPs I to 6, NY-ESO-1, P16, PD-L1, ROR1, ROR2, TIM-3, TM4SF1, VEGFR2, and any combination thereof.

In some embodiments, the CAR transmembrane domain is selected from the group consisting of an artificial hydrophobic sequence, a transmembrane domain of a type I transmembrane protein, an alpha, beta, or zeta chain of a T cell receptor, CD28, CD3 epsilon, CD45, CD4, CD5, CD8, CD9, CD16, CD22, CD33, CD37, CD64, CD80, CD86, OX40 (CD134), 4-1BB (CD137), ICOS (CD278), or CD154, and a transmembrane domain derived from a killer immunoglobulin-like receptor (KIR).

In some embodiments, the costimulatory domain is an intracellular domain of a protein selected from the group consisting of a TNFR superfamily protein, CD27, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and killer immunoglobulin-like receptor (KIR).

In some embodiments, the intracellular signaling domain comprises an intracellular domain selected from the group consisting of cytoplasmic signaling domains of a human CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d. In some embodiments of the method described herein, the CAR further comprises a hinge region.

Another aspect of the present disclosure provides a method for delivering a nucleic acid encoding a chimeric antigen receptor (CAR), an engineered T Cell receptor, or a therapeutic protein to a cell, the method comprising introducing into the cell a transfer plasmid comprising: (a) a polynucleotide sequence encoding at least one heterologous viral envelope protein engineered by the method described herein; (b) a polynucleotide sequence encoding at least one retroviral rev protein; (c) a polynucleotide sequence encoding at least one retroviral gag protein and a retroviral pol protein; and/or (d) a polynucleotide sequence encoding the chimeric antigen receptor, the engineered T cell receptor (TCR), or the therapeutic protein. In some embodiments, at least part of one or more regions of the retroviral genome essential for replication is mutated.

Another aspect of the present disclosure provides a lentiviral vector particle generated by the method described herein.

Another aspect of the present disclosure provides a method of introducing a modification to a cell, the method comprising electroporating a cell with an effective dose of the lentiviral vector particle described herein or made by the methods described herein, thereby generating a modified cell. In some embodiments, the cell is contacted with the effective dose of the lentiviral vector prior to electroporation. In some embodiments, the cell is contacted with the effective dose of the lentiviral vector for up to about 4 hours after electroporation. In some embodiments, the cell is contacted with the effective dose of the lentiviral vector for: (a) at least about 5-30 minutes, at least about 25-50 minutes; at least about 5-60 minutes, at least about 5-12 minutes, at least about 60-120 minutes, at least about 120-240 minutes after electroporation; (b) at least about 1 minute, at least about 2 minutes, at least about 5 minutes, at least about 10 minutes, at least about 20 minutes, at least about 30 minutes, at least about 45 minutes, at least about 60 minutes, at least about 75 minutes, at least about 90 minutes, at least about 100 minutes, at least about 110 minutes, at least about 120 minutes, at least about 150 minutes, at least about 160 minutes, at least about 170 minutes, at least about 180 minutes, at least about 190 minutes, at least about 200 minutes, at least about 220 minutes, or at least about 240 minutes after electroporation.

In some embodiments, the cell is selected from the group consisting of immune cells, eukaryotic donor cells, mononuclear cells, enriched lymphocytes, B lymphocytes, T lymphocytes, CD4⁺ T lymphocytes, CD8⁺ T lymphocytes, dendritic cells, monocytes, natural killer (NK) cells, natural killer T (NKT) cells, T-regulatory cells, CD4⁺ T-helper cells, CD8⁺ cytotoxic T lymphocytes (CTLs), CD62L⁺ cells, CD27⁺ cells, CCR7⁺ cells, CD45RO⁻ cells, CD45RA⁺ cells, neutrophils, basophils, eosinophils, megakaryocytes, stem cells, hematopoietic stem cells (HSCs), hematopoietic progenitor cells (HPCs), CD34⁺ cells, CD34⁺ peripheral blood stem cells, lymphokine-activated killer cells (LAKs), tumor infiltrating lymphocytes (TILs), circulating tumor specific T cells, mesenchymal stem cells, mast cells, a monocyte, a macrophage, a neutrophil, a basophil, an eosinophil, a dendritic cell, a megakaryocyte, and combinations thereof.

In some embodiments, the cell is: (a) a lymphoid cell selected from the group consisting of a T cell, a B cell, a natural killer (NK) cell, a CD8⁺ T cell, a CD4⁺ T cell, a cytotoxic T lymphocyte, a regulatory T cell, and any combination thereof; (b) a myeloid cell selected from the group consisting of a monocyte, a macrophage, a neutrophil, a basophil, an eosinophil, a dendritic cell, a megakaryocyte, and any combination thereof; (c) a stem cell, an hematopoietic stem cell, an hematopoietic progenitor cell, a CD34⁺ cell, or CD34⁺ peripheral blood stem cell.

In some embodiments, the effective dose of the lentiviral vector particle comprises about 0.5 ul, about 1 ul, about 1.5 ul, about 2 ul, about 2.5 ul, about 3 ul, about 3.5 ul, about 4 ul, about 5 ul, about 6 ul, about 7 ul, about 8 ul, about 9 ul, about 10 ul, about 15 ul, or about 20 ul of the lentiviral vector. In some embodiments, the effective dose of the lentiviral vector particle comprises a multiplicity of infection (MOI) of about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.25, about 1.5, about 2.0, about 3.0, about 4.0, or about 5.0.

In some embodiments, the effective dose of the lentiviral vector particle comprises: (a) about 2 ul of lentiviral vector particle at a MOI of about 0.08; (b) about 5 ul of lentiviral vector particle at a MOI of about 0.2; or (c) about 10 ul of lentiviral vector particle at a MOI of about 0.4.

One aspect of the present disclosure provides a modified cell, a modified immune cell, a modified CD4⁺ and CD8⁺ cell, or a modified eukaryotic donor cell engineered by the methods described herein.

One aspect of the present disclosure provides a population of modified cells, a population of modified immune cells, a population of modified CD4⁺ and CD8⁺ cells, or a population modified eukaryotic donor cells engineered by the methods described herein.

One aspect of the present disclosure provides a modified cell, a modified immune cell, a modified CD4⁺ and CD8⁺ cell, or a modified eukaryotic donor cell comprising the lentiviral vector described herein.

One aspect of the present disclosure provides a population of modified cells, a population of modified immune cells, a population of modified CD4⁺ and CD8⁺ cells or a population modified eukaryotic donor cells comprising a lentiviral vector described herein.

In some embodiments, the modified CD4⁺ and CD8⁺ cell, or the modified eukaryotic donor cell engineered described herein or generated by the methods described herein for use in the production of a protein of interest. In some embodiments, the protein of interest is selected from the group consisting of an industrial protein, or a therapeutic protein. In some embodiments, the protein of interest is selected from the group consisting of enzymes, regulatory proteins, receptors, peptides, peptide hormones, cytokines, membrane or transport proteins, vaccine antigens, antigen-binding proteins, immune stimulatory proteins, allergens, full-length antibodies or antibody fragments or derivatives; single chain antibodies, (scFv), Fab fragments, Fv fragments, single domain antibodies (VH or VL fragment), domain antibodies, camelid single domain antibodies (VHH), nanobodies and a combination thereof.

One aspect of the present disclosure provides a composition comprising: (a) a modified cell, a modified immune cell, a modified CD4⁺ and CD8⁺ cell, or a modified eukaryotic donor cell described herein or engineered by the methods described herein; (b) a population of modified cells, a population of modified immune cells, a population of modified CD4⁺ and CD8⁺ cells, or a population modified eukaryotic donor cells described herein or engineered by the methods described herein; or (c) a lentiviral vector described herein. In some embodiments, the composition further comprises a pharmaceutically acceptable excipient.

One aspect of the present disclosure provides a method of treating a disease or condition in a subject, the method comprising administering to the subject in need thereof a therapeutically effective amount of: (a) the modified cell, the modified immune cell, the modified CD4⁺ and CD8⁺ cell, or the modified eukaryotic donor cell described herein or engineered by the methods described herein; (b) the population of modified cells, the population of modified immune cells, the population of modified CD4⁺ and CD8⁺ cells, or the population of modified eukaryotic donor cells described herein or engineered by the methods described herein; or (c) the composition described herein, thereby treating the disease or condition in the subject.

In some embodiments, the disease or condition is selected from the group consisting of viral infection, a bacterial infection, a parasitic infections, a cancer, a malignancy, a non-cancerous condition, an autoimmune disease, a fibrotic disease, Alzheimer's disease, protein deficiency conditions, and factor VIII deficiency.

In some embodiments, the cancer is selected from the group consisting of breast cancer, triple-negative breast cancer, prostate cancer, ovarian cancer, glioma, glioblastoma, renal cell carcinoma, kidney cancer, mesothelioma, cervical cancer, skin cancer, pancreatic cancer, colorectal cancer, renal cancer, liver cancer, lung cancer, lung adenocarcinoma, gallbladder cancer, colon cancer, cervical squamous cell carcinoma, non-small cell lung cancer, small cell lung cancer, Merkel cell carcinoma, hepatocellular carcinoma, esophagus cancer, brain cancer, melanoma, Hodgkin's lymphoma, non-Hodgkin's lymphoma, urothelial carcinoma, gastric cancer, blood cancers, lymphoma, leukemia, multiple myeloma, diffuse large B-cell lymphoma, follicular lymphoma, mantle cell lymphoma, acute myelogenous leukemia, B-cell acute lymphoblastic leukemia (ALL), pre-B ALL, and any combination thereof. In some embodiments, the modified immune cell, the modified CD4⁺ and CD8⁺ cell, or the modified eukaryotic donor cell is: (a) autologous to the subject; (b) allogeneic to the subject; or (c) a xenogeneic to the subject. In some embodiments, the modified cell, the modified immune cell, the modified CD4⁺ and CD8⁺ cell, or the modified eukaryotic donor cell is allogeneic to the subject. In some embodiments, the subject is a human.

One aspect of the present disclosure provides a method of producing a therapeutic protein, the method comprising: (a) manufacturing a population of engineered immune cells, or a population of engineered eukaryotic cells comprising the therapeutic protein using the methods described herein; (b) harvesting the therapeutic protein; and (c) isolating and purifying the therapeutic protein.

In some embodiments, the therapeutic protein is selected from the group consisting of enzymes, regulatory proteins, receptors, peptides, peptide hormones, cytokines, membrane or transport proteins, vaccine antigens, antigen-binding proteins, immune stimulatory proteins, allergens, full-length antibodies or antibody fragments or derivatives; single chain antibodies, (scFv), Fab fragments, Fv fragments, single domain antibodies (VH or VL fragment), domain antibodies, camelid single domain antibodies (VHH), nanobodies, and a combination thereof.

One aspect of the present disclosure provides a kit comprising: (a) a population of modified immune cells or a population of modified CD4⁺ and CD8⁺ cells, or a population of engineered by the methods described herein; or (b) a lentiviral vector described herein.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1 shows a schematic illustrating a rapid T-cell engineering platform (1A) using an integrating lentiviral vector (LVV) transfection at D0. This platform 1A comprises obtaining and processing a donor leukapheresis, selecting for a CD4⁺ and CD8⁺ cell, lentiviral vector transfection in a closed system on Day 0, which is followed by cell activation and ex vivo culture and expansion from at least about 1 hour to about 72 hours prior to harvest (e.g., culture and expansion from D0-D3). Harvested engineered T cells are either cryopreserved or administered to a subject in need thereof.

FIG. 2 shows a schematic illustrating a rapid T-cell engineering platform (1B) using the integrating lentiviral vector (LVV) transfection at D0 of FIG. 1, where the process starts with a donor whole blood rather than a donor leukapheresis.

FIG. 3 shows a schematic illustrating a rapid immune cell engineering platform (1C) using an integrating lentiviral vector (LVV) transfection at D0. This platform 1C comprises obtaining and processing a donor leukapheresis, selecting for immune cells (white blood cells and/or other immunes), lentiviral vector transfection in a closed system on Day 0, which is followed by cell activation and ex vivo culture and expansion from at least about 1 hour to about 72 hours prior to harvest (e.g., culture and expansion from D0-D3). Harvested engineered immune cells are either cryopreserved or administered to a subject in need thereof.

FIG. 4 shows a schematic illustrating a rapid immune cell engineering platform (1D) using the integrating lentiviral vector (LVV) transfection at D0 of FIG. 3, where the process starts with a donor whole blood rather than a donor leukapheresis.

FIG. 5 shows a schematic illustrating a rapid no-ex vivo culture T-cell engineering platform (2A) using an integrating lentiviral vector (LVV) transfection at D0. This platform 2A comprises obtaining and processing a donor leukapheresis, selecting for CD4⁺ and CD8⁺, lentiviral vector transfection in a closed system on Day 0, which is followed by cell harvest with no culture or expansion following transfection. Harvested engineered T cells are either cryopreserved or administered to a subject in need thereof.

FIG. 6 shows a schematic illustrating a rapid no-ex vivo culture T-cell engineering platform (2B) using the integrating lentiviral vector (LVV) transfection at D0 of FIG. 5, where the process starts with a donor whole blood rather than a donor leukapheresis.

FIG. 7 shows a schematic illustrating a rapid no-ex vivo culture immune cell engineering platform (3A) using an integrating lentiviral vector (LVV) transfection at D0. This platform 3A comprises obtaining and processing a donor leukapheresis, selecting for immune cells (white blood cells and/or other immunes), lentiviral vector transfection in a closed system on Day 0, which is followed by cell harvest. Harvested engineered cells are either cryopreserved or administered to a subject in need thereof.

FIG. 8 shows a schematic illustrating a rapid no-ex vivo culture immune cell engineering platform (3B) using an integrating lentiviral vector (LVV) transfection at D0 of FIG. 5, where the process starts with a donor whole blood rather than a donor leukapheresis.

FIG. 9 shows a schematic illustrating a rapid eukaryotic cell engineering platform (4A) using an integrating lentiviral vector (LVV) transfection at D0. This platform 4A comprises obtaining and processing donor eukaryotic cells (e.g., mammalian cells, human cells), selecting for a particular cell type (epithelial, mesenchymal, fibroblast, neuronal cells, or stem cells), and lentiviral vector transfection in a closed system on Day 0, which is followed by cell activation and ex vivo culture and expansion from at least about 1 hour to about 72 hours prior to harvest (e.g., culture and expansion from D0-D3). Harvested engineered eukoaryotic cells are either cryopreserved or used immediately.

FIG. 10 shows a schematic illustrating a rapid no-ex vivo culture eukaryotic cell engineering platform (4B) using an integrating lentiviral vector (LVV) transfection at D0. This platform 4B comprises obtaining and processing donor eukaryotic cells, selecting for a particular cell type (epithelial, mesenchymal, fibroblast, neuronal cells, or stem cells), and lentiviral vector transfection in a closed system on Day 0, which is followed by cell harvest. Harvested eukaryotic cells are either cryopreserved or used immediately.

FIG. 11 shows a bar graph summarizing results of CD19 CART cells-Nalm6 Co-culture Assay illustrating that CD19 CAR T cells produced by the novel manufacturing process disclosed herein (Electric CAR T cells), that uses transfection of lentivirus, were highly cytotoxic to target tumor cells as illustrated by Nalm6 killing in vitro. Representative mean values of two-three independent experiments (n=2-3) are shown. Mean±SEM ***p<0.005.

### DETAILED DESCRIPTION

### I. Overview

A. Canonical Manufacturing Process

Adoptive cell transfer therapy with T cells, especially with T cells transduced with Chimeric Antigen Receptors (CARs), has shown promise in several hematologic cancer trials. Despite this success, the manufacture of gene-modified T cells remains a complex, costly, and long process. A canonical CAR T cell manufacturing process starts with the enrichment of T cells from a fresh or a cryopreserved leukapheresis sample. The enrichment usually comprises using positive or negative selection. Enriched T cells are then activated using anti-CD3/anti-CD28 antibody coated beads (e.g., Dynabeads®), anti-CD3/anti-CD28 antibody coated polymers, nanoparticles, nanocolloids and/or a co-activator selected from the group consisting of a reagent that stimulates ICOS, CD27, HVEM, LIGHT, CD40, 4-1BB, 0X40, DR3, GITR, CD30, TIM1, CD2, or CD226. Once activated, T cells are transfected with a nucleic acid molecule encoding a CAR molecule or an exogenous TCR either immediately or up to 18 hours after the initiation of the activation step. Generally, T cells are transduced with a lentiviral vector comprising a nucleic acid molecule encoding the CAR molecule or the exogenous TCR. In some cases, the cells are electroporated with an in vitro transcribed RNA. Transfected cells are then cultured (i.e., expanded) in vitro for 1, 2, 3, 4, 5, 6, 7, 8, 9, 10, 11, 12 days, or more. Once culture has proceeded for the predetermined number of days, the cells are harvested. Harvesting cells includes mechanically resuspending engineered cells (e.g., T cells) by swirling or pipetting or otherwise agitating; and removing simulating/activating reagents with appropriate buffers. The cells are also washed to remove unnecessary reagents and reformulated in cryopreservation media or for immediate administration to a subject. The cells are cryopreserved until needed for administration.

The invention of the present disclosure is directed to a significant improvement in this canonical manufacturing process, where one or more manufacturing steps are deleted or shortened. It was surprisingly discovered that this shortened, more efficient immune effector cell manufacturing process resulted in highly desirable and effective compositions. The shortened process relies on lentiviral transfection rather than the conventional lentiviral transduction.

B. Improved 1 Day Manufacturing Process Using Lentiviral Transfection.

Provided herein are novel efficient methods of manufacturing immune effector cells (for example, T cells or NK cells) engineered to express a CAR, or TCR, methods for treating a disease (e.g., cancer) in a subject using the engineered cells, and lentiviral vectors for use in the methods described herein. In some embodiments, the manufacturing process disclosed herein may manufacture immune effector cells engineered to express a CAR or a TCR in less than about 24 hours (e.g., less than about 20 hours, less than about 15 hours, less than about 10 hours, less than about 5 hours, or less than about 3 hours, or any other time frame less than about 24 hours described herein). In some embodiments, the manufacturing process disclosed herein may manufacture immune effector cells engineered to express a CAR or a TCR in less than about 72 hours (e.g., less than about 24 hours, less than about 30 hours, less than about 40 hours, less than about 48 hours, less than about 60 hours, or less than about 72 hours, or any other time frame less than about 24 hours described herein).

One aspect of the present disclosure provides a method for manufacturing a population of engineered immune cells, the method comprising: (1) enriching a population of lymphocytes, a population immune cells or a population of CD4⁺ and CD8⁺ cells from donor whole blood or donor leukapheresis (e.g., frozen or fresh); (2) admixing the population of lymphocytes, the population of immune cells or the population of CD4⁺ and CD8⁺ cells with one or more buffer solutions; and (3) transfecting population of lymphocytes, the population of immune cells, or the population of CD4⁺ and CD8⁺ cells with an effective dose of a modifying agent; thereby generating a population of modified lymphocytes, a population of modified immune cells or a population of modified CD4⁺ and CD8⁺ cells. In some embodiments, steps 1(a)-(c) take place within about 24 hours or less.

Another aspect of the present disclosure provides a method for manufacturing a population of engineered eukaryotic cells, the method comprising: (1) obtaining a population of eukaryotic donor cells; (2) admixing the population of eukaryotic donor cells with one or more buffer solutions; and (3) transfecting the population of eukaryotic donor cells with an effective dose of a modifying agent, thereby generating a population of modified eukaryotic donor cells.

In some embodiments, the population of modified lymphocytes, the population of modified immune cells, the population of modified CD4⁺ and CD8⁺ cells, or the population of modified eukaryotic donor cells is not activated with one or more stimulating agents or expanded following transfection.

In some embodiments, the methods described herein further comprise stimulating and/or activating with one or more stimulating agents, and expanding the population of modified lymphocytes, the population of modified immune cells, the population of modified CD4⁺ and CD8⁺ cells, or the population of modified eukaryotic donor cells to produce a population of activated modified immune cells, a population of activated modified CD4⁺ and CD8⁺ cells, or a population of activated modified eukaryotic cells. In one aspect, the methods described herein further comprise stimulating and/or activating the population of lymphocytes, the population of immune cells, the population of CD4⁺ and CD8⁺ cells, or the population of eukaryotic donor cells with one or more stimulating agents prior to the transfecting step.

The immunotherapy (e.g., adoptive cell transfer therapy) methods of treating a disease or a disorder described herein employ a well-established and powerful system (apheresis and products thereof) to provide an acute, reliable, and efficient production of clinical-grade CAR- or TCR-engineered cells that can be immediately administered to a subject on site. The immunotherapy using the Electric CAR T cells generated by the methods disclosed herein can reduce the entire adoptive cell transfer process to about a single day (e.g., about 24 hours), or about 3 days or less (e.g., about 72 hours or less). As such, the manufacturing time is shortened from about 12-15 days to about 1 day, about 2 days or less, or about 3 days or less. The manufacturing processes disclosed herein are efficient because fewer cells are required to generate CAR T cells. For example, traditional manufacturing processes require up to about 300 million cells, while the manufacturing processes described herein work efficiently with about 3 million cells—a 100× decrease in cell number. This is because the cells from the present method are very fresh and uncompromised, and therefore more potent (e.g., less ex vivo/in vitro handling).

The manufacturing processes disclosed herein improve the production of the CAR- or TCR-expressing cell therapy product, enhance CAR T cells product quality, and maximize the therapeutic efficacy of the CAR T cells product in the following ways.

First, the manufacturing processes disclosed herein reduce the turnaround manufacturing time to about 1 day (e.g., 24 hours) or less (or about 2 days or less, or about 3 days or less) as compared to canonical manufacturing processes (e.g., about 12 days). This short turnaround time allows for a timely infusion of the CAR T cells (e.g., CD19, mesothelin, PSMA, TnMUC, BCMA, or GPC2 CAR-T cell) to patients. Moreover, the manufacturing processes preserve putative stem memory T (Tstem) cells, a cellular subset associated with improved antitumor efficacy. The majority of unstimulated CAR T cells generated by the manufacturing processes disclosed herein maintained a less differentiated phenotype (e.g., over 50% of transfected CAR T cells were naïve CAR T cells (CD45RO⁻ CCR7⁺) when compared to less than 10% of the CAR T cells population in stimulated CAR T cells. The high population of naïve Electric CAR T cells is a desirable improvement because these CAR T cells conserved a non-activated (i.e., less differentiated) phenotype, which is known to favor CART cell persistence and potency in cancer patients.

In some embodiments, the CART cells manufactured by the methods disclosed herein may be administered to a subject with minimal ex vivo expansion, for example, less than about 1 day, less than about 12 hours, less than about 8 hours, less than about 6 hours, less than about 4 hours, less than about 3 hours, less than about 2 hours, less than about 1 hour, or no ex vivo expansion. If desired, in other aspects the manufacturing can be less than about 5 days, less than about 4 days, less than about 3 days, or less than about 2 days. Accordingly, the methods described herein provide a rapid manufacturing process of making improved CAR-expressing cell products for use in treating a disease in a subject.

C. Summary of Experimental Results

The shortened manufacturing processes disclosed herein were made possible by a new strategy of transducing immune cells with a lentiviral vector comprising a nucleic acid encoding a CAR, a TCR and/or a polypeptide that enhances the immune cell function, or a functional derivative thereof.

First, in one embodiment, the manufacturing processes rely on a hybrid transfection method that combines a biological transfection (virus based transfection or viral transducing) and a physical transfection (electroporation). Specifically, lentiviral particles are electroporated into immune cells. Second, the lentiviral vectors used to transduce the cells are replication incompetent. The electroporation of lentiviral particles into cells speed up the viral transfection/transduction, which was followed by a standard cell culture process or an ultra-fast process with no cell culture. Electroporating the lentiviral vector into the cells also significantly reduced the amount of lentiviral vector used for optimal transfection using the process disclosed herein.

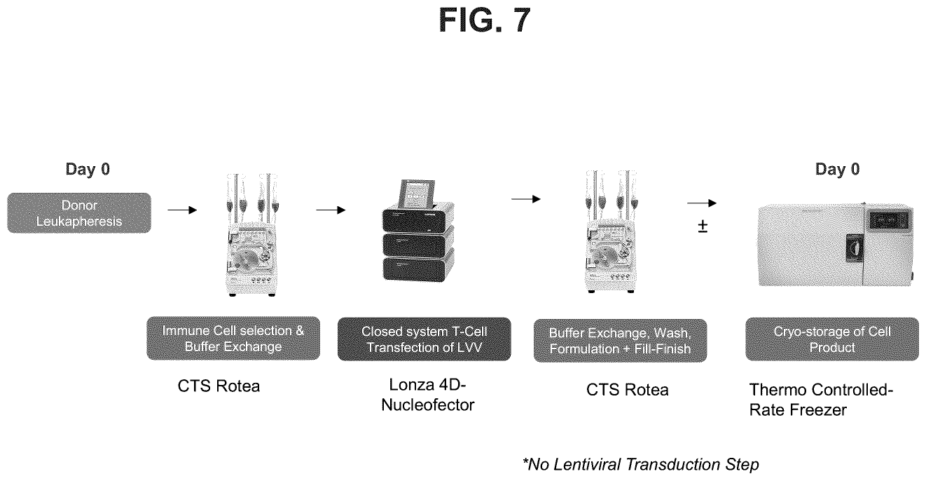

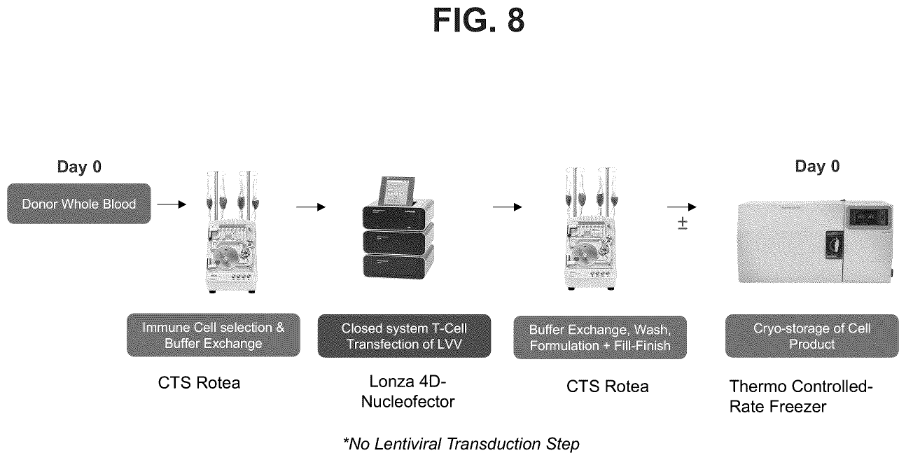

For example, the electroporation process can comprise lentiviral Nucleofection®. Ultrafast Electric CAR T cells were generated using the lentiviral Nucleofection Workflow shown in FIG. 7 and FIG. 8. The generation of Electric CAR T cells using this manufacturing process did not require cytokines and/or an ex vivo culture process. Moreover, nucleofection of CAR lentiviral vector resulted in effective CAR transgene integration into the T-cell genome. Further analyses also showed that the vector copy number per cell of the Electric CAR T cells were substantially similar to the copy number per cell of conventional CART cells (e.g., about 1-1.5 copies/cell). In addition, the mRNA encoding the CAR transgene (qRTPCR of WPRE sequence) was expressed within about 1 hour post nucleofection. Generally, electroporating the lentiviral vector significantly increased CAR T cell manufacturing efficiency using the process disclosed herein.

Third, the present disclosure shows for the first time that applying electricity to the cells up to 4 hours (e.g., up to 2 hours) before adding lentiviral viral vectors to the cells enhanced CAR expression by about 10-15% when compared to a traditional electroporation process (e.g., adding the expression vector to cell prior to electroporation). As such, the lentiviral vectors are used as cargo carriers in the shape of a particle; and physical transfection, such as electroporation, is used to deliver the lentiviral particle inside cells.

Table 2 and Table 3 summarize raw data from flow cytometry analyses of Electric CD19 CAR T cells produced by the methods disclosed and show that electroporation of a lentiviral vector in both unstimulated and stimulated primary human T cells at a very low multiplicity of infection (MOI) produced significant amounts of CAR-T cells. The transfection rates disclosed herein were very high when compared to conventional methods, which resulted in about 1 to about 3% CAR expression in T cells using traditional transduction methods (viral transduction only).

FIG. 11 shows that stimulated or unstimulated lentiviral vector transfected CD19 CAR T cells (CART19 Cells) efficiently killed target cells. Furthermore lentiviral vector Nucleofection® enhanced the percentage of CAR⁺ T cells (e.g., CD19 CAR T cells) by at least 10-fold when compared to lentiviral transduction (39.8% CAR⁺ T cells vs 3.5% CAR⁺ T cells) within the same time period.

Since Electric CAR T cells generated by the process disclosed herein are different from conventional CAR T cells at least because Electric CAR T cells were not activated ex vivo whereas conventional CART cells are produced in a 7-12 day process. In addition, the majority of unstimulated Electric CART cells (over 50%) maintained a non-activated or less differentiated phenotype when compared to Electric CAR T cells that are stimulated with e.g., CD3/CD28 Dynabeads® (Tables 4 and 5). The high percentage of less differentiated CAR T cells (e.g., naïve T cell population) is highly desirable and unexpected improvement because, less differentiated phenotype enhances CART cell persistence and potency in cancer patients. Furthermore, Electric CAR T cells were shown to be as effective as conventional CAR T cells at killing CD19⁺ Nalm6 cells within 48 hours following co-culture (Table 6). The Electric CAR T cells were effective at killing target cells at a effector:target ratio as low as 0.62:1 or less.

The novel manufacturing method described herein provided the most efficient CAR T cell immunotherapy known to date for several reasons. The Electric CAR T cell manufacturing process reduced the entire CAR T manufacturing process to a single day or at most 3-days if expansion (e.g., culturing) is desired. As such, the Electric CART cell manufacturing time is shortened from 12-15 days to about 1 day or at most 3 days. In addition, fewer cells were required. For example, traditional manufacturing processes require up to about 300 million cells, while the manufacturing process disclosed herein worked efficiently with about 3 million cells because the cells are general very fresh.

Lastly, the manufacturing process is cost effective because a batch of cells using conventional method can cost $1 million per batch, which is enough to treat about 8 patients. However, the methods disclosed herein will generate CAR T cell batches that are sufficient to infuse about 20 patients. As such, the methods disclosed herein double or triple the number of treated patients with the same cost and significantly reduce the CAR T cell cost per patient.

Accordingly, the manufacturing processes described herein provide an about 1 day or less (or in other aspects about 3 days or less, or other time period described herein) production of effective clinical-grade CAR- or TCR-engineered cells for immediate administration, which is an improvement over the manufacturing processes known in the art.

### II. Methods of Generating a Modified T Cell

The present disclosure provides quick and efficient manufacturing processes for engineering modified cells (e.g., immune effector, Electric CAR T cells) comprising a CAR, an exogenous TCR and/or an immune enhancing factor that improves the fitness of the engineered immune cells; compositions comprising the engineering modified cells, and methods of using the engineering modified cells for treating a disease, such as cancer, in a subject. The quick and efficient manufacturing methods of engineering immune cells disclosed herein provide engineered CAR T cells (i.e., electric) in less than 24 hours after transfection. The quick turn-around is made possible by a combination of at least three factors: (1) the use of fresh apheresis product; (2) the electroporation of lentiviral vectors at very low MOI into purified apheresis products (e.g., purified T cells or purified immune cells); and/or (3) newly engineered lentiviral vectors. CAR T cells engineered by the methods disclosed herein are called Electric CAR T cells. The CAR engineered by the process disclosed herein is referred to as an Electric CAR because electricity (e.g., electroporation) is used to drive the CAR coding vectors (e.g., lentiviral particles comprising a nucleic acid sequence encoding the CAR) into cells. In particular, rather than passively transducing cells with lentiviral vectors, lentiviral vectors are actively introduced into cells by electroporation.

A. Novel Electric CAR Manufacturing Platforms

One aspect of the present disclosure provides a method for manufacturing a population of engineered immune cells, the method comprising: (1) enriching a population of lymphocytes, a population immune cells, or a population of CD4⁺ and CD8⁺ cells from blood obtained from a subject; (2) admixing the population of lymphocytes, the population of immune cells, or the population of CD4⁺ and CD8⁺ cells with one or more buffer solutions; and (3) transfecting the population of lymphocytes, the population of immune cells, or the population of CD4⁺ and CD8⁺ cells with an effective dose of a modifying agent; thereby generating a population of modified lymphocytes, a population of modified immune cells, or a population of modified CD4⁺ and CD8⁺ cells.

In some embodiments, prior to the enriching step (e.g., enriching or selecting for a population of lymphocytes, a population of immune cells, or a population of CD4⁺ and CD8⁺ cells) the blood is separated into a plasma constituent, a mononuclear cell-containing layer, a platelet layer, and red blood cells by apheresis to produce an apheresis product selected from erythrocytapheresis, thrombapheresis, thrombocytapheresis, leukapheresis, stem cells, plasmapheresis, and plateletpheresis. In some embodiments, the apheresis product (e.g., the population of lymphocytes, the population of immune cells, or the population of CD4⁺ and CD8⁺ cells) is enriched by apheresis, elutriation, or gradient centrifugation.

In some embodiments, the apheresis sample is used at the point-of-care site in the methods of producing CAR T cells (i.e., Electric CAR) disclosed herein. Fresh apheresis samples are preferred because fewer immune cells are needed/required for optimal transfection using the methods disclosed herein. For example, traditional manufacturing processes require up to about 300 million cells, while the manufacturing processes disclosed herein work efficiently with about 3 million cells. This difference can be attributed to the freshness of the apheresis product.

In some embodiments, the apheresis sample (for example, a leukapheresis sample) is collected from a subject and shipped as a fresh product (e.g., a product that is not frozen) to a cell manufacturing facility. Desired cells (e.g., immune cells, CD4⁺ T cells and/or CD8⁺ T cells) are selected from the apheresis sample, for example, using a cell sorting machine such as a CliniMACS Prodigy® device. Enriched cells (e.g., immune cells, CD4⁺ T cells and/or CD8⁺ T cells) are then seeded for CART manufacturing using the methods described herein.

In some embodiments, the apheresis sample (for example, a leukapheresis sample) is collected from the subject and shipped as a frozen sample (for example, a cryopreserved sample) to a cell manufacturing facility. The frozen apheresis sample is then thawed, and desired cells (e.g., immune cells, CD4⁺ T cells and/or CD8⁺ T cells) are selected from the apheresis sample, for example, using a cell sorting machine such as a CliniMACSProdigy® device. Enriched cells (e.g., immune cells, CD4⁺ T cells and/or CD8⁺ T cells) are then seeded for CART manufacturing using the methods described herein. In some embodiments, at the end of the manufacturing process, the CAR T cells are harvested and cryopreserved and later thawed and administered to the subject. In some embodiments, enriched cells (e.g., CD4⁺ T cells and/or CD8⁺ T cells) undergo one or more rounds of freeze-thaw before being seeded for CAR T manufacturing.

In some embodiments, the apheresis sample (for example, a leukapheresis sample) is collected from the subject. Desired cells (e.g., immune cells, CD4⁺ T cells and/or CD8⁺ T cells) are selected from the apheresis sample, for example, using a cell sorting machine, such as CliniMACS Prodigy® device. Enriched cells (f e.g., immune cells, CD4⁺ T cells and/or CD8⁺ T cells) are then shipped as a frozen sample (e.g., a cryopreserved sample) to a cell manufacturing facility. Enriched cells (e.g., immune cells, CD4⁺ T cells and/or CD8⁺ T cells) are then later thawed and seeded for CART manufacturing using the methods described herein.

In some embodiments, after cells (for example, T cells) are seeded, one or more cytokines as well as one or more modifying agents (e.g., vectors encoding a CAR) are added to the cells. In that embodiment, the one or more cytokines can be selected from the group consisting of IL-2, IL-7, IL-15, het1L-15 (IL15/sIL-15Ra)), IL-21, or IL-6 (e.g., IL-6/sIL-6R)) After incubation for at least about 5-72 hours, the cells are harvested, washed and formulated for storage (e.g., cryopreservation) or administration.

One aspect of the present disclosure provides a method for manufacturing a population of engineered immune cells, the method comprising: enriching a population of lymphocytes, a population immune cells, or a population of CD4⁺ and CD8⁺ cells from blood obtained from a subject; admixing the population of lymphocytes, the population of immune cells or the population of CD4⁺ and CD8⁺ cells with one or more buffer solutions; transfecting the population of lymphocytes, the population of immune cells, or the population of CD4⁺ and CD8⁺ cells with an effective dose of a modifying agent; culturing and expanding the transfected population of lymphocytes, immune cells, or CD4⁺ and CD8⁺ cells; and harvesting the engineered lymphocytes, immune cells, or CD4⁺ and CD8⁺ cells; thereby generating a population of modified lymphocytes, a population of modified immune cells, or a population of modified CD4⁺ and CD8⁺ cells. In some embodiments, the population of immune cells or the population of CD4⁺ and CD8⁺ cells is not stimulated and/or activated prior to transfection. In some embodiments, the transfected cells are cultured and expanded in the presence of one or more stimulating agents described herein.

Another aspect of the present disclosure provides a method for manufacturing a population of engineered immune cells, the method comprising: (1) enriching a population of lymphocytes, a population of immune cells or a population of CD4⁺ and CD8⁺ cells from a donor leukapheresis; (2) admixing the population of lymphocytes, the population of immune cells or the population of CD4⁺ and CD8⁺ cells with one or more buffer solutions; and (3) transfecting the population of lymphocytes, the population of immune cells, or the population of CD4⁺ and CD8⁺ cells with an effective dose of a modifying agent; thereby generating a population of modified lymphocytes, a population of modified immune cells or a population of modified CD4⁺ and CD8⁺ cells.

Another aspect of the present disclosure provides a method for manufacturing a population of engineered immune cells, the method comprising: (1) enriching a population of lymphocytes, a population of immune cells or a population of CD4⁺ and CD8⁺ cells from a donor leukapheresis; (2) admixing the population of lymphocytes, the population of immune cells or the population of CD4⁺ and CD8⁺ cells with one or more buffer solutions; (3) transfecting the population of lymphocytes, the population of immune cells, or the population of CD4⁺ and CD8⁺ cells with an effective dose of a modifying agent; (4) culturing and expanding the transfected population of lymphocytes, immune cells, or CD4⁺ and CD8⁺ cells; and (5) harvesting the engineered lymphocytes, immune cells, or CD4⁺ and CD8⁺ cells; thereby generating a population of modified lymphocytes, a population of modified immune cells or a population of modified CD4⁺ and CD8⁺ cells. In some embodiments, the transfected cells are cultured and expanded in the presence of one or more stimulating agents described herein. In some embodiments, the population of lymphocytes, the population of immune cells or the population of CD4⁺ and CD8⁺ cells is not stimulated and/or activated prior to transfection.

In some embodiments, the apheresis product (e.g., the population of lymphocytes, the population of immune cells, or the population of eukaryotic donor cells) is selected from the group consisting of mononuclear cells, Lymphocytes rich cells, B lymphocytes, T lymphocytes, CD4⁺ T lymphocytes, CD8⁺ T lymphocytes, dendritic cells, monocytes, natural killer (NK) cells, natural killer T (NKT) cells, T-regulatory cells, CD4⁺ T-helper cells, CD8⁺ cytotoxic T lymphocytes (CTLs), CD62L⁺ cells, CD27⁺ cells, CCR7⁺ cells, CD45RO⁻ cells, CD45RA⁺ cells, neutrophils, basophils, eosinophils, megakaryocytes, stem cells, hematopoietic stem cells (HSCs), hematopoietic progenitor cells (HPCs), CD34⁺ cells, CD34⁺ peripheral blood stem cells, lymphokine-activated killer cells (LAKs), tumor infiltrating lymphocytes (TILs), mesenchymal stem cells, mast cells, a monocyte, a macrophage, a neutrophil, a basophil, an eosinophil, a dendritic cell, a megakaryocyte, and combinations thereof.

Another aspect of the present disclosure provides a method for manufacturing a population of engineered eukaryotic cells, the method comprising: (1) obtaining a population of eukaryotic donor cells (e.g., from a subject or a cell line); (2) admixing the population of eukaryotic donor cells with one or more buffer solutions; and (3) transfecting the population of eukaryotic donor cells with an effective dose of a modifying agent, thereby generating a population of modified eukaryotic donor cells. In some embodiments, the transfected cells are cultured and expanded in the presence of one or more stimulating agents.

Another aspect of the present disclosure provides a method for manufacturing a population of engineered eukaryotic cells, the method comprising: (1) obtaining a population of eukaryotic donor cells from a subject; (2) admixing the population of eukaryotic donor cells with one or more buffer solutions; (3) transfecting the population of eukaryotic donor cells with an effective dose of a modifying agent; (4) culturing and expanding the transfected population of eukaryotic donor cells; and (5) harvesting the engineered eukaryotic cells, thereby generating a population of modified eukaryotic donor cells. In some embodiments, the transfected cells are cultured and expanded in the presence of one or more stimulating agents. In some embodiments, the population of eukaryotic donor cells is not activated and/or stimulated prior to transfection.

In some embodiments, the methods disclosed herein can manufacture the population of modified lymphocytes, the population of modified immune cells, or the population of modified CD4⁺ and CD8⁺ cells expressing a modifying agent such as a CAR in about less than about 24 hours, in about 24 hours, or within about 24 hours. In some embodiments, the methods disclosed herein can manufacture the population of modified lymphocytes, the population of modified immune cells, or the population of modified CD4⁺ and CD8⁺ cells expressing a modifying agent such as a CAR within about 24 hours. In some embodiments, the methods disclosed herein can manufacture the population of modified lymphocytes, the population of modified immune cells, or the population of modified CD4⁺ and CD8⁺ cells expressing a modifying agent such as a CAR within about 48 hours or less. In some embodiments, the methods disclosed herein can manufacture the population of modified lymphocytes, the population of modified immune cells, or the population of modified CD4⁺ and CD8⁺ cells expressing a modifying agent such as a CAR within about 72 hours or less.

B. Sources of Immune Cells

The method for manufacturing a population of engineered immune cells disclosed herein comprises obtaining immune cells from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Target cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity (myeloid or lymphoid cells, including lymphocytes, typically T cells and/or NK cells). In some aspects, the cells are human cells. With reference to the subject to be treated, the cells may be allogeneic and/or autologous. The cells typically are primary cells, such as those isolated directly from a subject and/or isolated from a subject and frozen.

1. Immune Cells

In certain embodiments, the target cell is an immune cell, a T cell (e.g., a CD8⁺ T cell, a CD8⁺ naive T cell, central memory T cell, or effector memory T cell, a CD4⁺ T cell, a natural killer T cell (NKT cells), a regulatory T cell (Treg), a stem cell memory T cell, a lymphoid progenitor cell, a hematopoietic stem cell, a natural killer cell (NK cell) or a dendritic cell. In some embodiments, the cells are monocytes or granulocytes (e.g., myeloid cells, macrophages, neutrophils, dendritic cells, mast cells, eosinophils, and/or basophils).

In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. Among the sub-types and subpopulations of T cells and/or of CD4⁺ and/or of CD8⁺ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

2. Stem Cells

Other exemplary cells that can be engineered using the manufacturing processes of the present disclosure include stem cells, such as multipotent and pluripotent stem cells, including induced pluripotent stem cells (iPSCs). In an embodiment, the target cell is an induced pluripotent stem (iPS) cell or a cell derived from an iPS cell, e.g., an iPS cell generated from a subject. In some embodiments, the iPS cells are manipulated to alter (e.g., induce a mutation in) or to induce the expression of one or more target genes. In some embodiments, the iPS cells are differentiated into, a T cell, a CD8⁺ T cell (e.g., a CD8⁺ naive T cell, central memory T cell, or effector memory T cell), a CD4⁺ T cell, a stem cell memory T cell, a lymphoid progenitor cell or a hematopoietic stem cell.

3. Cell Isolation

In some embodiments, the manufacturing processes of the present disclosure includes isolating target cells (e.g., immune cells; the enriched apheresis product) from the subject, preparing, processing, optionally culturing, and/or transfecting them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition, in need of a cell therapy, or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells (e.g., primary human cells). The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids (e.g., blood, plasma, serum, cerebrospinal fluid, synovial fluid, urine and sweat), tissue and organ samples, and processed samples derived therefrom.

In certain aspects, the sample from which the immune cells are derived or isolated is blood, a blood-derived sample, or an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. In the context of cell therapy (e.g., adoptive cell therapy), samples may be from autologous and allogeneic sources.

In some embodiments, isolation of the cells includes one or more preparation and/or non-affinity based cell separation steps. In some embodiments, cells are washed, centrifuged, and/or incubated in the presence of one or more reagents (e.g. to remove unwanted components), enrich for desired components, lyse or remove cells sensitive to particular reagents. In some embodiments, cells are separated based on one or more property, such as density, adherent properties, size, sensitivity and/or resistance to particular components.

In some embodiments, cells from the circulating blood of a subject are obtained by apheresis. The samples, in certain aspects, contain lymphocytes, including T cells, monocytes, granulocytes, B cells, other nucleated white blood cells, red blood cells, and/or platelets, and in certain aspects contains cells other than red blood cells and platelets. In some embodiments, the blood cells collected from the subject are washed to remove the plasma fraction and/or to place the cells in an appropriate buffer or media for subsequent processing steps. In some embodiments, the cells are washed with phosphate buffered saline (PBS). In some embodiments, a washing step is accomplished by tangential flow filtration (TFF) according to the manufacturer's instructions. In some embodiments, the cells are resuspended in a variety of biocompatible buffers after washing. In some embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or another saline solution with or without buffer. In some embodiments, the undesirable components of the apheresis sample may be removed and the cells directly resuspended in culture media.

In some embodiments of the manufacturing processes described herein, cells are obtained from the circulating blood of a subject by apheresis or leukapheresis using an extracorporeal apheresis system. In some embodiments, cell isolation and transfection are performed the same day.

4. Extracorporeal Apheresis

In some embodiments of the manufacturing processes described herein, cells are collected using standard apheresis equipment, such as Cobe® Spectra, Spectra Optia®, Fenwal™ Amicus® or equivalent. In some embodiments, cells from the circulating blood of a subject are obtained by erythrocytapheresis, thrombapheresis, thrombocytapheresis, Leukapheresis, stem cells harvesting, plasmapheresis, or plateletpheresis. The leukapheresis process typically yielded approximately 200-400 mL of apheresis product from a patient (i.e., a subject). The apheresis product is subjected to the manufacturing process on-site (e.g., point-of-care.

In some embodiments, the enriched apheresis product is a “leukapheresis” product. As used herein, the term “Leukapheresis” refers to the bulk mononuclear cells present in the blood, namely the separation and collection of leukocytes (WBC) from plasma and red blood cells.

In some embodiments, the enriched apheresis product comprises about 5% to about 25% of the total peripheral blood mononuclear cell component. In some embodiments, the enriched apheresis product is a population of lymphoid cells or a lymphoid cell. In this embodiment, the lymphoid cell is selected from the group consisting of a T cell, a B cell, a natural killer (NK) cell, a CD8⁺ T cell, a CD4⁺ T cell, a cytotoxic T lymphocyte, a regulatory T cell, and any combination thereof.

In some embodiments, the enriched apheresis product is a population of myeloid cells or a myeloid cell. In this embodiment, the myeloid cell can be selected from a monocyte, a macrophage, a neutrophil, a basophil, an eosinophil, a dendritic cell, a megakaryocyte, and any combination thereof.

In some embodiments, the enriched apheresis product has a predetermined volume and/or a predetermined hematocrit regardless of the number of mononuclear cell collection cycles executed by the Apheresis system and/or the number of pre-products used to produce the enriched apheresis product.

In some embodiments, the predetermined volume is about 120 ml to about 400 mL. In some embodiments, the predetermined volume is about 120 ml, about 150 ml, about 175 ml, about 180 ml, about 200 ml, about 225 ml, about 250 ml, about 275 ml, about 300 ml, about 325 ml, about 350 ml, about 375 ml, or about 400 ml or less. In some embodiments, the apheresis is configured with a specific target yield of mononuclear cells to be collected and treated. The specific target yield of mononuclear cells to be collected and treated may be assessed by the apheresis system and/or by entering the subject's mononuclear cell pre-count. Based on the target mononuclear yield and the number of mononuclear cells collected during each mononuclear collection cycle, the controller of the apheresis system may determine the number of mononuclear collection cycles to execute. As an example, in some embodiments, if the target mononuclear yield is about 5×10⁹ mononuclear cells, the apheresis system will collect about 1×10⁹ mononuclear cells per mononuclear collection cycle, then the controller will determine that it is appropriate to execute the mononuclear collection cycle five times.

In some embodiments, the target mononuclear yield is at least about 0.7×10⁷, at least about 0.8×10⁷, at least about 0.9×10⁷, at least about 1×10⁷, at least about 2×10⁷, at least about 4×10⁷, at least about 6×10⁷, at least about 8×10⁷, at least about 1×10⁸, or at least about 5×10⁸ cells/mL. In some embodiments, the target mononuclear yield is from about 0.5×10⁶ cells/mL to about 4×10⁶ cells/mL. In some embodiments, target mononuclear yield is from about 0.5×10⁶ cells/mL to about 1×10⁸ cells/mL. In some embodiments, target mononuclear yield is from about 4.0×10⁶ cells/mL to about 1×10⁸ cells/mL.

In some embodiments, the predetermined hematocrit is about 0% to about 10%. In another embodiment, the predetermined hematocrit is about 2%. In some embodiments, the predetermined volume is approximately 200 mL and the predetermined hematocrit is approximately 2%. The predetermined volume and/or the predetermined hematocrit may vary without departing from the scope of the present disclosure.

5. Cell Enrichment

In some embodiments, the manufacturing processes disclosed herein comprise a selection of specific cells to improve the enrichment of the desired immune effector cells suitable for CAR expression. Systems or devices used for cell enrichment and purification purposes include, for example, the BAXTER ISOLEX 300I™ and the Miltenyi CLINIMACS™, which enrich peripheral blood progenitor cells (PBPC) based on a specific ligand on the cells' surface (e.g., CD34, or CD133).

In some embodiments, the selection comprises a positive selection, for example, selection for the desired immune effector cells. In some embodiments, the selection comprises a negative selection, for example, selection for unwanted cells, for example, removal of unwanted cells. In some embodiments, the positive or negative selection methods described herein are performed under flow conditions by using a flow-through device or a cell processing system, to further enrich a preparation of cells for desired immune effector cells. Negative T cell selection via removal of unwanted cells with CD19, CD14 and CD26 Miltenyi beads in combination with column technology (CliniMACS® System, CliniMACS® Plus, or CliniMACS Prodigy®). Positive T cell selection with a combination of CD4 and CD8 Miltenyi beads and column technology (CliniMACS® System, CliniMACS® Plus, or CliniMACSProdigy®) can be used. Alternatively, column-free technology with releasable CD3 beads (GE Healthcare) can be used. In addition, bead-free technologies such as ThermoGenesis X-series devices can be utilized as well. Additional exemplary cell separation and debeading methods are known to those of skills in the art, for example as shown in WO 2017/117112.

In some embodiments, the enriched apheresis product is enriched for one or more target cell types selected from the group consisting of B lymphocytes, T lymphocytes, CD4 and CD8 T lymphocytes, dendritic cells, monocytes, natural killer (NK) cells, NKT cells, T-regulatory cells, CD4 T-helper cells, CD8 cytotoxic T lymphocytes (CTLs), NKT cells, neutrophils, basophils, eosinophils, megakaryocytes, hematopoietic stem cells (HSCs), hematopoietic progenitor cells (HPCs), lymphokine-activated killer cells (LAKs), tumor infiltrating lymphocytes (TILs), mesenchymal stem cells, mast cells, subsets of such cells, and combinations thereof.

In some embodiments, the enriched apheresis product is enriched for one or more target lymphocyte or myeloid cell populations. In some embodiments, the enriched apheresis product is enriched for a lymphoid cell selected from the group consisting of a T cell, a B cell, a natural killer (NK) cell, a CD8⁺ T cell, a CD4⁺ T cell, a cytotoxic T lymphocyte, a regulatory T cell, and any combination thereof.

In some embodiments, the enriched apheresis product is enriched for a myeloid cell selected from a monocyte, a macrophage, a neutrophil, a basophil, an eosinophil, a dendritic cell, a megakaryocyte, and any combination thereof.

In some embodiments, one or more markers, one or more “cell-surface determinants” or “cell-surface markers” are used to enrich for a target cell population. In some embodiments, one or more markers or cell-surface determinants are selected from: CD19 and/or CD20 for B cells; CD3, CD56⁻, CD4, and/or CD8 for T cells; CD25 and/or CD69 for Activated and/or regulatory T cells; CD1c, CD83, CD141, CD209, MEW II, and/or CD11c for Dendritic cells, CD3−, CD16 and/or CD56 for NK cells; CD34, CD90, and/or CD135 for hematopoietic stem or progenitor cells; CD11b, CD68, CD163, and/or CD33 for macrophages; CD14, CD16, and/or CD64 for monocytes, CD15, CD16, and/or CD49d⁻ for neutrophils; 2D7 antigen, CD117−, CD123, CD203c, and/or FcεRIa for basophils; or CD11b, CD193, EMR1, and/or Siglec-8 for eosinophils.

Techniques for enriching enriched apheresis product are known to a person of skill in the art and include but are not limited to magnetic separation, filtration, immunoaffinity separation, gravitation separation, density gradient separation, elutriation, and any combinations thereof. The cell separation module can employ any of these or other methods known in the art for further enriching for and/or obtaining a target population of nucleated blood cells from a patient. For example, binding to one or more selective or affinity agents, such as antibodies attached to degradable buoyant beads or magnetic beads or microbubbles, can be used to enrich for a particular target cell type or class, following the cell separation.

In some embodiments, magnetic beads coated with antibodies against one or more specific cell-surface antigens are used to enrich for target cell populations from an enriched apheresis product. This causes a cell expressing the target antigen to attach to the magnetic beads. When exposed to a strong magnetic field, the cells attached to the beads (expressing the cell-surface marker) stay on the column or sample tube, while other cells (not expressing the cell-surface marker) flow through or remain in suspension. Using this method, cells can be selected positively or negatively, or using a combination of positive and negative selection, with respect to the particular cell-surface markers. In some embodiments, cells are still coupled with the microbead-bound antibodies during transfection. In some embodiments, cells are decoupled from the microbead-bound antibodies before transfection.

In some embodiments, a target cell is enriched using one or more methods known in the art including, but not limited to antigen capture. In some embodiments, the antigen capture is selected from the group consisting of filters, beads, magnetic beads, fluorescence-activated cell sorting, microfluidics, solid support affinity, acoustics, bioluminescence, antibody tagging, and enzyme substrate. In some embodiments, a target cell is enriched using a suitable solid support selected from the group consisting of ferromagnetic and density modified particles. In some embodiments, solid supports comprise affinity molecules, such as antibody domains that bind a given cell-surface marker can be obtained, for instance from Miltenyi Biotec and Dynal. Methods that can be used for the release of the captured cells include, competition with excess ligand, enzymatic digestion, change in pH, change in ionic strength, removal of magnetic field, and/or physical agitation.

In some embodiments, the isolation methods include the separation of different cell types based on the expression or presence in the cell of one or more specific molecules, such as surface markers, surface proteins, intracellular markers, or nucleic acids. In some embodiments, any known method for separation based on such markers may be used. In some embodiments, the separation is affinity- or immunoaffinity-based separation. For example, the isolation may include separation of cells and cell populations based on the cells' expression or expression level of one or more markers. Typically cell surface markers are incubated with an antibody or a binding partner that specifically binds to the markers. This incubation step is followed by washing steps and purification of cells having bound the antibody or binding partner from those cells that are not bound by the antibody or the binding partner. Such purification steps can be based on positive selection, in which the cells having bound the reagents are retained for further use, and/or negative selection, in which the cells having not bound to the antibody or binding partner are retained. In some examples, both fractions are retained for further use.

In some embodiment, a negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, a positive selection of or enrichment for cells of a particular type (e.g. those expressing a marker), may increase the number or percentage of such cells, but need not result in a complete absence of cells not expressing the marker. Likewise, a negative selection, removal, or depletion of cells of a particular type (e.g., those expressing a marker) may decrease the number or percentage of such cells, but need not result in a complete removal of all such cells. In certain exemplary embodiments, multiple rounds of separation steps may be carried out, where the positively or negatively selected fraction from one step is subjected to another separation step, such as a subsequent positive or negative selection. In certain exemplary embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

Enrichment of a T cell population by negative selection can be accomplished using a combination of antibodies directed to surface markers unique to the negatively selected cells. An exemplary method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surfaces (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than about 100 million cells/ml is used. In a further embodiment, a concentration of cells of about 1, about 2, about 3, about 4, about 5, about 6, about 7, about 8, about 9, about 10, about 15, about 20, about 25, about 30, about 35, about 40, about 45, or about 50 million cells/ml is used. In yet another embodiment, a concentration of cells from about 75, about 80, about 85, about 90, about 95, or about 100 million cells/ml is used. In further embodiments, concentrations of about 125 or about 150 million cells/ml can be used. Using high concentrations can result in increased cell yield, cell activation, and cell expansion.

In some embodiments, different cell types are enriched using a cell sorting machine such as a CliniMACS Prodigy®. For example, T cells (for example, CD4⁺ T cells and/or CD8⁺ T cells) may be selected from an apheresis product using a cell sorting machine such as a CliniMACS Prodigy® device. The selected T cells (for example, CD4⁺ T cells and/or CD8⁺ T cells) are then washed and transfected to manufacture engineered T cells as described herein.

In some embodiments, one or more of T cell populations are enriched for or depleted of cells that are positive for (marker⁺) or express high levels (marker^{high}) of one or more particular markers, such as surface markers, or that are negative for (marker⁻) or express relatively low levels (marker^{low}) of one or more markers. For example, in certain aspects, specific subpopulations of T cells, such as cells positive or expressing high levels of one or more surface markers (e.g., CD28⁺, CD62L⁺, CCR7⁺, CD27⁺, CD127⁺, CD4⁺, CD8⁺, CD45RA⁺, and/or CD45RO⁺ T cells) are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8⁺ cells or the T cells, e.g., CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some embodiments of the methods disclosed herein, the enriching step of the apheresis product comprises CD25⁺ cell depletion. In some embodiments of the methods disclosed herein, the enriching step of the apheresis product does not comprise CD25⁺ cell depletion. See e.g., WO 2016/109410. CD25⁺ depletion can enhance the lentiviral transduction efficiency, which can ultimately improve the therapeutic effect of CAR T therapy. However, this step may not be critical for manufacturing the Electric CAR T cells described herein.

In certain exemplary embodiments, CD8⁺ T cells are enriched for cells positive for CD45RO (or negative for CD45RA) and for CD62L. For example, CD3⁺, CD28⁺ T cells can be positively selected using CD3/CD28 conjugated magnetic beads (e.g., Dynabeads® M-450 CD3/CD28 T Cell Expander).

In some embodiments, T cells are separated from a peripheral blood mononuclear cell (PBMC) sample by negative selection of markers expressed on non-T cells, such as B cells, monocytes, or other white blood cells, such as CD14. In certain aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations. In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in certain aspects is particularly robust in such sub-populations.

In some embodiments, combining TCM-enriched CD8⁺ T cells and CD4⁺ T cells further enhances efficacy. In some embodiments, memory T cells are present in both CD62L⁺ and CD62L⁻ subsets of CD8⁺ peripheral blood lymphocytes. PBMC can be enriched for or depleted of CD62L-CD8⁺ and/or CD62L⁺ CD8⁺ fractions, such as using anti-CD8 and anti-CD62L antibodies. In some embodiments, a CD4⁺ T cell population and/or a CD8⁺ T population is enriched for central memory (TCM) cells. In some embodiments, the enrichment for central memory T (TCM) cells is based on positive or high surface expression of CD45RO, CD62L, CCR7, CD28, CD8, and/or CD127. In some embodiment, the enrichment may be based on negative selection for cells expressing or highly expressing CD45RA and/or granzyme B. In some embodiments, isolation of a CD8⁺ population enriched for TCM cells is carried out by depletion of cells expressing CD4, CD14, CD45RA, and positive selection or enrichment for cells expressing CD62L. In some embodiment, enrichment for central memory T (TCM) cells is carried out starting with a negative fraction of cells selected based on CD4 expression, which is subjected to a negative selection based on expression of CD14 and CD45RA, and a positive selection based on CD62L. Such selections in certain aspects are carried out simultaneously and in other aspects are carried out sequentially, in either order. In some embodiment, the same CD4 expression-based selection step used in preparing the CD8⁺ cell population or subpopulation, also is used to generate the CD4⁺ cell population or sub-population, such that both the positive and negative fractions from the CD4-based separation are retained and used in subsequent steps of the methods, optionally following one or more further positive or negative selection steps.

CD4⁺ T helper cells are sorted into naive, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L- and CD45RO. In one example, to enrich for CD4⁺ cells by negative selection, a monoclonal antibody cocktail typically includes antibodies to CD14, CD20, CD11b, CD16, HLA-DR, and CDS. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent. The ligand may be capable of activating an intracellular signaling domain of a TCR complex. In some embodiments, the agent turns on or initiates TCR/CD3 intracellular signaling cascade in a T cell. Such agents can include antibodies, such as those specific for a TCR component (, e.g., anti-CD3, anti-CD28), costimulatory receptor and/or one or more cytokines. The agent may be bound to solid support such as a bead. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/ml. In some embodiments, the stimulating agents include IL-7 and/or IL-15. In some embodiments, the stimulating agents include IL-2, IL-7, and/or IL-15. In some embodiments, the stimulating agents include IL-2, IL-15, and/or IL-15Ra. In some embodiments, the stimulating agents include IL-2 and/or heterodimeric IL-15 (i.e., a polypeptide comprising IL-15 and IL-15 receptor alpha chains).

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques. The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

C. Pre-Transfection Activation

One aspect of the method disclosed herein does not require stimulating and/or activating the enriched apheresis product (e.g., the population of immune cells, the population of CD4⁺ and CD8⁺ cells, or the population of eukaryotic donor cells) with one or more stimulating agents prior to the transfecting step. However,

in some embodiments, the method may further comprise stimulating and/or activating the enriched apheresis product (e.g., the population of immune cells, the population of CD4⁺ and CD8⁺ cells, or the population of eukaryotic donor cells) with one or more stimulating agents prior to the transfecting step. The stimulating agents described herein (e.g., CD3, CD28, cytokines and/or growth factors) can promote efficient transduction and/or electroporation of primary human immune cells (e.g., T cells) and supplementing the culture media with a cytokine selected from IL-7, IL-15, IL-15Ra, IL-7 and IL-15 and/or heterodimeric IL-15 (i.e., a polypeptide comprising IL-15 and IL-15 receptor alpha chains) dramatically can enhance the expansion of transfected cell. Moreover, the stimulation and/or activation pre- and/or post-transfection can preserve undifferentiated T cells during CART manufacturing, which can enhance the longevity of manufactured T cells, thereby improving the therapeutic efficacy of the CART therapy.

In some embodiments, the one or more stimulating agents are selected from the group consisting of agonistic antibodies, cytokines, recombinant costimulatory molecules, anti-CD3 antibodies or fragments thereof, anti-CD28 antibodies or fragments, small drug inhibitors, and/or a combination thereof. In some embodiments, the one or more stimulating agents are anti-CD3 and anti-CD28 antibodies or fragments thereof. In some embodiments, the one or more stimulating agents are anti-CD3 and anti-CD28 antibodies or fragments thereof and one or more cytokines.

1. CD3/TCR Complex

In some embodiments, the enriched apheresis product (e.g., the population of immune cells, the population of CD4⁺ and CD8⁺ cells, or the population of eukaryotic donor cells) is stimulated and/or activated with an agent that stimulates a CD3/TCR complex and/or an agent that stimulates a costimulatory molecule and/or growth factor receptor on the surface of the cells. In some embodiments, the agent that stimulates a CD3/TCR complex is an agent that stimulates CD3. In some embodiments, the agent that stimulates a CD3/TCR complex is chosen from an antibody (for example, a single-domain antibody, a heavy chain variable domain antibody, a peptibody, a Fab fragment, or a scFv), a small molecule, or a ligand (for example, a naturally-existing, recombinant, or chimeric ligand).

In some embodiments, the agent that stimulates a CD3/TCR complex does not comprise a bead. In some embodiments, the agent that stimulates a costimulatory molecule and/or growth factor receptor does not comprise a bead. In some embodiments, the agent that stimulates a CD3/TCR complex comprises an anti-CD3 antibody. In some embodiments, the agent that stimulates a CD3/TCR complex comprises an anti-CD3 antibody covalently attached to a colloidal polymeric nanomatrix. In some embodiments, the agent that stimulates CD3 comprises one or more of a CD3 or TCR antigen binding domain, (e.g., an anti-CD3 or anti-TCR antibody or an antibody fragment) comprising one or more CDRs, heavy chain, and/or light chain thereof as known to those of skilled in the art.

In some embodiments, the agent that stimulates a CD3/TCR complex and the agent that stimulates a costimulatory molecule and/or growth factor receptor comprise T Cell Trans Act™. In some embodiments, the agent that stimulates a CD3/TCR complex and the agent that stimulates a costimulatory molecule and/or growth factor receptor are comprised in a multispecific binding molecule. In some embodiments, the multispecific binding molecule comprises a CD3 antigen binding domain and a CD28 or CD2 antigen binding domain. In some embodiments, the multispecific binding molecules comprise one or more heavy and/or light chains. In some embodiments, the multispecific binding molecule comprises a bispecific antibody. In some embodiments, one or more of the plurality of bispecific antibodies are conjugated together into a multimer.

2. Costimulatory Molecule

) In some embodiments, the agent that stimulates a costimulatory molecule and/or growth factor receptor is an agent that stimulates CD28, ICOS, CD27, HVEM, LIGHT, CD40, 4-1BB, 0X40, DR3, GITR, CD30, TIN/11, CD2, CD226, or any combination thereof. In some embodiments, the agent that stimulates a costimulatory molecule and/or growth factor receptor is an agent that stimulates CD28. In some embodiments, the agent that stimulates a costimulatory molecule and/or growth factor receptor is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand).

In some embodiments, the agent that stimulates a costimulatory molecule and/or growth factor receptor comprises an anti-CD28 antibody. In some embodiments, the agent that stimulates a costimulatory molecule and/or growth factor receptor comprises an anti-CD28 antibody covalently attached to a colloidal polymeric nanomatrix. In some embodiments, the agent that stimulates a costimulatory molecule and/or growth factor receptor is an agent that stimulates CD28, ICOS, CD27, CD25, 4-1BB, IL6RA, IL6RB, or CD2. In some embodiments, the agent that stimulates a costimulatory molecule and/or growth factor receptor comprises one or more of a CD28, ICOS, CD27, CD25, 4-1BB, IL6RB, and/or CD2 antigen binding domain. For example, the agent may be n an anti-CD28, anti-ICOS, anti-CD27, anti-CD25, anti-4-1BB, anti-IL6RA, anti-IL6RB, anti-CD2 antibody, or an antibody fragment comprising one or more CDRs, heavy chain, and/or light chain thereof as known to those of skilled in the art.

In some embodiments, prior to the transfection step, the enriched apheresis product (e.g., the population of lymphocytes, the population of immune cells, the population of CD4⁺ and CD8⁺ cells, or the population of eukaryotic donor cells) is stimulated and/or activated in vitro with an agent that stimulates a CD3/TCR complex (for example, an anti-CD3 antibody) and/or an agent that stimulates a costimulatory molecule and/or growth factor receptor on the surface of the cells (e.g., an anti-CD28 antibody). In some embodiments, the enriched apheresis product (e.g., the population of lymphocytes, the population of immune cells, the population of CD4⁺ and CD8⁺ cells, or the population of eukaryotic donor cells) can be stimulated and/or activated for about less or equal to about 1 hour, about 1.5 hours, about 2 hours, about 2.5 hours, about 3 hours, about 3.5 hours, about 4 hours, about 4.5 hours, or about 5 hours.

In some embodiments, the enriched apheresis product (e.g., the population of lymphocytes, the population of immune cells, the population of CD4⁺ and CD8⁺ cells, or the population of eukaryotic donor cells) is stimulated and/or activated in vitro with an agent that stimulates a CD3/TCR complex (e.g., an anti-CD3 antibody) and/or an agent that stimulates a costimulatory molecule and/or growth factor receptor on the surface of the cells (e.g., an anti-CD28 antibody) for about 20 hours, about 21 hours, about 22 hours, about 23 hours, about 24 hours, about 25 hours, about 26 hours, about 27 hours, or about 28 hours.

In some embodiments, the agent that stimulates a CD3/TCR complex and the agent that stimulates a costimulatory molecule and/or growth factor receptor are comprised in a multispecific binding molecule. The multispecific binding molecules can comprise an agent that stimulates a CD3/TCR complex and an agent that stimulates a costimulatory molecule and/or growth factor receptor. For example, the multispecific binding molecule can comprise a CD3 antigen binding domain and one or more of a CD28, ICOS, CD27, CD25, 4-IBB, IL6RA, IL6RB, and/or CD2 antigen binding domain. In some embodiments, the multispecific binding molecule comprises a CD3 antigen binding domain and a CD28 or CD2 antigen binding domain.

3. Cytokines

In some embodiments, the one or more stimulating agents are cytokines selected from the group consisting of Interleukin-2 (IL-2), Interleukin-3 (IL-3), Interleukin-6 (IL-6), Interleukin-7 (IL-7), Interleukin-7 receptor (IL-7R), Interleukin-11 (IL-11), Interleukin-12 (IL-12), Interleukin-15 (IL-15), Interleukin-15 receptor (IL-15R), heterodimeric IL-15 (i.e., a polypeptide comprising IL-15 and IL-15 receptor alpha chains), Interleukin-18 (IL-18), Interleukin-18 receptor (IL-18R), Interleukin-21 (IL-21), granulocyte macrophage colony stimulating factor, alpha, beta or gamma interferon, erythropoietin, and any combination thereof.

The cytokine may be selected from IL-2, IL-7, IL-6, IL-15, IL-15Ra, heterodimeric IL-15 (i.e., a polypeptide comprising IL-15 and IL-15 receptor alpha chains; hetIL-15), or IL-21. IL-2 is the most frequently used cytokine for generating lymphocytes for adoptive immunotherapy. IL-2 promotes T cell survival and expansion, enhances tumor-killing ability of T cells. IL-2 significantly increased the accumulation of CAR-T cells and their cytotoxicity ability, but IL-2 exposed CAR-T cells presented inferior antitumor immunity in vivo following adoptive transfer. IL-2 exposed CAR-T cells also displayed a relative mature phenotype with low expression of CD62L, CCR7, CD27 and CD28, which are less persistent in vivo. Adoptive transfer of less differentiated T cells correlates with superior tumor regression, which supports the finding that IL-2 exposed CAR-T cells are less effective than other group (Gattinoni et al., Nat Med, 2011, 17: 1290-7; and Markley et al., Blood, 2010, 115:3508-19).

IL-15 presented similar performance of stimulating CAR-T cell expansion and tumor-lysis function as IL-2, and showed better antitumor immunity in animal models. In addition, IL-15 induced a less differentiated phenotype (higher expression of CD27 and CD28). Therefore, IL-15 can support the persistence of CAR-T cells in vivo. IL-7 similarly promoted CAR-T cell expansion in vitro.

IL-7 also induced higher level of CD62L expression and exhibited the highest proportion of CAR-Tscm cells in an antigen-free circumstance. Ex vivo exposure of T cells or CAR T cells to IL-7 without antigen challenge enhanced the antitumor efficacy of the CAR-T cells. However, IL-7 exposed CAR-T cells did not result in better in vivo antitumor efficacy when compared to IL-2. IL-7's efficacy was also inferior to IL-15 due to the less expansion of CAR-T cells under antigen challenge. The combination of IL-7 and IL-15 promote the generation of Tscm, which is beneficial for producing more “young” CAR-T cells. Thus, combining IL-7 and IL-15 can promote CAR-T cell expansion and induce T cell phenotypes that are most efficacious for therapeutic treatment.

IL-21 can induce the expansion of less differentiated CAR-T cells, with a phenotype of high expression of CD62L, CCR7, CD27 and CD28, even under the circumstance of antigen challenge. Therefore, IL-21 exposed CAR-T cells showed best persistence in animal models and IL-21 injection in vivo, and also presented a better efficacy in promoting tumor eradication than other cytokine groups except IL-15. See e.g., WO 2016/109410.

Accordingly, in some embodiments, the cytokine can also be selected from IL-15 and IL-7; IL-7 and IL-21; IL-7 and IL-2; IL-15 and IL-2; IL-7, IL-15, and IL-21; IL-15 and IL-15Ra; or IL-7, IL-15 and IL-15Ra. In some embodiments, the cytokine is IL-2. In some embodiments, the cytokine is IL-15 (e.g., hetIL-15 (IL15/sIL-15Ra)). In some embodiments, the cytokine is IL-6 (e.g., IL-6/sIL-6Ra). In some embodiments, the cytokine is IL-7. In some embodiments, the cytokine is IL-7 and IL-15

In some embodiments, the enriched apheresis product (e.g., the population of lymphocytes, the population of immune cells, the population of CD4⁺ and CD8⁺ cells, or the population of eukaryotic donor cells) is stimulated and/or activated with about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, or about 300 U/ml of IL-2 (or any amount in between these values). In some embodiments, the enriched apheresis product (e.g., the population of immune cells, the population of CD4⁺ and CD8⁺ cells, or the population of eukaryotic donor cells) is stimulated/and activated with about 1, about 2, about 3, about 4, about 5, about 6, about 7, about 8, about 9, about 10, about 11, about 12, about 13, about 14, about 15, about 16, about 17, about 18, about 19, or about 20 ng/ml of IL-7 (or any amount in between these values). In some embodiments, the enriched apheresis product (e.g., the population of immune cells, the population of CD4⁺ and CD8⁺ cells, or the population of eukaryotic donor cells) is stimulated and/or activated with about 1, about 2, about 3, about 4, about 5, about 6, about 7, about 8, about 9, about 10, about 11, about 12, about 13, about 14, about 15, about 16, about 17, about 18, about 19, or about 20 ng/ml of IL-15 (or any amount in between these values). The cytokine stimulation preserves or increases the undifferentiated phenotype of T cells during the CAR T (Electric CAR T cell) manufacturing disclosed herein and generate CAR T cells (Electric CAR T cells) that persist longer in a subject following administration. Supplementing the culture media with a cytokine selected from IL-7, IL-15, IL-15Ra, IL-7 and IL-15 and/or heterodimeric IL-15 (i.e., a polypeptide comprising IL-15 and IL-15 receptor alpha chains) dramatically enhances transduced cell expansion by at least 200 fold over a 14 day period. See e.g., WO 2016/109410.

4. Nanostructure

In some embodiments, the one or more stimulating agents are conjugated to a bead or a nanostructure. In some embodiments, the nanostructure is a nanomatrix. The nanomatrix can comprise a matrix of mobile polymer chains, and anti-CD3 and anti-CD28 antibodies or fragments thereof. The nanomatrix can be about 1 to about 500 nm in size (or any size in between these two values). In some embodiments, the one or more stimulating agents are a nanomatrix and one or more cytokines described herein.

In some embodiments, the nanomatrix can comprise a polymeric, biodegradable or biocompatible inert material. An inert material may be non-toxic to cells. In some embodiments, the nanomatrix can be composed of hydrophilic polymer chains, which obtain maximal mobility in aqueous solution due to hydration of the chains. In some embodiments, the mobile nanomatrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum or alginate. Other polymers may include polyesters, polyethers, poly acrylates, polyacrylamides, polyamines, polyethylene imines, polyquaternium polymers, polyphosphazenes, polyvinylalcohols, polyvinylacetates, polyvinylpyrrolidones, block copolymers, or polyurethanes. In some embodiments, the mobile nanomatrix is a polymer of dextran.

Another aspect of the present disclosure provides a method for manufacturing a population of engineered eukaryotic cells, the method comprising: (1) obtaining a population of eukaryotic donor cells from a subject; (2) admixing the population of eukaryotic donor cells with one or more buffer solutions; (3) stimulating the population of eukaryotic donor cells with one or more stimulating agents; (4) transfecting the population of stimulated eukaryotic donor cells with an effective dose of a modifying agent; (5) culturing and expanding the transfected population of eukaryotic donor cells, and (6) harvesting the engineered eukaryotic cells, thereby generating a population of modified eukaryotic donor cells. In some embodiments, the transfected cells are cultured and expanded in the presence of one or more stimulating agents.

Another aspect of the present disclosure provides a method for manufacturing a population of engineered immune cells, the method comprising: (1) enriching a population of lymphocytes, a population of immune cells or a population of CD4⁺ and CD8⁺ cells from a donor leukapheresis; (2) admixing the population of lymphocytes, the population of immune cells or the population of CD4⁺ and CD8⁺ cells with one or more buffer solutions; (3) stimulating the population of eukaryotic donor cells with one or more stimulating agents; (4) transfecting the population of lymphocytes, the population of immune cells, or the population of CD4⁺ and CD8⁺ cells with an effective dose of a modifying agent; (5) culturing and expanding the transfected population of lymphocytes, immune cells, or CD4⁺ and CD8⁺ cells; and (6) harvesting the engineered lymphocytes, immune cells, or CD4⁺ and CD8⁺ cells; thereby generating a population of modified lymphocytes, a population of modified immune cells or a population of modified CD4⁺ and CD8⁺ cells. In some embodiments, the transfected cells are cultured and expanded in the presence of one or more stimulating agents.

One aspect of the present disclosure provides a method for manufacturing a population of engineered immune cells, the method comprising: (1) enriching a population of lymphocytes, a population immune cells, or a population of CD4⁺ and CD8⁺ cells from blood obtained from a subject; (2) admixing the population of lymphocytes, the population of immune cells or the population of CD4⁺ and CD8⁺ cells with one or more buffer solutions; (3) stimulating the population of lymphocytes, the population of immune cells or the population of CD4⁺ and CD8⁺ cells with one or more stimulating agents; (4) transfecting the population of lymphocytes, the population of immune cells, or the population of CD4⁺ and CD8⁺ cells with an effective dose of a modifying agent; (5) culturing and expanding the transfected population of lymphocytes, immune cells, or CD4⁺ and CD8⁺ cells; and (6) harvesting the engineered lymphocytes, immune cells, or CD4⁺ and CD8⁺ cells; thereby generating a population of modified lymphocytes, a population of modified immune cells, or a population of modified CD4⁺ and CD8⁺ cells. In some embodiments, the transfected cells are cultured and expanded in the presence of one or more stimulating agents described herein.

In some embodiments, the enriched apheresis product (e.g., the population of lymphocytes, the population of immune cells, the population of CD4⁺ and CD8⁺ cells, or the population of eukaryotic donor cells) is stimulated and/or activated with anti-CD3 and anti-CD28 antibodies for, about 12 hours, in the presence of a cytokine described herein followed by transfection with a modifying agent (e.g., transduction and/or electroporation with a vector, or a lentiviral vector encoding a CAR, an engineered TCR or a polypeptide that enhances the immune cell function, or a functional derivative thereof). Then about 24 hours after the stimulation initiation, the cells are washed and formulated for storage or administration. In other aspects, about 12 hours, about 22 hours, about 30 hours, about 40 hours, about 45 hours, about 50 hours, about 60 hours, or about 72 hours, after the stimulation initiation, the cells are washed and formulated for storage or administration.

D. Methods of Introducing Viral Vector into a Cell

Methods of introducing modifying agents (e.g., expression vectors, viral vectors, polynucleotides or nucleic acids) into a cell include physical, biological, chemical methods, and combination thereof. Expression vectors including a viral vector or expression vector of the present disclosure can be introduced into a host cell by any means known to persons skilled in the art. The expression vectors may include viral sequences for transfection, if desired. Alternatively, the expression vectors may be introduced by fusion, electroporation, biolistics (e.g., gene gun), transfection, lipofection (e.g., cationic liposome), polymer encapsulation, or the like. The host cell (e.g., immune cell) may be grown and expanded in culture before introduction of the expression vectors, followed by the appropriate treatment for introduction and integration of the vectors. The host cells (e.g., immune cells) may then be expanded and may be screened by virtue of a marker present in the vectors. Methods for producing cells including vectors and/or exogenous nucleic acids are well-known in the art. See, e.g., Sambrook et al., Molecular Cloning: A Laboratory Manual, Cold Spring Harbor Laboratory, New York (2001).

In some embodiments, the enriched apheresis product and/or the enriched target cell population can be modified using any method known in the art, such as activation, expansion, induction of apoptosis, genetic manipulation, induction of antigen-specificity. In some embodiments, the enriched apheresis product and/or enrichment of a target cell population can be modified by the addition of cytokines, cross-linking specific receptors, addition of antigen, introduction of nucleic acid molecules (DNA, RNA, and/or modified versions thereof), protein agents, addition of drugs or small molecules, or any combination thereof. In some embodiments, the introduction of modifying agents (e.g., expression vectors, viral vectors, exogenous nucleic acid molecules, polynucleotides or nucleic acids) comprises viral transfection (transduction), non-viral transfection, electroporation, lipofection, cationic liposome mediated transfection using lipofection, polymer encapsulation, peptide mediated transfection, or biolistic particle delivery systems such as “gene guns” (see, for example, Nishikawa et al., Hum Gene Ther 12(8):861-70 (2001).

1. Biological Methods

Biological methods for introducing a modifying agent of interest into a host cell (e.g. immune cell) include the use of expression vectors (e.g., viral vectors, exogenous nucleic acid molecules, polynucleotides or nucleic acids (DNA and RNA)). Viral vectors, and especially retroviral vectors (viral transfection), have become the most widely used method for inserting genes into mammalian (e.g., human cells). Viral vectors can be derived from lentivirus, poxviruses, herpes simplex virus I, adenoviruses and adeno-associated viruses, and the like. See for example, U.S. Pat. Nos. 5,350,674 and 5,585,362.

In some embodiments, a nucleic acid encoding a subject CAR, a subject engineered TCR, a subject KIR, a subject antigen-binding polypeptide, a subject cell surface receptor ligand, a subject tumor antigen, a subject switch receptor, subject dominant negative receptor, and/or a subject polypeptide that enhances immune function (e.g., T cell priming or T cell infiltration) can be introduced into a cell with an expression vector (viral transfection). Expression vectors (e.g., lentiviral vector or retroviral vector) comprising a nucleic acid encoding a subject CAR, a subject engineered TCR, a subject KIR, a subject antigen-binding polypeptide, a subject cell surface receptor ligand, a subject tumor antigen, a subject switch receptor, subject dominant negative receptor, and/or a subject polypeptide that enhances immune function (e.g., T cell priming or T cell infiltration) are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR, the subject engineered TCR, the subject KIR, the subject antigen-binding polypeptide, the subject cell surface receptor ligand, the subject tumor antigen, the subject switch receptor, subject dominant negative receptor, and/or a subject polypeptide that enhances immune function (e.g., T cell priming or T cell infiltration) in the host cell. In some embodiments, the adenovirus genome is a 36 kb, linear, double stranded DNA, where a foreign DNA sequence. For example, a nucleic acid encoding a subject CAR, a subject engineered TCR, a subject KIR, a subject antigen-binding polypeptide, a subject cell surface receptor ligand, a subject tumor antigen, a subject switch receptor, subject dominant negative receptor, and/or a subject polypeptide that enhances immune function (e.g., T cell priming or T cell infiltration) may be inserted to substitute large pieces of adenoviral DNA in order to make the expression vector of the present invention.

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR, a subject engineered TCR, a subject KIR, a subject antigen-binding polypeptide, a subject cell surface receptor ligand, a subject tumor antigen, a subject switch receptor, a subject dominant negative receptor, and/or a subject polypeptide that enhances immune function (e.g., T cell priming or T cell infiltration) into the viral genome at certain locations to produce a virus that is replication defective. Though the retrovirus vectors are able to infect a broad variety of cell types, integration and stable expression of the subject CAR, the subject engineered TCR, the subject KIR, the subject antigen-binding polypeptide, the subject cell surface receptor ligand, the subject tumor antigen, the subject switch receptor, subject dominant negative receptor, and/or a subject polypeptide that enhances immune function (e.g., T cell priming or T cell infiltration), requires the division of host cells.

Lentivirus vectors are derived from lentiviruses, which are complex retroviruses that, in addition to the common retroviral genes gag, pol, and env, contain other genes with regulatory or structural function. See, e.g., U.S. Pat. Nos. 6,013,516 and 5,994,136. Some examples of lentiviruses include the human immunodeficiency viruses (HTV-1, HTV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression of a nucleic acid encoding a subject CAR, a subject engineered TCR, a subject KIR, a subject antigen-binding polypeptide, a subject cell surface receptor ligand, a subject tumor antigen, a subject switch receptor, subject dominant negative receptor, and/or a subject polypeptide that enhances immune function (e.g., T cell priming or T cell infiltration). See, e.g., U.S. Pat. No. 5,994,136.

In some embodiments, the nucleic acids, encoding a subject CAR, a subject engineered TCR, a subject KIR, a subject antigen-binding polypeptide, a subject cell surface receptor ligand, a subject tumor antigen, a subject switch receptor, subject dominant negative receptor, and/or a subject polypeptide that enhances immune function (e.g., T cell priming or T cell infiltration), are introduced into the immune cell by viral transduction. In some embodiments, the viral transduction comprises contacting the immune cell with a viral vector comprising the one or more nucleic acids. In some embodiments, the viral vector is selected from the group consisting of a retroviral vector, sendai viral vectors, adenoviral vectors, adeno-associated virus vectors, and lentiviral vectors. Various markers that may be used are known in the art, and may include hprt, neomycin resistance, thymidine kinase, hygromycin resistance, etc.

The modified enriched apheresis product (e.g., immune cells) of the present invention (e.g., comprising a nucleic acid encoding a subject CAR, a subject engineered TCR, a subject KIR, a subject antigen-binding polypeptide, a subject cell surface receptor ligand, a subject tumor antigen, a subject switch receptor, subject dominant negative receptor, and/or a subject polypeptide that enhances immune function (e.g., T cell priming or T cell infiltration)) may be produced by stably transfecting host cells (e.g. immune cells) with an expression vector including a nucleic acid of the present disclosure.

Transfected cells (i.e. immune cells) expressing a nucleic acid encoding a CAR, a KIR, a TCR, a KIR, an antigen-binding polypeptide, a cell surface receptor ligand, a tumor antigen, a subject switch receptor, subject dominant negative receptor, and/or a subject polypeptide that enhances immune function (e.g., T cell priming or T cell infiltration) of the present disclosure may be expanded ex vivo. In some embodiments, transfected cells (i.e. immune cells) expressing a nucleic acid encoding a CAR, a KIR, a TCR, a KIR, an antigen-binding polypeptide, a cell surface receptor ligand, a tumor antigen, a subject switch receptor, subject dominant negative receptor, and/or a subject polypeptide that enhances immune function (e.g., T cell priming or T cell infiltration) of the present disclosure are not expanded ex vivo.

Additional methods for generating a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection).

2. Physical Methods

Physical methods for introducing a polynucleotide (RNA, or DNA) or an expression vector into a host cell include lipofection, particle bombardment, microinjection, electroporation, and the like. The expression vector or polynucleotide can be introduced into target cells using commercially available methods which include electroporation, such as 4D-Nucleofector™ Technology (Lonza Bioscience, Walkersville, MD), Amaxa Nucleofector-II (Amaxa Biosystems, Cologne, Germany)), ECM 830 (BTX) (Harvard Instruments, Boston, MA)), the Gene Pulser II (BioRad, Denver, CO), or Multiporator (Eppendorf, Hamburg Germany).

a. Electroporation

In some embodiments, the enriched apheresis product and/or enriched target cell population are transfected. In some embodiments, the enriched apheresis product and/or enriched target cell population are electroporated. In some embodiments, the cell transfection device comprises a flow electroporation chamber. For example, a chamber or system described in U.S. Pat. Nos. 5,720,921, 6,074,605; 7,141,425; 7,521,224, and 8,673,623, and U.S. Patent App. Pub. Nos. 2007/0128708A1, 2008/0182251A1, 2013/0196441, 2017/0233716A1, and Kim et al., Biosens Bioelectron 2008 23(9):1353-60

Electroporation applies an Electric field to cells to introduce pores (electropores), to the cell membrane through which (usually charged) macromolecules or agents can flow into the cell. Removal of the field permits the pores to re-seal, with the introduced molecules inside the cell. Important parameters for successful electroporation include the maximum voltage applied and the duration of the current pulse. The voltage and capacitance settings should also be optimized for each cell type, with the resistance of the electroporation buffer being important for choosing the initial instrument settings. Optimal stable and transient transformation occurs at about the same instrument settings, so transient expression can be used to optimize conditions when adapting to a new cell type.

Accordingly, electroporation-mediated administration into cells of nucleic acids including expression constructs presents a means for delivering an RNA of interest to a target cell. Electroporation-mediated administration can utilize any of the many available devices and electroporation systems known to those of skill in the art. Exemplary formulations and methodology of electroporation of nucleic acid constructs into mammalian cells are taught in US 2004/0014645, US 2005/0052630, US 2005/0070841, US 2004/0059285, US 2004/0092907, and US 2007/0128708. The various parameters including Electric field strength required for electroporation of any known cell type are generally known in the relevant research literature as well as numerous patents and applications in the field. See e.g., U.S. Pat. Nos. 6,678,556; 7,171,264, and 7,173 116.

In some embodiments, the cell transfection device is a commercially available apparatus for therapeutic application of electroporation selected from, but not limited to the MedPulser™ DNA Electroporation Therapy System (Inovio/Genetronics, San Diego, Calif), and are described in patents such as U.S. Pat. Nos. 6,567,694; 6,516,223; 5,993,434; 6, 181,964; 6,241,701, and 6,233,482.

In some embodiments, the cells are not activated prior to the transfection step. In some embodiments, prior to the transfection, the cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. 2006/0121005.

In some embodiments, the majority of unstimulated CAR T cells generated lentiviral electroporation (e.g., Nucleofection) can maintain a less differentiated phenotype when compared to stimulated CAR T cells population. For example, over at least about 30%, at least about 40%, at least about 45%, at least about 50%, at least about 55%, at least about 60%, at least about 65%, at least about 70% of Electric CAR T cells can be naïve CAR T cells. In some embodiments, electroporation of CAR lentiviral vector can result in effective CAR transgene integration into the T-cell genome. In some embodiments, the vector copy number per cell of Electric CAR T cells can be substantially similar to the copy number per cell of conventional (transduced) CAR T cells. In some embodiments, the electroporated CAR transgene can be expressed within about 0.5 hour, about 0.75 hour, about 1 hour, about 1.5 hours, about 2.0 hour, about 2.5 hour, about 3.0 hours, about 3.5 hours, about 3.5 hours, or at least about 5.0 hours post nucleofection.

In some embodiments, the enriched apheresis product and/or enriched target cell population can be modified using a suitable electroporation device, which can be for example from 4D-Nucleofector™ Technology (Lonza Bioscience, Walkersville, MD), Amaxa NUCLEOFECTOR™-II, Amaxa Biosystems (Cologne, Germany)), ECM 830 (BTX; Harvard Instruments, Boston, Mass.), the Gene Pulser II or the Gene Pulser MXCELL™ (BioRad, Denver, Colo.), Multiporator (Eppendort, Hamburg Germany), or FLOW ELECTROPORATION® technology (MaxCyte). In some embodiments, the transfection device is an ECM830 Electro Square Wave Porator (Harvard Apparatus BTX) and the cells are electroporated in a 2-mm cuvette (Harvard Apparatus BTX, Holliston, MA, USA).

A skilled artisan would understand that the pulse type, duration of pulsing, voltage, and frequency of use are dependent upon the type of instrument and the cell type; and that optimization of the efficiency of the transfection can be modulated based on the pulse type, duration of pulsing, voltage, frequency of use, and concentrations of the nucleic acid or particle being electroporated (e.g., DNA, RNA, expression vector, lentiviral vector, or lentiviral particle).

Electroporation can be done in one of two ways, batch electroporation or flow through electroporation.

i. Batch Electroporation

Electroporation has most often been performed in batch form, in relatively small volumes (around 1 ml, and often about 1×10⁶ cells) by placing a suspension of cells and macromolecules to be introduced into a cuvette including two electrodes which are connected to a pulse generator and arranged to deliver current through the suspension. In the batch format, one or more Electric field pulses are applied to the cells, and treated cells are generally transferred to medium to permit the cells to recover.

In one embodiment, a batch processing mode can be used in the methods and systems described herein. In this embodiment, cells are electroporated by shunting a suspension of washed enriched apheresis product or enriched target cell population into an electroporation chamber when a given concentration of target cells is reached (e.g., as detected by a detector) in the cell separation module, and adding a cell-modifying or cell-customizing agent such as an agent for generating CART cells (e.g., lentiviral vector, lentiviral particle, expression vector, DNA, RNA, or a protein) to the electroporation device chamber, and applying one or more Electric pulses from a pulse generator to the cell suspension. Electroporated cells can be re-introduced into the patient as each batch of cells is electroporated, or continuously if continuous electroporation is utilized.

ii. Flow Through Electroporation

In some embodiments, a flow-through or a continuous-flow electroporation system can be used. In this embodiment, the washed cells (e.g., the enriched apheresis product or enriched target cells) and the modifying agent (e.g., lentiviral particle, expression vector, lentiviral vectors, DNA, or RNA), are passed through an electroporation unit through which a voltage is constantly applied. In some embodiments, a flow-through or a continuous-flow electroporation system achieves treatment of up to 20 ml of cell suspension per minute, with a transfection efficiency as high as 75%. In some embodiments, a flow-through or a continuous-flow electroporation system achieves treatment of up to 1 to 10¹⁰ cells per second, 10⁴ to 10⁷ per second, 10⁵ to 10⁸ per second, or 10⁶ to 10⁹ per second, or batches of cells ranging from 1 cell to 10¹⁰ cells in a single transformation procedure with efficiency rates ranging up to about 70%, about 75%, about 80%, about 85%, about 90%, or about 95% or greater.

In some embodiments, the transfection device disclosed herein incorporate a fluidic channel with a constant depth, and/or variable width over its length, such that some portions are narrow and others are wider. The Electric field at any point is determined by the width of the channel, with narrower portions having a stronger field than wider ones. Widths and currents are selected such that the field only exceeds the transmembrane potential permitting electroporation at the narrow points, and alternating narrow and wider sections over the length of the channel provides an effect that approximates that of a pulsed field, without the need for a pulsed field generator. The flow rate through the channel, as well as the respective lengths of the wide and narrow sections, can be adjusted to adjust the time during which cells are exposed to current strong enough to electroporate them. Systems for continuous flow electroporation are described, for example in Wei & Li, Methods Mol. Biol. 1121: 99-110 (2014), Geng et al., J. Controlled Release 144: 91-100 (2010), U.S. Pat. Nos. 10,253,316; 6,617,154; 6,673,669; 7,029,916; 7,771,984; 9,546,350; or 10,253,316.

In some embodiments, a flow-through electroporation system, within the transfection device employs a fluidic system fabricated from polydimethylsiloxane (PDMS) on a glass substrate, incorporating channels of with alternating wide (10,000-5,000 μm, e.g., about 7,500 μm) and narrow (500-700 μm, e.g., about 500 μm) stretches. The inlet to the device can be connected to a conduit or tubing through which the cells are delivered from the buffer exchange device a to the transfection device. Wire electrodes inserted into the inlet and outlet of the flow-through electroporation system can be connected to a constant voltage power supply. Cells in suspension in electroporation buffer and including the lentiviral vector and/or nucleic acid encoding the CAR or TRC are pumped through the fluidic channel.

In some embodiments, the apheresis product is transfected with about 0.5 μl, about 1 μl, about 1.5 μl, about 2 μl, about 2.5 μl, about 3 μl, about 3.5 μl, about 4 μl, about 5 μl, about 6 μl, about 7 μl, about 8 μl, about 9 μl, about 10 μl, about 15 μl, or about 20 μl of the lentiviral vector. In some embodiments, the effective dose of the lentiviral vector comprises a multiplicity of infection (MOI) of about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.25, about 1.5, about 2.0, about 3.0, about 4.0, or about 5.0 at a MOI of about 0.01 to about 5.0. In some embodiments, the apheresis product is transfected with about 2 ul of the lentiviral vector at a MOI of about 0.08; 5 ul of the lentiviral vector at a MOI of about 0.2, or 10 ul of the lentiviral vector at a MOI of about 0.4.

In some embodiments, the electroporation module comprises a flow electroporation chamber. For example, a chamber or system described in U.S. Pat. Nos. 5,720,921; 6,074,605; and 7,141,425.

Another aspect of the present invention provides a novel way of electroporating cell.

Generally, during electroporation, a cell is contacted with an effective dose of the lentiviral vector prior to application of electricity to a cuvette containing the cell to be transfected. However, the present inventors found that electroporation can be toxic to the expression vector thereby reducing the transfection efficiency. Thus to enhance the efficiency of the electroporation, the present inventors electroporated the cells in the absence of expression vector and observed an enhancement in transfection efficiency. Accordingly, in some embodiments of the present disclosure, the cell may be contacted with the effective dose of the lentiviral vector before electroporation. In an alternative embodiment, the cell may be contacted with the effective dose of the lentiviral vector for up to about 4 hours after electroporation (e.g., application of electricity). For example, the cell may be contacted with the effective dose of the lentiviral vector for at least about 5-30 minutes, at least about 25-50 minutes; at least about 5-60 minutes, at least about 5-12 minutes, at least about 60-120 minutes, at least about 120-240 minutes after electroporation. Alternatively, the cell may be contacted with the effective dose of the lentiviral vector for at least about 1 minute, at least about 2 minutes, at least about 5 minutes, at least about 10 minutes, at least about 20 minutes, at least about 30 minutes, at least about 45 minutes, at least about 60 minutes, at least about 75 minutes, at least about 90 minutes, at least about 100 minutes, at least about 110 minutes, at least about 120 minutes, at least about 150 minutes, at least about 160 minutes, at least about 170 minutes, at least about 180 minutes, at least about 190 minutes, at least about 200 minutes, at least about 220 minutes, or at least about 240 minutes after electroporation.

Adding the lentiviral vector to the cell up to 4 hrs after electroporation (e.g., 1 minute to 2 hrs, or 1 minutes to 4 hrs) can reduce the amount of lentiviral particles that are killed by the electroporation, which can be toxic to the lentiviral particles. Accordingly, adding the lentiviral vector to the cell up to 4 hrs after electroporation can enhance CAR transfection. For example, addition of the lentiviral vector to the cell up to 4 hrs after electroporation can enhance CAR expression by about 10-15% when compared to a traditional electroporation process (e.g., adding the lentiviral to the cell before electroporation).

Thus in some embodiments of the manufacturing process disclosed herein, the transfecting the cell comprises electroporating the cell with a lentiviral vector and/or particle. In some embodiments, electroporating comprises adding the lentiviral vector to the cell prior to, simultaneously or after applying electricity to the cell. In some embodiments, electricity is applied to the cells after the addition of the lentiviral vector. In some embodiments of the manufacturing process disclosed herein, electricity is applied to the cells prior to the addition of the lentiviral vector. For example, electricity is applied to the cells for at least about 5-30 minutes, at least about 25-50 minutes; at least about 5-60 minutes, at least about 5-12 minutes, at least about 60-120 minutes, at least about 120-240 minutes prior to the addition of the lentiviral vector. Alternatively, electricity is applied to the cells for at least about 1 minute, at least about 2 minutes, at least about 5 minutes, at least about 10 minutes, at least about 20 minutes, at least about 30 minutes, at least about 45 minutes, at least about 60 minutes, at least about 75 minutes, at least about 90 minutes, at least about 100 minutes, at least about 110 minutes, at least about 120 minutes, at least about 150 minutes, at least about 160 minutes, at least about 170 minutes, at least about 180 minutes, at least about 190 minutes, at least about 200 minutes, at least about 220 minutes, or at least about 240 minutes prior to the addition of the lentiviral vector.

b. Cell Squeeze Microfluidic

In some embodiments, a Cell Squeeze Microfluidic is used to introduce an effective dose of a lentiviral vector into the enriched apheresis product. In some embodiments, the lentiviral vector is introduced into the mononuclear cells by forcing the cells under pressure through a constriction smaller in diameter. The rapid stretching, rapid compression, or pulse of high shear rate leads to uptake of molecules into the cytoplasm of the cell from the surrounding cell medium. This so-called “cell squeeze” microfluidic technology is applicable to a wide number of cell types, and well-suited for introducing materials to mononuclear cells. The cell squeeze microfluidic technology is described, for example, in WO 2013/059343 and US 2014/287509

3. Chemical Methods

Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, to confirm the presence of the nucleic acids in the host cell, a variety of assays may be performed. Such assays include, for example, molecular biological assays well known to those of skill in the art, such as Southern and Northern blotting, RT-PCR and PCR; biochemical assays, such as detecting the presence or absence of a particular peptide (e.g., immunological means (ELISAs and Western blots) or by assays described herein to identify agents falling within the scope of the invention.

Moreover, the nucleic acids may be introduced by any means, such as transducing the expanded host cells (e.g., immune cells; enriched apheresis product), transfecting the expanded host cells (e.g., immune cells; enriched apheresis product), and electroporating the expanded host cells (e.g., immune cells; enriched apheresis product). One nucleic acid may be introduced by one method and another nucleic acid may be introduced into the host cell (e.g., immune cells; enriched apheresis product) by a different method. In some embodiments, an expression system, such as a lentiviral or retroviral particle, may be introduced using viral transfection and chemical or physical transfection. For example, a lentiviral or retroviral particle may be transfected into a cell using electroporation.

4. Lentiviral Vector Transfection

In some embodiments, the methods described herein comprise transfecting the stimulated and/or unstimulated apheresis or blood product or enriched apheresis or blood product with one or more modifying agents. In some embodiments, the one or more modifying agents are selected from the group consisting of a small molecule agent, a biologic agent, a therapeutic, a protein, a peptide, a protein therapeutic, a peptide therapeutic, a nucleic acid, DNA, RNA, mRNA, a chimeric antigen receptor, a heterologous T cell receptor, an expression vector, a viral vector, a vector, a retroviral vector, a lentiviral vector, an adenoviral vector, and an adeno-associated viral vector. The modifying agent may include viruses that are not permissive to human or eukaryotic cells (e.g., viruses that cannot naturally infect or enter human or eukaryotic cells). In some embodiments, the modifying agent can also be selected from a retroviral vector, or a lentiviral vector. In some embodiments, the modifying agent can be a retroviral vector, a lentiviral vector, an adenoviral vector, or an adeno-associated viral vector. In one embodiment, the modifying agent is a lentiviral vector or a retroviral vector. In some embodiments, the lentiviral vector is a lentiviral particle.

In some embodiments, the transfection is a viral transfection (e.g., a viral transduction) or the transfection is an electroporation of a nucleic acid encoding, or an electroporation of a lentiviral vector comprising a nucleic acid sequence encoding a chimeric antigen receptor (CAR), an engineered T cell receptor (TCR), and/or a polypeptide that enhances the immune cell function, or a functional derivative thereof.

In some embodiments, the transfection is selected from the group consisting of viral transfection (e.g., viral transduction), non-viral transfection, and/or hybrid of viral- and non-viral transfection. In some embodiments, transfection is selected from the group consisting of electroporation, laser beam, gene injection, spinoculation, sonoporation, magentofection, metal-coated nanoparticles, magnetic-conjugated adeno-associated virus, micro/nanoparticle-mediated transfection, lipofection, lipid-based transfection, anionic liposome, cationic liposome-mediated transfection, cationic polymer, polymer encapsulation, peptide mediated transfection, calcium phosphate, dendrimers, flowfection, photoporation, soluporation, transient cell-membrane disruption, deformation, squeezing, stretching, pinching, weakening, elongation, thinning, biolistic particle delivery systems and a combination thereof.

In some embodiments of the aforementioned methods, the cells are transduced by spinoculation. For example, transducing the apheretic cell product with a viral vector comprises subjecting the apheretic cell product and viral vector to a centrifugal force to enhance the update of the viral particle by the cell, thereby enhancing the transduction efficiency.

In some embodiments, the apheresis product (e.g., the population of immune cells, the population of CD4⁺ and CD8⁺ cells, or the population eukaryotic donor cells) is transfected by electroporation of a viral particle (i.e., a hybrid viral and non-viral transfection). In some embodiments, the apheresis product (e.g., the population of immune cells, the population of CD4⁺ and CD8⁺ cells, or the population eukaryotic donor cells) is transfected by electroporation and/or viral transfection (transduction). In some embodiments, the apheresis product (e.g., the population of immune cells, the population of CD4⁺ and CD8⁺ cells, or the population eukaryotic donor cells) is transfected by viral transfection and/or lipid-based transfection. In some embodiments, the apheresis product (e.g., the population of immune cells, the population of CD4⁺ and CD8⁺ cells, or the population eukaryotic donor cells) is transfected by viral transfection and liposome based transfection.

In some embodiments, transfecting the population of apheresis product (e.g., the population of immune cells, the population of CD4⁺ and CD8⁺ cells, or the population eukaryotic donor cells) with one or more modifying agents (e.g., a nucleic acid sequence encoding a chimeric antigen receptor (CAR), an engineered T cell receptor (TCR), and/or a polypeptide that enhances the immune cell function, or a functional derivative thereof) occurs simultaneously with the stimulation and/or activation of the population of apheresis product with the one or more stimulating agents (e.g., cytokines, recombinant costimulatory molecules, anti-CD3 antibodies or fragments thereof, anti-CD28 antibodies or fragments, small drug inhibitors, and/or a combination thereof) as described above.

In some embodiments, transfecting the population of apheresis product (e.g., the population of immune cells, the population of CD4⁺ and CD8⁺ cells, or the population eukaryotic donor cells) with one or more modifying agents (e.g., a nucleic acid sequence encoding a chimeric antigen receptor (CAR), an engineered T cell receptor (TCR), and/or a polypeptide that enhances the immune cell function, or a functional derivative thereof) occurs no later than 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours or 10 hours after the beginning of the stimulation and/or activation of the apheresis product with the one or more stimulating agent as described above.

In some embodiments, transfecting the population of apheresis product (e.g., the population of immune cells, the population of CD4⁺ and CD8⁺ cells, or the population eukaryotic donor cells) with one or more modifying agents (e.g., a nucleic acid sequence encoding a chimeric antigen receptor (CAR), an engineered T cell receptor (TCR), and/or a polypeptide that enhances the immune cell function, or a functional derivative thereof) occurs no later than 5 hours after the beginning of the stimulation and/or activation of the apheresis product with the one or more stimulating agent described above.

In some embodiments, transfecting the population of apheresis product (e.g., the population of immune cells, the population of CD4⁺ and CD8⁺ cells, or the population eukaryotic donor cells) with one or more modifying agents (e.g., a nucleic acid sequence encoding a chimeric antigen receptor (CAR), an engineered T cell receptor (TCR), and/or a polypeptide that enhances the immune cell function, or a functional derivative thereof) occurs no later than 4 hours after the beginning of the stimulation and/or activation of the apheresis product with the one or more stimulating agent described above.

In some embodiments, transfecting the population of apheresis product (e.g., the population of immune cells, the population of CD4⁺ and CD8⁺ cells, or the population eukaryotic donor cells) with one or more modifying agents (e.g., a nucleic acid sequence encoding a chimeric antigen receptor (CAR), an engineered T cell receptor (TCR), and/or a polypeptide that enhances the immune cell function, or a functional derivative thereof) occurs no later than 3 hours after the beginning of the stimulation and/or activation of the apheresis product with the one or more stimulating agent described above.

In some embodiments, transfecting the population of apheresis product (e.g., the population of immune cells, the population of CD4⁺ and CD8⁺ cells, or the population eukaryotic donor cells) with one or more modifying agents (e.g., a nucleic acid sequence encoding a chimeric antigen receptor (CAR), an engineered T cell receptor (TCR), and/or a polypeptide that enhances the immune cell function, or a functional derivative thereof) occurs no later than 2 hours after the beginning of the stimulation and/or activation of the apheresis product with the one or more stimulating agent described above.

In some embodiments, transfecting the population of apheresis product (e.g., the population of immune cells, the population of CD4⁺ and CD8⁺ cells, or the population eukaryotic donor cells) with one or more modifying agents (e.g., a nucleic acid sequence encoding a chimeric antigen receptor (CAR), an engineered T cell receptor (TCR), and/or a polypeptide that enhances the immune cell function, or a functional derivative thereof) occurs no later than 1 hour after the beginning of the stimulation and/or activation of the apheresis product with the one or more stimulating agent described above.

In some embodiments, transfecting the population of apheresis product (e.g., the population of immune cells, the population of CD4⁺ and CD8⁺ cells, or the population eukaryotic donor cells) with one or more modifying agents (e.g., a nucleic acid sequence encoding a chimeric antigen receptor (CAR), an engineered T cell receptor (TCR), and/or a polypeptide that enhances the immune cell function, or a functional derivative thereof) occurs without the stimulation and/or activation of the apheresis product with the one or more stimulating agent described above.

In some embodiments, the transfected apheresis product (e.g., the population of immune cells or the population of eukaryotic donor cells) is selected from the group consisting of mononuclear cells, Lymphocytes rich cells, B lymphocytes, T lymphocytes, CD4⁺ T lymphocytes, CD8⁺ T lymphocytes, dendritic cells, monocytes, natural killer (NK) cells, natural killer T (NKT) cells, T-regulatory cells, CD4⁺ T-helper cells, CD8⁺ cytotoxic T lymphocytes (CTLs), CD62L⁺ cells, CD27⁺ cells, CCR7⁺ cells, CD45RO⁻ cells, CD45RA±cells, neutrophils, basophils, eosinophils, megakaryocytes, stem cells, hematopoietic stem cells (HSCs), hematopoietic progenitor cells (HPCs), CD34⁺ cells, CD34⁺ peripheral blood stem cells, lymphokine-activated killer cells (LAKs), tumor infiltrating lymphocytes (TILs), mesenchymal stem cells, mast cells, a monocyte, a macrophage, a neutrophil, a basophil, an eosinophil, a dendritic cell, a megakaryocyte, and combinations thereof.

In some embodiments, the concentration of the transfected apheresis product (e.g., the population of immune cells, the population of CD4⁺ and CD8⁺ cells, or the population eukaryotic donor cells) can be at least about 0.7×10⁷, at least about 0.8×10⁷, at least about 0.9×10⁷, at least about 1×10⁷, at least about 2×10⁷, at least about 4×10⁷, at least about 6×10⁷, at least about 8×10⁷, at least about 1×10⁸, or at least about 5×10⁸ cells/mL. In some embodiments, the concentration of the apheresis product (e.g., the population of immune cells, the population of CD4⁺ and CD8⁺ cells, or the population eukaryotic donor cells) can be from about 0.5×10⁶ cells/mL to about 4×10⁶ cells/mL. The concentration of the apheresis product (e.g., the population of immune cells, the population of CD4⁺ and CD8⁺ cells, or the population eukaryotic donor cells) can also be from about 0.5×106 cells/mL to about 1×10⁸ cells/mL. In some embodiments, the concentration of the apheresis product can also be from about 4.0×10⁶ cells/mL to about 1×10⁸ cells/mL.

In some embodiments of the methods disclosed herein, the methods further comprise adding an adjuvant or a transfection enhancement reagent in the cell culture medium to enhance the transfection (e.g., transduction) efficiency. In some embodiments, the adjuvant or transduction enhancement reagent comprises a cationic polymer. In some embodiments, the adjuvant or transduction enhancement reagent is chosen from: LentiBOOST™ (Sirion Biotech), vectofusin-1, F108 (Poloxamer 338 or Pluronic® F-38), protamine sulfate, hexadimethrine bromide (Polybrene), PEA, Pluronic F68, Pluronic F127, Synperonic or LentiTrans™. In some embodiments, the transduction enhancement reagent is LentiBOOST™ (Sirion Biotech). In some embodiments, the transduction enhancement reagent is F108 (Poloxamer 338 or Pluronic® F-38).

The manufacturing process disclosed herein (e.g., Electric CAR T cell) is made possible by a new strategy of transducing immune cells with a lentiviral vector (comprising a nucleic acid encoding a CAR, a TCR and/or a polypeptide that enhances the immune cell function, or a functional derivative thereof.

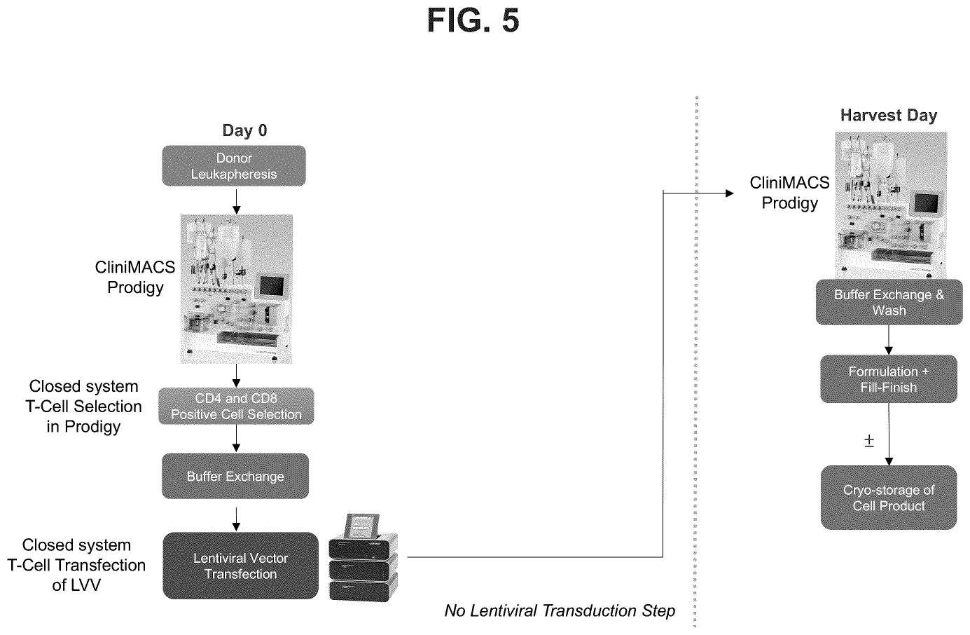

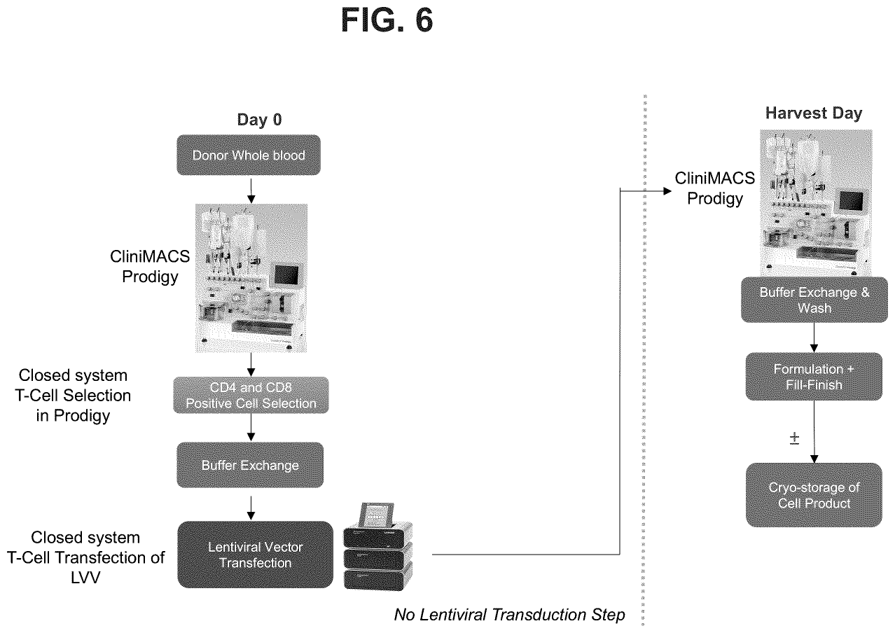

The CAR T cell manufacturing method relies on a hybrid transfection method that combines a biological transfection (virus based transducing) as described in Example 1 below and a physical transfection, such as electroporation. Specifically, lentiviral particles are electroporated into immune cells or T cells. The electroporation of lentiviral particles into cells speed up the viral transfection/transduction and permits the 1-day manufacturing of CAR T cells (e.g., Electric CAR T cells) without post transfection culture and/or expansion. As shown in FIGS. 5-8, and 10, electroporated CAR T cells can be harvested within hours. As further discussed in Examples 3 and 4, and shown in FIG. 11 and Tables 4-6, such CAR T cells (e.g., Electric CAR T cells) efficiently killed target cells.

Accordingly, in some embodiments of the manufacturing process disclosed herein, the apheresis product (e.g., the population of immune cells, the population of CD4⁺ and CD8⁺ cells, or the population of eukaryotic donor cells) is transfected with an effective dose of a lentiviral vector or retroviral vector. The lentiviral vector or retroviral vector may comprise a nucleic acid sequence encoding a chimeric antigen receptor (CAR), an engineered T cell receptor (TCR), and/or a polypeptide that enhances the immune cell function, or a functional derivative thereof.

In some embodiments, the manufacturing process contemplated herein comprises transfecting the enriched apheresis product with an effective dose of a lentiviral vector or retroviral vector that comprises a multiplicity of infection (MOI) of about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.25, about 1.5, about 2.0, about 3.0, about 4.0, or about 5. In some embodiments, the manufacturing process contemplated herein comprises transfecting the enriched apheresis product with an effective dose of a lentiviral vector or retroviral vector at an MOI of about 10 or 20. In a preferred embodiment, the manufacturing process contemplated herein comprises transfecting the enriched apheresis product with an effective dose of a lentiviral vector or retroviral vector at an MOI of about 0.08, 0.2, or 0.4.

In some embodiments, the manufacturing process contemplated herein comprises transfecting the enriched apheresis product with about 0.5, about 1, about 1.5, about 2, about 2.5, about 3, about 3.5, about 4, about 5, about 6, about 7, about 8, about 9, about 10, about 15, or about 20 μl of the lentiviral vector at a multiplicity of infection (MOI) of 0.01 to about 20.0.

In some embodiments, the manufacturing process contemplated herein comprises transfecting the enriched apheresis product with 2 μl of the lentiviral vector or retroviral vector at a MOI of about 0.08; 5 μl of the lentiviral vector or retroviral vector at a MOI of about 0.2, or 10 μl of the lentiviral vector or retroviral vector at a MOI of about 0.4.

The lentiviral vector may be based on a virus selected from the group consisting of a retrovirus, an alpha retrovirus, a beta retrovirus, a gamma retrovirus, a delta retrovirus, and an epsilon retrovirus. For example, the lentiviral vector may be based on a Human immunodeficiency virus (HIV), an Equine infectious anaemia virus (EIAV), a visna-maedi virus (VMV) virus, a caprine arthritis-encephalitis virus (CAEV), a feline immunodeficiency virus (Hy), a bovine immune deficiency virus (BIV), a VISNA virus, and a simian immunodeficiency virus (SIV). In some embodiments, the lentiviral vector may be pseudotyped with an envelope glycoprotein (Env) from a virus selected from the group consisting of a murine leukemia virus (MLV), a vesicular stomatitis virus (VSV) Indiana strain, VSV New Jersey strain, Cocal virus, Chandipura virus, Piry virus, spring viremia of carp virus (SVCV), Sigma virus, infectious hematopoietic necrosis virus (IHNV), Mokola virus, rabies virus CVS virus, Isfahan virus, Alagoas virus, Calchaqui virus, Jurona vrus, La Joya virus, Maraba virus, Feline Endogenous Retrovirus (RD114) Envelope Protein, Perinet virus, Yug Bugdanovac virus, a prototypic foamy virus (PFV), and gibbon ape leukemia virus (GaLV). In some embodiments, the lentiviral vector may be pseudotyped with an envelope glycoprotein (Env) selected from the group consisting of vesicular stomatitis virus (VSV) Indiana strain, VSV New Jersey strain, and Cocal virus.

. In some embodiments, the manufacturing process contemplated herein comprises transfecting the enriched apheresis product with 2 μl of the lentiviral vector or retroviral vector at a MOI of about 0.08; 5 μl of the lentiviral vector or retroviral vector at a MOI of about 0.2, or 10 μl of the lentiviral vector or retroviral vector at a MOI of about 0.4.

In some embodiment the lentiviral vector comprises a heterologous viral envelope protein (Env) selected from the group consisting of a VSV-G of the Indiana strain, VSV-G of the New Jersey strain, the Cocal virus envelope protein, the Isfahan virus envelope protein, Chandipura virus envelope protein, Pyri virus envelope protein, a murine leukemia virus (MLV) envelope glycoprotein, a SVCV virus envelope protein, and a variant thereof.

In some embodiments, the lentiviral vector comprises a nucleotide sequence encoding the VSV-G envelope protein, or a VSV G protein variant.

In some embodiments of the manufacturing process disclosed herein, the apheresis product (e.g., the population of immune cells, the population of CD4⁺ and CD8⁺ cells, or the population of eukaryotic donor cells) is transfected with an effective dose of a lentiviral vector or retroviral vector comprising a VSV G envelope protein.

E. Post-Transfection Ex Vivo Culture: Activation and Stimulation

In another aspect, the methods disclosed herein further comprise the step of stimulating and activating the population of transfected cells (e.g., the population modified lymphocytes, the population of modified immune cells, the population of modified CD4⁺ and CD8⁺ cells, or the population of modified eukaryotic donor cells) with one or more stimulating agents to produce a population of activated cells (e.g. a population of activated modified immune cells, a population of activated modified CD4⁺ and CD8⁺ cells, or a population of activated modified eukaryotic cells).

In yet another aspect, the methods disclosed herein further comprise the step of culturing and/or expanding the population of activated modified immune cells, the population of activated modified mononuclear cells, the population of activated modified CD4⁺ and CD8⁺ cells, or the population of activated modified eukaryotic donor cells for a predetermined time to produce a population of engineered cells or a population of engineered CD4⁺ and CD8⁺ cells.

In some embodiments, the expanding step is performed under shaking conditions or rotating conditions. In some embodiments, the expanding step is performed in a closed system. In some embodiments, the expanding step is performed using a serum-free culture medium and/or in the presence of one or more stimulating agents described herein In some embodiments, the expanding step is performed in the presence of one or more stimulating agents described herein.

In some embodiments, the population of activated apheresis product (e.g., the population of activated modified immune cells, the population of activated modified CD4⁺ and CD8⁺ cells, and/or the population of activated modified eukaryotic donor cells) is expanded for at least about 2-fold, at least about 3-fold, at least about 4-fold, at least about 5-fold, at least about 6-fold, at least about 7-fold, at least about 8 fold, at least about 10-fold, at least about 15-fold, at least about 20-fold, or at least about 25-fold compared to the population of cells before transfection or immediately after transfection.

In some embodiments, the population of cells is expanded by no more than about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, or about 60%, for example, as assessed by the number of living cells, compared to the population of cells before transfection or immediately after transfection. In some embodiments, the population of cells is expanded by no more than about 5%, no more than about 10%, no more than about 15%, no more than about 20%, no more than about 25%, no more than about 30%, no more than about 35%, or no more than about 40%, for example, as assessed by the number of living cells, compared to the population of cells before transfection or immediately after transfection.

In some embodiments, the population of cells is expanded by no more than about 1, about 1.5, about 2, about 2.5, about 3, about 3.5, about 4, about 4.5, about 5, about 6, about 7, about 8, about 9, about 10, about 11, about 12, about 16, about 20, about 24, about 36, about 48, about 55, about 60, about 65, about 70, about 72, about 80, about 90, or about 96 hours, as assessed by the number of living cells.

In some embodiments, during the culture and expansion step, the population of transfected cells is contacted in vitro with an agent that stimulates a CD3/TCR complex (e.g., an anti-CD3 antibody) and/or an agent that stimulates a costimulatory molecule (e.g., an anti-CD28 antibody), and/or growth factor receptor on the surface of the cells. In some embodiments, the population of transfected cells is stimulated for the entire expansion period. In some embodiments, the population of transfected cells is stimulated for at least about 20 hours, at least about 21 hours, at least about 22 hours, at least about 23 hours, at least about 24 hours, at least about 25 hours, at least about 26 hours, at least about 27 hours, or at least about 28 hours. In some embodiments, the population of transfected cells is cultured and expanded in media comprising no more than about 0%, about 0.5%, about 1%, about 1.5%, 2%, about 2.5%, about 3%, about 3.5%, about 4%, about 4.5%, about 5%, about 5.5%, about 6%, about 6.5%, about 7%, 7.5%, or 8% serum. In some embodiments, the cytokine process provided herein is conducted in cell media comprising a LSD1 inhibitor, a MALT1 inhibitor, or a combination thereof.

In some embodiments, the population of cells (i.e., the apheresis product) manufactured the methods disclosed herein shows a higher percentage of naive immune cells among the CAR-expressing cells. For example, the percentage of naive immune cells among the CAR-expressing cells can be at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 25%, at least about 30%, at least about 35%, at least about 40%, at least about 45%, at least about 50%, at least about 55%, or at least about 60% higher, as compared with cells made by an otherwise conventional method of manufacturing CAR T cells.

In some embodiments of the methods disclosed herein, the population of transfected (e.g., the population modified lymphocytes, the population of modified immune cells, the population of modified CD4⁺ and CD8⁺ cells, or the population of modified eukaryotic donor cells) is not further cultured following transfection. In some embodiments, the population of transfected cells (e.g., the population of modified immune cells, the population of modified CD4⁺ and CD8⁺ cells, or the population of modified eukaryotic donor cells) is not activated with one or more stimulating agents following transfection. In that embodiment, the population of transfected cells (e.g., the population of modified immune cells, the population of modified CD4⁺ and CD8⁺ cells, or the population of modified eukaryotic donor cells) is also not expanded ex vivo following transfection. In that embodiment, the population of transfected cells is harvested within 24 hours of transfection. In this embodiment, prior to the transfecting step (c), the population of enriched cells (e.g., the population of immune cells, the population of CD4⁺ and CD8⁺ cells, or the population of eukaryotic donor cells) may be stimulated and/or activated with one or more stimulating agents prior to transfection.

F. Harvesting

In another aspect, the methods disclosed herein further comprise the step of harvesting the population of modified apheresis product (e.g., the population of modified lymphocytes, the population of modified immune cells, the population of modified CD4⁺ and CD8⁺ cells, or the population of modified eukaryotic donor cells) for cryopreservation or administration.

In some embodiments, the harvesting comprises selecting and enriching for engineered lymphocytes, engineered immune cells, engineered CD4⁺ and CD8⁺ cells, or engineered donor eukaryotic cells. In some embodiments, harvesting further comprises formulating the engineered lymphocytes, the engineered immune cells, the engineered CD4⁺ and CD8⁺ cells, or the engineered donor eukaryotic cells for cryopreservation or administration to a subject in need thereof.

In some embodiments, when the transfected apheresis product (e.g., the population of modified lymphocytes, the population of modified immune cells, the population of modified CD4⁺ and CD8⁺ cells, or the population of modified eukaryotic donor cells) is further cultured and expanded ex vivo, the transfected apheresis product (e.g., the population of modified lymphocytes, the population of modified immune cells, the population of modified CD4⁺ and CD8⁺ cells, or the population of modified eukaryotic donor cells) can be cultured for a predetermined time before harvesting the engineered population of desired cells (e.g., a population of engineered lymphocytes, a population of engineered immune cells, a population of engineered CD4⁺ and CD8⁺ cells, or a population of engineered eukaryotic donor cells).

In some embodiments, the expansion predetermined time can be less than or equal to about 24 hours; less than or equal to about 30 hours; less than or equal to about 48 hours; less than or equal to about 72 hours; less than or equal to about 96 hours; or less than or equal to about 120 hours. In some embodiments, the expansion predetermined time can be less than about 0.5 hour, less than about 1 hour, less than about 2 hours, less than about 3 hours, less than about 4 hours, less than about 5 hours, less than about 6 hours, less than about 7 hours, less than about 8 hours, less than about 9 hours, less than about 10 hours, less than about 11 hours, less than about 12 hours, less than about 13 hours, less than about 14 hours, less than about 15 hours, less than about 16 hours, less than about 17 hours, less than about 18 hours, less than about 19 hours, less than about 20 hours, less than about 21 hours, less than about 22 hours, or less than about 23 hours.

In some embodiments, the expansion predetermined time can be about 1 day, about 2 days, about 3 days, about 4 days, about 5 days, about 6 days, about 7 days, about 8 days, about 9 days, about 10 days, about 11 days, about 12 days, about 13 days, about 14 days, or more days.

In some embodiment of the method for manufacturing a population of engineered immune cells disclosed herein, the time from enriching and/or obtaining the apheresis product (e.g., population of immune cells, the population of CD4⁺ and CD8⁺ cells, or the population of eukaryotic donor cells) to harvesting the engineered cells (e.g., the engineered lymphocytes, engineered immune cells, the engineered CD4⁺ and CD8⁺ cells, or the engineered eukaryotic donor cells) can be about 12 hours or less, about 18 hours or less, about 20 hours or less, about 22 hours or less, about 24 hours or less, about 26 hours or less, about 28 hours or less, about 30 hours or less, about 32 hours or less, about 36 hours or less, about 40 hours or less, about 45 hours or less, about 48 hours or less, about 50 hours or less, about 55 hours or less, about 60 hours or less, about 65 hours or less, about 70 hours or less, or about 72 hours or less.

In some embodiments, the time from enriching and/or obtaining the apheresis product (e.g., population of immune cells, the population of CD4⁺ and CD8⁺ cells, or the population of eukaryotic donor cells) to harvesting the engineered cells (e.g., the engineered lymphocytes, engineered immune cells, the engineered CD4⁺ and CD8⁺ cells, or the engineered eukaryotic donor cells) from about 18 hours to about 72 hours, from about 18 hours to about 36 hours, from about 18 hours to about 24 hours, from about 24 hours to about 72 hours, from about 24 hours to about 36 hours, or from about 36 hours to about 72 hours.

In some embodiments, the time from enriching and/or obtaining the apheresis product (e.g., population of immune cells, the population of CD4⁺ and CD8⁺ cells, or the population of eukaryotic donor cells) to harvesting the engineered cells (e.g., the engineered lymphocytes, engineered immune cells, the engineered CD4⁺ and CD8⁺ cells, or the engineered eukaryotic donor cells) can be less than about 2 hours, less than about 3 hours, less than about 4 hours, less than about 5 hours, less than about 6 hours, less than about 7 hours, less than about 8 hours, less than about 9 hours, less than about 10 hours, less than about 11 hours, less than about 12 hours, less than about 13 hours, less than about 14 hours, less than about 15 hours, less than about 16 hours, less than about 17 hours, less than about 18 hours, less than about 19 hours, less than about 20 hours, less than about 21 hours, less than about 22 hours, less than about 23 hours, less than about 24 hours, less than about 30 hours, less than about 35 hours, less than about 40 hours, less than about 45 hours, less than about 50 hours, less than about 55 hours, less than about 60 hours, less than about 65 hours, less than about 70 hours, or less than about 72 hours.

In some embodiments, the time from enriching and/or obtaining the apheresis product (e.g., population of immune cells, the population of CD4⁺ and CD8⁺ cells, or the population of eukaryotic donor cells) to harvesting the engineered cells (e.g., the engineered lymphocytes, engineered immune cells, the engineered CD4⁺ and CD8⁺ cells, or the engineered eukaryotic donor cells) about 1 day, about 2 days, about 3 days, about 4 days, about 5 days, about 6 days, about 7 days, about 8 days, about 9 days, about 10 days, about 11 days, about 12 days, about 13 days, about 14 days, or more days. In some embodiments, the time from enriching and/or obtaining the apheresis product (e.g., population of immune cells, the population of CD4⁺ and CD8⁺ cells, or the population of eukaryotic donor cells) to harvesting the engineered cells (e.g., the engineered lymphocytes, engineered immune cells, the engineered CD4⁺ and CD8⁺ cells, or the engineered eukaryotic donor cells) can be about 1 day, about 3 days, about 4 days, about 5 days, or about 6 days.

In some embodiments, the electroporating step, the activating step and/or the expanding step are performed in a closed system, a semi-closed, and/or a functionally closed system. The manufacturing process disclosed herein can occur in a closed system where the likelihood of contamination is minimal because limited manual manipulation. Thus, the closed system can minimize the risk of contamination (e.g., environmental contamination). In some embodiments, T cell separation, activation, transduction, incubation, and washing are all performed in a closed system. In some embodiments of the methods disclosed herein, the methods are performed in separate devices. In some embodiments, T cell separation, activation and transduction, incubation, and washing are performed in separate devices. In some embodiments, the closed system is selected from the group consisting of a closed bag system, an automated closed cell sample processing system, and a bioreactor (e.g., such as Xuri™ Cell Expansion System W25—Girgin Ltd (or any GE Healthcare's WAVE Bioreactor™ technology).

In certain embodiments, the closed system is a closed bag culture system, using any suitable cell culture bags (e.g., Mitenyi Biotec MACS® GMP Cell Differentiation Bags, Origen Biomedical PermaLife™ Cell Culture bags, or Origen PermaLife™ PL240 bag). In some embodiments, the cell culture bags used in the closed bag culture system are coated with a recombinant human fibronectin protein during the transduction step. In certain embodiments, the cell culture bags used in the closed bag culture system are coated with a recombinant human fibronectin protein fragment during the transduction step. The recombinant human fibronectin fragment may include three functional domains: a central cell-binding domain, heparin-binding domain II, and a CS1-sequence. The recombinant human fibronectin protein or fragment thereof may be used to increase gene efficiency of retroviral transduction of immune cells by aiding co-localization of target cells and viral vector. In certain embodiments, the recombinant human fibronectin fragment is RetroNectin® (Takara Bio, Japan). In certain embodiments, the cell culture bags may be coated with recombinant human fibronectin fragment at a concentration of about 1-60 μg/mL, preferably 1-40 μg/mL. In certain embodiments, the cell culture bags may be coated with recombinant human fibronectin fragment at a concentration of about 1-20 μg/mL, 20-40 μg/mL, or 40-60 μg/mL.

In some embodiments of the methods disclosed herein, the enriched apheresis product (e.g., T cells) is stimulated and/or activated, and transfected in a cell culture flask comprising a gas-permeable membrane at the base that supports large media volumes without substantially compromising gas exchange. In some embodiments, cell growth is achieved by providing access, substantially uninterrupted access, to nutrients through convection.

### III. Lentiviral Vectors

One aspect of the present disclosure provides a lentiviral vector described herein.

Another aspect of the present disclosure provides a lentiviral vector comprising a polynucleotide sequence encoding at least one heterologous viral envelope protein originating from a virus; a polynucleotide sequence encoding at least one viral rev protein; a polynucleotide sequence encoding at least one viral gag protein and at least one viral pol protein; and/or a polynucleotide sequence encoding a chimeric antigen receptor or an engineered T cell receptor (TCR).

The lentiviral vector may be based on a virus selected from the group consisting of a retrovirus, an alpha retrovirus, a beta retrovirus, a gamma retrovirus, a delta retrovirus, and an epsilon retrovirus. For example, the lentiviral vector may be based on a Human immunodeficiency virus (HIV), an Equine infectious anaemia virus (EIAV), a visna-maedi virus (VMV) virus, a caprine arthritis-encephalitis virus (CAEV), a feline immunodeficiency virus (Hy), a bovine immune deficiency virus (BIV), a VISNA virus, and a simian immunodeficiency virus (SIV). In some embodiments, the lentiviral vector may be pseudotyped with an envelope glycoprotein (Env) from a virus selected from the group consisting of a murine leukemia virus (MLV), a vesicular stomatitis virus (VSV) Indiana strain, VSV New Jersey strain, Cocal virus, Chandipura virus, Piry virus, spring viremia of carp virus (SVCV), Sigma virus, infectious hematopoietic necrosis virus (IHNV), Mokola virus, rabies virus CVS virus, Isfahan virus, Alagoas virus, Calchaqui virus, Jurona vrus, La Joya virus, Maraba virus, Feline Endogenous Retrovirus (RD114) Envelope Protein, Perinet virus, Yug Bugdanovac virus, a prototypic foamy virus (PFV), and gibbon ape leukemia virus (GaLV). In some embodiments, the lentiviral vector may be pseudotyped with an envelope glycoprotein (Env) selected from the group consisting of vesicular stomatitis virus (VSV) Indiana strain, VSV New Jersey strain, and Cocal virus.

In some embodiments of the lentiviral vector described herein, the viral envelope protein (Env) comprises a VSV-G glycoprotein selected from the group consisting of VSV-G of the Indiana strain, VSV-G of the New Jersey strain, the Cocal virus envelope protein, the Isfahan virus envelope protein, Chandipura virus envelope protein, Pyri virus envelope protein, a murine leukemia virus (MLV) envelope glycoprotein, a SVCV virus envelope protein, and a variant thereof. The lentiviral vector may also comprise a nucleotide sequence encoding a heterologous VSV-G envelope protein.

The heterologous VSV G envelope protein may be codon-optimized for human expression. Alternatively, the heterologous VSV G envelope protein may be a VSV G protein variant.

In some embodiments, the lentiviral vector comprises a nucleotide sequence encoding the VSV-G envelope protein, or a VSV G protein variant.

In some embodiments of the lentiviral vector described herein, the heterologous envelope protein may be under the control of a transcriptional regulatory element. The transcriptional regulatory element maybe a promoter selected from a eukaryotic promoter or a constitutive promoter.

The lentiviral vector described herein can further comprise a transcriptional regulatory element and the transcriptional regulatory element may be upstream of the heterologous envelope glycoprotein (i.e. in the 5′ direction of the nucleotide sequence encoding the heterologous envelope glycoprotein). For example, the transcriptional regulatory element may control the expression (i.e. transcription and, accordingly, but optionally, translation) of the nucleic acid encoding the heterologous envelope glycoprotein. In some embodiments, the transcriptional regulatory element is constitutively active or is a constitutive promoter. In exemplary embodiments, the constitutively active transcriptional regulatory element or the constitutive promoter may be a cytomegalovirus (CMV) promoter, such as the CMV major immediate early promoter (CMV IE1), a murine stem cell virus promoter, Elongation Factor-1 alpha promoter (EF-1 alpha), a viral simian virus 40 (SV40) (e.g., early or late), a Moloney murine leukemia virus (MoMLV), an ubiquitin C promoter, a phosphoglycerokinase (PGK) promoter, a Rous sarcoma virus (RSV), or herpes simplex virus (HSV) (thymidine kinase) promoter.

In other embodiments, the activity of the transcriptional regulatory element may be inducible or the promoter may be an inducible promoter. In some embodiments, the transcriptional regulatory element may be a eukaryotic promoter, such as phosphoglycerate kinase promoter. Other transcriptional regulatory elements, including prokaryotic and eukaryotic, constitutive and inducible promoters, and origins of replication can be found in, for example, MOLECULAR CLONING: A LABORATORY MANUAL (Joseph F. Sambrook and David W. Russell, eds.; 3rd Ed.; Vols. 1, 2, and 3; Cold Spring Harbor Laboratory Press; 2001) and MOLECULAR CLONING: A LABORATORY MANUAL (Michael R. Green and Joseph F. Sambrook, eds.; 4th Ed.; Vols. 1, 2, and 3; Cold Spring Harbor Laboratory Press; 2012).

In some embodiments, the lentiviral vector described herein can be structured and arranged so that the expression of the proteins, enzymes, and viral elements necessary for producing retroviral particles (i.e. cis-acting and trans-acting genes) are under the control of a transcriptional regulatory element. In a preferred embodiment, the lentiviral vector can further comprise a transcriptional regulatory element and the transcriptional regulatory element is upstream (i.e. in the 5′ direction) of the proteins, enzymes, and viral elements necessary for producing retroviral particles (i.e. cis-acting and trans-acting genes) and, optionally, the transcriptional regulatory element controls the expression (i.e. transcription or translation) of the nucleic acid encoding proteins, enzymes, and viral elements necessary for producing retroviral particles (i.e. cis-acting and trans-acting genes). In some embodiments, the transcriptional regulatory element may be constitutively active or may be a constitutive promoter.

In some embodiments, the lentiviral vectors described herein and nucleic acids encoding the heterologous envelope protein may be amplified or produced prior to the introduction into producer cells and, accordingly, prior to the production of viral particles. In some embodiment, the lentiviral vectors and nucleic acids encoding the other proteins, enzymes, and elements necessary for retroviral particle production may be amplified or produced prior to the introduction into producer cells, and, accordingly, the production of the retroviral proteins.

In some embodiments, the lentiviral vectors and nucleic acids encoding the heterologous envelope protein may be structured and arranged such that a transcriptional control element drives the transcription, and therefore translation, of the heterologous envelope protein in a producer cell to facilitate the production the lentiviral particles. In some embodiments, the lentiviral vectors and nucleic acids encoding the proteins, enzymes, viral elements (i.e. cis- and trans-acting genes, including rev and gag/pol) necessary for the production of the retroviral particles may be structured and arranged so that a transcriptional control element may drive the transcription, and therefore translation, of the proteins, enzymes, viral elements (i.e. cis- and trans-acting genes, including rev and gag/pol) in a producer cell so that the producer cell produces the retroviral particles.

In some embodiments of the manufacturing process disclosed herein, the apheresis product (e.g., the population of immune cells, the population of CD4⁺ and CD8⁺ cells, or the population of eukaryotic donor cells) is transfected with an effective dose of a lentiviral vector or retroviral vector comprising a chimeric antigen receptor (CAR), or an engineered TCR described. In some embodiments, the lentiviral vector is a lentiviral vector particle.

A. Lentiviruses

Vectors derived from retroviruses such as lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

Retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

In some embodiments, the lentiviral vector is based on a virus selected from the group consisting of a retroviral vector, an alpharetroviral vector, a betaretroviral vector, a gammaretroviral vector, a deltaretroviral vector, and an epsilonretroviral vector. In some embodiments, the lentiviral vector is based on a Human immunodeficiency virus (HIV), an Equine infectious anaemia virus (EIAV), a visna-maedi virus (VMV) virus, a caprine arthritis-encephalitis virus (CAEV), a feline immunodeficiency virus (FIV), a bovine immune deficiency virus (BIV), a VISNA virus, and a simian immunodeficiency virus (SIV). In one embodiment, the viral vector is derived from EIAV. EIAV has the simplest genomic structure of the lentiviruses.

Feline immunodeficiency virus (FIV) RNA encapsidation determinants have been shown to be discrete and non-continuous, comprising one region at the 5′ end of the genomic mRNA (R-U5) and another region that mapped within the proximal 311 nt of gag.

In some embodiments of the manufacturing process disclosed herein, the lentiviral vector comprises a heterologous viral envelope protein (Env) selected from the group consisting of VSV-G of the Indiana strain, VSV-G of the New Jersey strain, the Cocal vesiculovirus virus envelope protein, the Isfahan virus envelope protein, Chandipura virus envelope protein, Pyri virus envelope protein, a murine leukemia virus (MLV) envelope glycoprotein, a SVCV virus envelope protein, and variants thereof.

B. Pseudotyping Lentiviral Vectors

Viral envelope proteins (env) determine the range of host cells which can ultimately be infected and transformed by recombinant retroviruses generated from the cell lines. In the case of lentiviruses, such as FflV-1, FflV-2, SIV, FIV and EIV, the env proteins include gp41 and gp120. Preferably, the viral env proteins expressed by packaging cells of the present disclosure are encoded on a separate vector from the viral gag and pol genes.

Examples of retroviral-derived env genes which can be employed in the present disclosure include, but are not limited to, MLV envelopes, 10A1 envelope, BAEV, FeLV-B, RD114, SSAV, Ebola, Sendai, FPV (Fowl plague virus), and influenza virus envelopes. In some embodiments, the retroviral-derived env gene is selected from a gene encoding an envelope protein from an RNA virus selected from Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae). In some embodiments, the retroviral-derived env gene is selected from a gene encoding an envelope protein from a DNA viruses selected from Hepadnaviridae, Circoviridae, Parvoviridae, Papovaviridae, Adenoviridae, Herpesviridae, Poxyiridae, and Iridoviridae.

In some embodiments, the retroviral-derived env gene is selected from Alfalfa mosaic virus (AMV), Human papillomavirus (HPV), White spot syndrome virus (WDSV), Semliki Forest virus (SFV), Rabies, Avian leukosis virus (ALV), bovine immunodeficiency virus (BIV), Bovine leukemia virus (BLV), Epstein-Barr virus (EBV), squirrel monkey retrovirus (SMRV), Equine Infectious Anemia Virus (EIAV), feline leukemia virus (FeLV), Caprine arthritis and encephalitis virus (CAEV), Sin Nombre virus (SNV), Human T-cell lymphotropic virus (HTLV), Simian T-cell leukemia viruses (STLVs), Venezuelan Equine Encephalitis Virus (VEEV), Mason-Pfizer monkey virus (M-PMV), Avian carcinoma virus MH2, Avian encephalomyelitis virus (AEV), V-crk sarcoma virus CT10, and Respiratory syncytial virus (RSV).

In some embodiments, the envelope proteins for pseudotyping a lentivirus of the present disclosure include, but are not limited to any of the following virus to Influenza A such as H1N1, H1N2, H3N2 and H5N 1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephalitis causing virus.

In some embodiments, the lentiviral vector may be pseudotyped with any molecule of choice. In some embodiments, the lentiviral vector of the present disclosure is pseudotyped with an envelope glycoproteins (Env) selected from the group consisting of a murine leukemia virus (MLV), a chimeric envelope glycoprotein variant derived from MLV, a vesicular stomatitis virus G glycoprotein (VSV-G), a prototypic foamy virus (PFV) modified envelope, and a chimeric envelope glycoprotein variants derived from gibbon ape leukemia virus (GaLV).

In some embodiments, the lentiviral vector is pseudotyped with an envelope glycoproteins (Env)viral vector selected from the group consisting of a murine leukemia virus (MLV), a vesicular stomatitis virus (VSV) Indiana strain, VSV New Jersey strain, Cocal vesiculovirus virus, Chandipura virus, Piry virus, spring viremia of carp virus (SVCV), Sigma virus, infectious hematopoietic necrosis virus (IHNV), Mokola virus, rabies virus CVS virus, Isfahan virus, Alagoas virus, Calchaqui virus, Jurona vrus, La Joya virus, Maraba virus, Perinet virus, Yug Bugdanovac virus, a prototypic foamy virus (PFV), and gibbon ape leukemia virus (GaLV).

In some embodiments, the Env protein may be a modified Env protein such as a mutant or engineered Env protein. Modifications may be made or selected to introduce targeting ability or to reduce toxicity or for another purpose. The Env protein may be a modified Env protein such as a mutant or engineered Env protein. Modifications may be made or selected to introduce targeting ability or to reduce toxicity or for another purpose.

1. VSV-G

The envelope glycoprotein (G) of Vesicular stomatitis virus (VSV), a rhabdovirus, is an envelope protein that has been shown to be capable of pseudotyping certain enveloped viruses and viral vector virions. VSV-G's ability to pseudotype MoMLV-based retroviral vectors in the absence of any retroviral envelope proteins is known in the art. Any retroviral vectors may be successfully pseudotyped with VSV-G. These pseudotyped VSV-G vectors may be used to transduce a wide range of mammalian cells. Non-infectious retroviral particles can be made infectious by the addition of VSV-G. VSV-G pseudotyped vectors have been shown to infect not only mammalian cells, but also cell lines derived from fish, reptiles and insects. VSV-G protein can be used to pseudotype certain retroviruses because its cytoplasmic tail is capable of interacting with the retroviral cores.

The provision of a non-retroviral pseudotyping envelope such as VSV-G protein gives the advantage that vector particles can be concentrated to a high titre without loss of infectivity. In comparison, the VSV glycoprotein is composed of a single unit. VSV-G protein pseudotyping offers potential advantages for both efficient target cell infection/transduction and during manufacturing processes because the VSV glycoprotein is composed of a single unit and can withstand the shearing forces during ultracentrifugation. In contrast, retrovirus envelope proteins are apparently unable to withstand the shearing forces during ultracentrifugation because they consist of two non-covalently linked subunits, and the interaction between the subunits may be disrupted by the centrifugation. WO 2000/52188 describes the generation of pseudotyped retroviral vectors, from stable producer cell lines, having vesicular stomatitis virus-G protein (VSV-G) as the membrane-associated viral envelope protein, and provides a gene sequence for the VSV-G protein.

Pseudotyping can confer one or more advantages. For example, with the lentiviral vectors, the env gene product of HIV-I based vectors would restrict these vectors to infecting only cells that express a protein called CD4. But if the env gene in these vectors had been substituted with env sequences from other RNA viruses, then they may have a broader infectious spectrum.

2. Cocal Vesiculovirus Envelope Glycoprotein

The particles and Cocal vesiculovirus envelope glycoprotein (Cocal-G) have lower toxicity to cells producing them (i.e. “producer cells”) and higher transduction efficiencies of cells being infected by them (i.e. “target cells”). The Cocal vesiculovirus envelope glycoprotein have a higher titer of particles than compositions comprising other viral particles. Accordingly, lentiviral vector comprising the Cocal vesiculovirus envelope glycoprotein can be produced at a higher concentration. Furthermore, compositions comprising Cocal vesiculovirus envelope glycoprotein have higher titers of mature and immature particles, higher titers of infective particles, and higher titers of genetic information carried within the particles (e.g. CARs) when compared to envelop glycoprotein derived from non-Cocal vesiculovirus. In some embodiments, the lentiviral vector comprises a nucleotide sequence encoding the Cocal-G envelope protein. In some embodiments, the Cocal-G envelope protein is a Cocal-G protein variant.

3. Ross River Virus

Ross River Virus (RRV) is an alphavirus spread by mosquitoes which is endemic and epidemic in tropical and temperate regions of Australia. Antibody rates in normal populations in the temperate coastal zone tend to be low (6% to 15%) although sero-prevalence reaches 27 to 37% in the plains of the Murray Valley River system. In 1979 to 1980 RRV became epidemic in the Pacific Islands. The disease is not contagious between humans and is never fatal, the first symptom being joint pain with fatigue and lethargy in about half of patients (Fields Virology).

The Ross River viral envelope has been used to pseudotype a nonprimate lentiviral vector (FIV) and following systemic administration predominantly transduced the liver. The transduction efficiency of a lentiviral vector pseudotyped with the Ross River viral envelope was reported to be 20-fold greater than the transduction efficiency obtained with a VSV-G pseudotyped vector. Furthermore, a lentiviral vector pseudotyped with the Ross River viral envelope caused less cytotoxicity as measured by serum levels of liver enzymes suggestive of hepatotoxicity.

4. Baculovirus GP64

The baculovirus GP64 protein has been shown to be an attractive alternative to VSVG for viral vectors used in the large-scale production of high-titer virus required for clinical and commercial applications. Compared with VSVG, GP64 vectors have a similar broad tropism and similar native titers. Because, GP64 expression does not kill cells, 293T-based cell lines constitutively expressing GP64 can be generated. In some embodiments, the lentiviral vector comprises a nucleotide sequence encoding the baculovirus GP64 protein. In some embodiments, the baculovirus GP64 protein is a variant baculovirus GP64 protein.

5. Other Envelope Glycoproteins

The lentiviral vector of the present disclosure may be pseudotyped with at least a part of a rabies G protein or a mutant, variant, homologue or fragment thereof. Teachings on the rabies G protein, as well as mutants thereof, may be found in WO 1999/61639; EP 0445625. Other envelopes which give reasonable titre when used to pseudotype EIAV include Mokola, Rabies, Ebola and LCMV (lymphocytic choriomeningitis virus).

C. Lentiviral Vectors

In some embodiments of the manufacturing processes disclosed herein, the CAR T cell treatment method, the method of introducing a modification to a mononuclear cell, or the lentiviral vector of the present disclosure, the lentiviral vector is an infectious lentiviral vector or a lentiviral vector.

One aspect of the present disclosure provides a lentiviral vector comprising a polynucleotide sequence encoding at least one heterologous viral envelope protein originating from a virus; a polynucleotide sequence encoding at least one viral rev protein; a polynucleotide sequence encoding at least one viral gag protein and at least one viral pol protein; and a polynucleotide sequence encoding a chimeric antigen receptor or an engineered T cell receptor (TCR). In some embodiments, at least part of one or more regions of the viral genome essential for replication is mutated. In some embodiments, at least part of one or more regions of the viral genome essential for replication is selected from the group consisting of a rev gene, a gag gene, a pol gene, an integrase gene, a 5′ LTR, a 3′ LTR and combination thereof. In some embodiments, the mutation is selected from the group consisting of a deletion, an insertion, or a substitution.

1. Non-Replicating Vectors

In an exemplary retroviral vector of the present disclosure, at least part of one or more protein coding regions essential for replication may be removed from the virus. For example, gag/pol and env may be absent or not functional. This makes the viral vector replication-defective. Portions of the viral genome may also be replaced by a library encoding candidate nucleic acid binding sequences that are operably linked to a regulatory control region and a reporter gene in the vector genome in order to generate a vector comprising candidate nucleic acid binding sequences which is capable of transducing a target non-dividing cell and/or integrating its genome into a host genome.

In the genome of a replication-defective lentiviral vector, the sequences of gag/pol and/or env may be mutated, absent and/or not functional. In a typical lentiviral vector, at least part of one or more coding regions for proteins essential for virus replication may be removed from the vector. This makes the viral vector replication-defective. Portions of the viral genome may also be replaced by a nucleotide of interest in order to generate a vector comprising a nucleotide of interest that is capable of transducing a non-dividing target cell and/or integrating its genome into the target cell genome.

In some embodiments, the lentiviral or retroviral vectors of the present disclosure are non-integrative vectors and/or non-replicative. See e.g., WO 2006/010834 and WO 2007/071994. In one aspect of the disclosure, the lentiviral or retroviral vectors of the present disclosure can be incapable of autonomous replication and specific integration in transduced cells. In some embodiments, the lentiviral or retroviral vectors of the present disclosure comprise a recombinant genome comprising, a lentiviral encapsidation psi sequence, and a RNA nuclear export element, a transgene and possibly a promoter and/or a sequence favoring the nuclear import of RNA, between the LTR 5′ and 3′ lentiviral sequences. In some embodiments, the lentiviral vector comprises a mutation in at least part of one or more regions of the viral genome essential for replication. In that embodiment, the one or more regions are selected from the group consisting of a rev gene, a gag gene, a pol gene, an integrase gene, a 5′ LTR, a 3′ LTR and combination thereof. The mutation is selected from the group consisting of a deletion, an insertion, or a substitution.

In some embodiments, the lentiviral or retroviral vectors comprise and/or further comprise a mutated integrase that prevent the integration of the retroviral or lentiviral genome into the genome of a host cell. In some embodiments, the lentiviral or retroviral vectors comprise a modified pol sequence that generate a non-functional integrase.

In a further embodiment, the lentiviral vectors have the ability to deliver a sequence which is devoid of or lacking a viral RNA. In a further embodiment, a heterologous binding domain (heterologous to gag) located on the RNA to be delivered and a cognate binding domain on Gag or Gag Pol can be used to ensure packaging of the RNA to be delivered. Both of these vectors are described in WO 2007/072056. In some embodiments, the recombinant retroviruses are replication incompetent, meaning that a retrovirus cannot replicate once it leaves the packaging cell.

2. Self-Inactivating Vectors

In some embodiments, the lentiviral vector is a non-replicating, self-inactivating minimal lentiviral vector derived from Human immunodeficiency virus (HIV) or Equine infectious anaemia virus (EIAV) which may be pseudotyped with an env selected from the group consisting of VSV-G, Ebola, Flu-HA, Sendai virus envelope F or HN, baculovirus GP64, Rabies G, Cocal vesiculovirus envelope protein, or an alternative viral envelope protein.

A skilled artisan will appreciate how to modify the methods disclosed herein for use with different retroviruses. For example, in some embodiments, the HIV RREs and the polynucleotide region encoding HIV Rev can be replaced with N-terminal RGG box RNA binding motifs and a polynucleotide region encoding ICP27. In some embodiments, the polynucleotide region encoding HIV Rev can be replaced with one or more polynucleotide regions encoding adenovirus E1B 55-kDa and E4 Orf6. In some embodiments, the recombinant retroviruses can be adenoviruses, adeno-associated viruses, herpesviruses, cytomegaloviruses, poxviruses, avipox viruses, influenza viruses, vesicular stomatitis virus (VSV), or Sindbis virus.

In some embodiments, the retroviral vectors or lentiviral vectors disclosed herein are self-inactivating vectors. As used herein, the term “self-inactivating vector” refers to vectors in which the 3′ LTR enhancer promoter region (U3 region) has been modified (e.g., by deletion or substitution). A self-inactivating vector can prevent viral transcription beyond the first round of viral replication. Consequently, a self-inactivating vector can be capable of infecting and then integrating into a host genome (e.g., a mammalian genome) only once, and cannot be passed further. Accordingly, self-inactivating vectors can greatly reduce the risk of creating a replication-competent virus.

In some embodiments, the viral particles can be self-inactivating. A self-inactivating viral particle can prevent viral transcription beyond the first round of viral replication. Consequently, a self-inactivating particle is capable of infecting a cell and the genetic information therein is capable of integrating into a host genome (e.g., a mammalian genome). This integration and transducing can only occur once, and cannot be passed further. Accordingly, self-inactivating particles can greatly reduce the risk of creating a replication-competent virus.

A commonly used lentiviral vector system is the so-called third-generation self-inactivating system. Third-generation lentiviral vector systems can include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences that facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. Additionally, the transfer plasmid can be designed with a deletion of the 3′ LTF, rendering the virus “self-inactivating” (SIN).

Third-generation systems also generally include two “packaging plasmids” and an “envelope plasmid.” The “envelope plasmid” generally encodes an Env gene operatively linked to a promoter. In at least one embodiment of a third-generation system, the Env gene is VSV-G, Ebola env, Flu-HA, Sendai virus envelope F, HN, baculovirus GP64, Rabies G, Cocal vesiculovirus envelope protein, or a derivative thereof (e.g., a variant described herein), and the promoter is the CMV promoter. The third-generation system uses two packaging plasmids, one encoding Gag and Pol and the other encoding Rev as a further safety feature, which is an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

In some embodiments, the lentiviral vector is a third generation self-inactivating (SIN) vector and does not contain any viral proteins and is replication incompetent. In that embodiment, no infectious particles are produced by cells that have been transduced and/or transfected with the vector.

3. Regulatory Elements

In some embodiment, the retroviral or lentiviral vector described herein comprises a transcriptional regulatory elements. In some embodiments, the transcriptional regulatory element is a promoter selected from a eukaryotic promoter or a constitutive promoter. Physiologic promoters (e.g., an EF-1α promoter) can be less likely to induce integration mediated genotoxicity, and can abrogate the ability of the retroviral vector to transform stem cells. Other physiological promoters suitable for use in a retroviral or lentiviral vector are known to those of skill in the art and can be incorporated into exemplary embodiments of the nucleic acid vector. In some embodiment, the promoter is an elongation-factor-1-alpha promoter (EF-1α promoter). Use of an EF-1α promoter can increase the efficiency in expression of downstream transgenes (e.g., a TCR and/or CAR encoding nucleic acid sequence).

In some embodiments, the lentiviral or retroviral vector further comprises a non-requisite cis-acting sequence that can improve titers and gene expression. One non-limiting example of a non-requisite cis-acting sequence is the central polypurine tract and central termination sequence (cPPT/CTS) which is important for efficient reverse transcription and nuclear import. Other non-requisite cis-acting sequences are known to those of skill in the art and can be incorporated into the lentiviral or retroviral vector particle.

In some embodiments, the lentiviral or retroviral vector disclosed herein further comprises a posttranscriptional regulatory element. Posttranscriptional regulatory elements can improve RNA translation, improve transgene expression and stabilize RNA transcripts. One example of a posttranscriptional regulatory element is the woodchuck hepatitis virus posttranscriptional regulatory element (WPRE). Accordingly, in some embodiments a nucleic acid vector further comprises a WPRE sequence. Various posttranscriptional regulator elements are known to those of skill in the art and can be incorporated into the lentiviral or retroviral vector.

The lentiviral or retroviral vector disclosed herein can further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, the lentiviral or retroviral vector comprises a 3′ U3 deleted LTR, a non-functional LTR and/or lacks a functional 3′ or 5′ L TR. Accordingly, the lentiviral or retroviral vector disclosed herein can comprise any combination of the elements described herein to enhance the efficiency of functional expression of transgenes. For example, a lentiviral or retroviral vector can comprise a WPRE sequence, cPPT sequence, RRE sequence, 5′LTR, 3′ U3 deleted LTR′ in addition to a nucleic acid encoding for a TCR or CAR.

D. Lentiviral Production

One aspect of the present disclosure provides a method of generating a lentiviral or a retroviral vector particle described herein. Another aspect of the present disclosure provides a method of generating a lentiviral or a retroviral vector particle comprising introducing into a host cell the lentiviral or a retroviral vector particle disclosed herein.

Lentiviral vector production relies on the use of a “packaging cell line.” In general, the packaging cell line is a cell line whose cells are capable of producing infectious lentiviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a lentiviral vector system into lentiviral particles. In one embodiment, the present disclosure provides packaging cells that produce recombinant retrovirus, e.g., lentivirus, pseudotyped with the VSV-G glycoprotein or variant thereof as disclosed herein. Large scale viral particle production is often necessary to achieve a reasonable viral titer. Viral particles are produced by transfecting a transfer vector into a packaging cell line that comprises viral structural and/or accessory genes, e.g., gag, pol, env, tat, rev, vif, vpr, vpu, vpx, or nef genes or other retroviral genes.

1. Production of Retroviral Therapeutic Vectors

In some embodiments, the retroviral vector of the present disclosure may be produced by the transient transfection of HEK293T cells with four plasmids consisting of: (1) the recombinant retroviral vector genome plasmid encoding the required transgene(s) and a binding site that is capable of interacting with an RNA-binding protein; (2) the synthetic retroviral gag/pol expression plasmid; (3) the envelope (env) expression plasmid (e.g. VSV-G or variants thereof); (4) The RNA-binding protein expression plasmid.

In some embodiments, the retroviral vector of the present disclosure is HIV. In that embodiment, the retroviral vector may be produced by the transient transfection of HEK293T cells with five plasmids: (1) the recombinant HIV vector genome plasmid encoding the required transgene(s), a binding site that is capable of interacting with an RNA-binding protein, and the RRE sequence; (2) a synthetic gag/pol expression plasmid; (3) the envelope (env) expression plasmid (e.g. VSV-G, Cocal vesiculovirus or variants thereof); (4) The RNA-binding protein expression plasmid; (5) the REV expression plasmid.

In some embodiments, the retroviral vector of the invention may be produced by using packaging cells that stably express (1) gag/pol; (2) env (e.g. VSV-G or variants thereof); and (3) the RNA-binding protein, and, for HIV vectors, Rev, and a plasmid encoding the recombinant retroviral vector genome encoding the required transgene(s) (e.g., a CAR) and a binding site that is capable of interacting with the an RNA-binding protein, and for HIV vectors, includes the RRE sequence, is introduced into such cells by transient transfection.

In some embodiments, the retroviral vector of the present disclosure may be produced in producer cells that stably express (1) gag/pol, (2) env (e.g. VSV-G or variants thereof), (3) an RNA-binding protein, (4) a recombinant EIAV vector genome encoding the required transgene(s) (e.g., CAR) and a binding site that is capable of interacting with an RNA-binding protein.

In some embodiments, the lentiviral vector is an HIV lentiviral vector. In that embodiment, the HIV vector may be produced in producer cells that stably express (1) gag/pol; (2) env (e.g. VSV-G or variants thereof); (3) the RNA-binding protein; (4) the recombinant HIV vector genome encoding the required transgene(s) (e.g., CAR), a binding site that is capable of interacting with an RNA-binding protein, and the RRE sequence, and (5) REV.

a. Codon Optimization

In some embodiments, any of the polynucleotides used in the present disclosure for generating the lentiviral vector may be codon-optimized. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

Many viruses, including HIV and other lentiviruses, use a large number of rare codons and by changing these to correspond to commonly used mammalian codons, increased expression of a gene of interest, or packaging components in mammalian producer cells, can be achieved. Codon usage tables are known in the art for mammalian cells, as well as for a variety of other organisms.

Codon optimization of viral vector components has a number of other advantages. By virtue of alterations in their sequences, the nucleotide sequences encoding the packaging components of the viral particles required for assembly of viral particles in the producer cells/packaging cells have RNA instability sequences eliminated from them. At the same time, the amino acid sequence coding sequence for the packaging components is retained so that the viral components encoded by the sequences remain the same, or at least sufficiently similar that the function of the packaging components is not compromised. In lentiviral vectors codon optimization also overcomes the Rev/RRE requirement for export, rendering optimized sequences Rev-independent. Codon optimization also reduces homologous recombination between different constructs within the vector system (for example between the regions of overlap in the gag-pol and env open reading frames). The overall effect of codon optimization is therefore a notable increase in viral titer and improved safety.

In one embodiment only codons relating to instability sequences are codon optimized. In a preferred embodiment, the sequences are codon optimized in their entirety. In that embodiment, the sequence encompassing the frameshift site of gag-pol (see below). The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. Derivations from optimal codon usage may be made, for example, in order to accommodate convenient restriction sites, and conservative amino acid changes may be introduced into the Gag-Pol proteins.

In one embodiment, codon optimization is based on lightly expressed mammalian genes. Due to the degenerate nature of the genetic code, it will be appreciated that numerous gag-pol sequences can be achieved by a skilled worker. Also there are many retroviral variants described which can be used as a starting point for generating a codon-optimized gag-pol sequence. Lentiviral genomes can be quite variable. For example there are many quasi-species of HIV-1 which are still functional. This is also the case for EIAV. These variants may be used to enhance particular parts of the transduction process. Examples of HIV-1 variants may be found at the HIV Databases operated by Los Alamos National Security, LLC at hiv-web.lanl.gov. Details of EIAV clones may be found at the National Center for Biotechnology Information (NCBI) database located at ncbi.nlm.nih.gov.

The strategy for codon-optimized gag-pol sequences can be used in relation to any retrovirus. This would apply to all lentiviruses, including EIAV, FIV, BIV, CAEV, VMR, SIV, HIV-1 and HIV-2. In addition, this method could be used to increase expression of genes from HTLV-1, HTLV-2, HFV, HSRV and human endogenous retroviruses (HERV), MLV and other retroviruses.

Codon optimization can render gag-pol expression Rev-independent. In order to enable the use of anti-rev or RRE factors in the lentiviral vector, however, it would be necessary to render the viral vector generation system totally Rev/RRE-independent. Thus, the genome also needs to be modified. This is achieved by optimizing vector genome components. Advantageously, these modifications also lead to the production of a safer system absent of all additional proteins both in the producer and in the transduced cell.

b. Production of Viral Particles

The production of infectious viral particles and viral stock solutions may be carried out using conventional techniques as described herein. Recombinant viruses (e.g., retroviral or lentiviral vector or particle described herein) with titers of several millions of transducing units per milliliter (TU/mL) can be generated by known techniques. After ultracentrifugation concentrated stocks of about 10⁸ TU/mL, 10⁹ TU/mL, 10¹⁰ TU/mL, 10¹¹ TU/mL, or 10 TU/mL, or any intervening titer can be obtained. Recombinant viruses (e.g., retroviral or lentiviral vector or particle described herein) may be delivered according to viral titer (TU/mL), which can be measured, for example, by using a commercially available p24 titer assay, which is an ELISA against the p24 viral coat protein.

In some embodiments, the enriched apheresis product is transfected with a lentiviral vector or retroviral vector comprising a viral transducing unit (TU) per enriched apheresis product (cell) concentration of about 1×10⁸ to about 1×10¹⁰ TU/10⁸ cells, about 5×10⁸ to about 5×10⁹ TU/10⁸ cells, about 1×10⁹ to about 5×10⁹ TU/10⁸ cells, about 1×10⁹ to about 4×10⁹ TU/10⁸ cells, about 1×10⁹ to about 3×10⁹ TU/10⁸ cells, about 1×10⁹ to about 2×10⁹ TU/10⁸ cells, or any intervening TU thereof.

In one embodiment, the manufacturing process contemplated herein comprises transfecting the enriched apheresis product with a lentiviral vector or retroviral vector concentration of about 1×10⁸ TU/10⁸ cells, about 5×10⁸ TU/10⁸ cells, about 6×10⁸ TU/10⁸ cells, about 7×10⁸ TU/10⁸ cells, about 8×10⁸ TU/10⁸ cells, about 9×10⁸ TU/10⁸ cells, about 1×10⁹ TU/10⁸ cells, about 2×10⁹ TU/10⁸ cells, about 3×10⁹ TU/10⁸ cells, about 4×10⁹ TU/10⁸ cells, about 5×10⁹ TU/10⁸ cells, about 6×10⁹ TU/10⁸ cells, about 7×10⁹ TU/10⁸ cells, about 8×10⁹ TU/10⁸ cells, about 9×10⁹ TU/10⁸ cells, or about 1×10¹⁰ TU/10⁸ cells, or any intervening TU. In a particular embodiment, the enriched apheresis product is transfected with a lentiviral vector or retroviral vector concentration of about 1×10⁷ to about 2×10⁹ TU/10⁸ cells.

The production of viral particles and viral stock solutions may be carried out using conventional techniques. Methods of preparing viral stock solutions are known in the art and are illustrated by, e.g., Soneoka et al. (1995) Nucl. Acids Res. 23:628-633, and Landau et al. (1992)J. Virol. 66:5110-5113. Recombinant viruses with titers of several millions of transducing units per milliliter (TU/mL) can be generated by known techniques. After ultracentrifugation concentrated stocks of about 10⁸ TU/mL, 10⁹ TU/mL, 10¹⁰ TU/mL, 10¹¹ TU/mL, or 10¹² TU/mL, or any intervening titer can be obtained.

c. p24 Titer Assay

Viruses may be delivered according to viral titer (TU/mL), which can be measured, for example, by using a commercially available p24 titer assay. A p24 titer assay is an ELISA against the p24 viral coat protein. Assuming that there are approximately 2000 molecules of p24 per physical particle (PP) of lentivirus, the following formula can be used to calculate the pg/mL of p24: (2×10³)×(24×10³ Da of p24 per PP), 48×10⁶/Avogadro=(48×10⁶) I (6×10²³)=8×10¹⁷ g of p24 per PP, approximately 1 PP per 1×10¹⁶ g of p24, 1×10⁴ PP per pg of p24. In some embodiments, a reasonably well packaged, VSV-G pseudotyped lentiviral vector has an infectivity index in the range of about 1 TU per 1000 physical particles (PP) to about 1 TU per 100 PP (or less). Thus, the range of p24 is approximately about 10 to about 100 TU/pg. It is through this conversion that TU/mL is obtained.

Lentiviral titers may also be determined by analysis of transduced human osteosarcoma (HOS) cells. Briefly, transduced HOS cells are cultured for seven days in DMEM supplemented with 10% fetal bovine serum (FBS) after which the genomic DNA is extracted by DNeasy (Qiagen, Venlo Netherlands, Cat #69506) and evaluated by quantitative PCR (qPCR). The primer/probe sets of the qPCR protocol measures the vector copy number (VCN) of the transduced cells by determining the number of lentiviral psi-gag region copies per number of endogenous human RNaseP copies. The integrity of the provirus was assessed by sequencing individual pro viral insertions. In some embodiment, the viral titer is determined using a HOS cell line assay.

d. Host Cells

As used herein, a “host cell” is a cell transfected with a nucleic acid vector to replicate and produce more of the nucleic acid vector per se (i.e. more plasmid). In some embodiments, supernatants comprising lentiviral vector (LV) encoding a CAR or a TCR disclosed herein are produced in HEK 293T cells. To produce the lentiviral vector of the present disclosure, 293 cells are transiently transfected with 4-plasmids: a plasmid encoding HIV gag-pol, a plasmid encoding the VSV-G envelope protein, a plasmid encoding HIV rev protein, and a lentiviral transfer vector encoding a CAR.

Bacterial cells, yeast cells, and animal cells can be used for amplifying or producing the nucleic acid and vectors encoding the heterologous envelope protein or the proteins, enzymes, viral elements (i.e. cis- and trans-acting genes, including rev and gag/pol) necessary for the production of the retroviral particles. For amplification in a bacterial cell, suitable promoters include, but are not limited to, lacI, lacZ, T3, T7, gpt, lambda P and trc. For amplification in a eukaryotic cell or expression in a eukaryotic cell, suitable promoters include, but are not limited to, light or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters can be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (A1cR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

In some embodiments, the host cell and producer cells can be from the same cell lines. In some embodiments, the host cell and the producer cell are HEK293-T cells. Accordingly, in some embodiments, the promoter can be expressed generally in all cells, or selectively in the producer cells, or specifically in the producer cells. In some embodiments, the promoter is a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565. For expression in a yeast host cell for amplification, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (US 2004/0131637), a pagC promoter, a nirB promoter, and the like (see, e.g., Dunstan et al., Infect. Immun. (1999) 67:5133-5141; McKelvie et al., Vaccine (2004) 22:3243-3255); a sigma70 promoter, e.g., a consensus sigma70 promoter (see, e.g., GenBank Accession Nos. AX798980, AX798961, and AX798183); a stationary phase promoter, e.g., a dps promoter, an spy promoter, and the like; a promoter derived from the pathogenicity island SPI-2; an actA promoter; an rpsM promoter; a tet promoter; an SP6 promoter; and the like. Suitable strong promoters for use in prokaryotes such as Escherichia coli include, but are not limited to Trc, Tac, T5, T7, and PLambda.

One aspect of the present disclosure provides a method of generating a lentiviral vector particle comprising introducing into a host cell the lentiviral vector described herein.

Another aspect of the present disclosure provides a method for delivering a nucleic acid encoding a chimeric antigen receptor (CAR), an engineered T Cell receptor, or a therapeutic protein to a cell, the method comprising introducing into the cell a transfer plasmid comprising: a polynucleotide sequence encoding at least one heterologous viral envelope protein engineered by the methods described herein; a polynucleotide sequence encoding at least one retroviral rev protein; a polynucleotide sequence encoding at least one retroviral gag protein and a retroviral pol protein; and/or a polynucleotide sequence encoding the chimeric antigen receptor, the engineered T cell receptor (TCR), or the therapeutic protein. In some embodiments, at least part of one or more regions of the retroviral genome essential for replication is mutated as described herein.

Another aspect of the present disclosure provides a lentiviral vector particle generated by the methods described herein.

Another aspect of the present disclosure provides a method of introducing a modification to a cell, the method comprising electroporating a cell with an effective dose of the lentiviral vector particle described herein, thereby generating a modified cell. In some embodiments, the cell is contacted with the effective dose of the lentiviral vector prior to electroporation (e.g., application of electricity). Alternatively, the cell may be contacted with the effective dose of the lentiviral vector for up to about 4 hours after electroporation (e.g., application of electricity). For example, the cell may be contacted with the effective dose of the lentiviral vector for at least about 5-30 minutes, at least about 25-50 minutes; at least about 5-60 minutes, at least about 5-12 minutes, at least about 60-120 minutes, at least about 120-240 minutes after electroporation. Alternatively, the cell may be contacted with the effective dose of the lentiviral vector for at least about 1 minute, at least about 2 minutes, at least about 5 minutes, at least about 10 minutes, at least about 20 minutes, at least about 30 minutes, at least about 45 minutes, at least about 60 minutes, at least about 75 minutes, at least about 90 minutes, at least about 100 minutes, at least about 110 minutes, at least about 120 minutes, at least about 150 minutes, at least about 160 minutes, at least about 170 minutes, at least about 180 minutes, at least about 190 minutes, at least about 200 minutes, at least about 220 minutes, or at least about 240 minutes after electroporation.

Adding the lentiviral vector to the cell up to 4 hrs after electroporation (e.g., 1 minute to 2 hrs, or 1 minutes to 4 hrs) can reduce the amount of lentiviral particles that are killed by the electroporation, which can be toxic to the lentiviral particles. Accordingly, adding the lentiviral vector to the cell up to 4 hrs after electroporation can enhance CAR transfection. For example, addition of the lentiviral vector to the cell up to 4 hrs after electroporation can enhance CAR expression by about 10-15% when compared to a traditional electroporation process (e.g., adding the lentiviral to the cell before electroporation).

In some embodiments, the cell is selected from the group consisting of immune cells, eukaryotic donor cells, mononuclear cells, enriched lymphocytes, B lymphocytes, T lymphocytes, CD4⁺ T lymphocytes, CD8⁺ T lymphocytes, dendritic cells, monocytes, natural killer (NK) cells, natural killer T (NKT) cells, T-regulatory cells, CD4⁺ T-helper cells, CD8⁺ cytotoxic T lymphocytes (CTLs), CD62L⁺ cells, CD27⁺ cells, CCR7⁺ cells, CD45RO⁻ cells, CD45RA⁺ cells, neutrophils, basophils, eosinophils, megakaryocytes, stem cells, hematopoietic stem cells (HSCs), hematopoietic progenitor cells (HPCs), CD34⁺ cells, CD34⁺ peripheral blood stem cells, lymphokine-activated killer cells (LAKs), tumor infiltrating lymphocytes (TILs), circulating tumor specific T cells, mesenchymal stem cells, mast cells, a monocyte, a macrophage, a neutrophil, a basophil, an eosinophil, a dendritic cell, a megakaryocyte, and combinations thereof.

In some embodiments, the cell can be a lymphoid cell selected from the group consisting of a T cell, a B cell, a natural killer (NK) cell, a CD8⁺ T cell, a CD4⁺ T cell, a cytotoxic T lymphocyte, a regulatory T cell, and any combination thereof. In some embodiments, the cell can be a myeloid cell selected from the group consisting of a monocyte, a macrophage, a neutrophil, a basophil, an eosinophil, a dendritic cell, a megakaryocyte, and any combination thereof. In some embodiments, the cell can be a stem cell, a hematopoietic stem cell, an hematopoietic progenitor cell, a CD34⁺ cell, or CD34⁺ peripheral blood stem cell.

In some embodiments of the method of introducing a modification to a cell, the method comprises electroporating a cell with an effective dose of the lentiviral vector particle and the effective doses comprises about 0.5, about 1, about 1.5, about 2, about 2.5, about 3, about 3.5, about 4, about 5, about 6, about 7, about 8, about 9, about 10, about 15, or about 20 μl of the lentiviral vector.

In some embodiments, the effective dose of the lentiviral vector particle comprises a multiplicity of infection (MOI) of about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.25, about 1.5, about 2.0, about 3.0, about 4.0, or about 5.0. In some embodiments, the effective dose of the lentiviral vector particle comprises about 2 μl of lentiviral vector particle at a MOI of about 0.08. In some embodiments, the effective dose of the lentiviral vector particle comprises about 5 μl of lentiviral vector particle at a MOI of about 0.2. In some embodiments, the effective dose of the lentiviral vector particle comprises about 10 μl of lentiviral vector particle at a MOI of about 0.4.

E. Method of Producing Therapeutic Proteins

One aspect of the present disclosure provides a method of producing a therapeutic protein, the method comprising manufacturing a population of engineered immune cells, or a population of engineered eukaryotic cells comprising the therapeutic protein using the method of described herein; harvesting the therapeutic protein; and isolating and purifying the therapeutic protein. In some embodiments, the therapeutic protein is selected from the group consisting of enzymes, regulatory proteins, receptors, peptides, peptide hormones, cytokines, membrane or transport proteins, vaccine antigens, antigen-binding proteins, immune stimulatory proteins, allergens, full-length antibodies or antibody fragments or derivatives; single chain antibodies, (scFv), Fab fragments, Fv fragments, single domain antibodies (VH or VL fragment), domain antibodies, camelid single domain antibodies (VHH), nanobodies, and a combination thereof.

### IV. Chimeric Receptors

One aspect of the present disclosure provides a method for manufacturing a population of engineered immune cells, the method comprising: enriching a population of lymphocytes, a population immune cells, or a population of CD4⁺ and CD8⁺ cells from blood obtained from a subject; admixing the population of lymphocytes, the population of immune cells or the population of CD4⁺ and CD8⁺ cells with one or more buffer solutions; and transfecting the population of lymphocytes, the population of immune cells, or the population of CD4⁺ and CD8⁺ cells with an effective dose of a modifying agent; thereby generating a population of modified lymphocytes, a population of modified immune cells, or a population of modified CD4⁺ and CD8⁺ cells.

Another aspect of the present disclosure provides a method for manufacturing a population of engineered immune cells, the method comprising: enriching a population of lymphocytes, a population of immune cells or a population of CD4⁺ and CD8⁺ cells from a donor leukapheresis; admixing the population of lymphocytes, the population of immune cells or the population of CD4⁺ and CD8⁺ cells with one or more buffer solutions; transfecting the population of lymphocytes, the population of immune cells, or the population of CD4⁺ and CD8⁺ cells with an effective dose of a modifying agent; thereby generating a population of modified lymphocytes, a population of modified immune cells or a population of modified CD4⁺ and CD8⁺ cells.

Yet, another aspect of the present disclosure provides a method for manufacturing a population of engineered eukaryotic cells, the method comprising: obtaining a population of eukaryotic donor cells from a subject; admixing the population of eukaryotic donor cells with one or more buffer solutions; and transfecting the population of eukaryotic donor cells with an effective dose of a modifying agent, thereby generating a population of modified eukaryotic donor cells. In some embodiments, the transfected cells are cultured and expanded in the presence of one or more stimulating agents.

In some embodiments, the cells are transfected with one or more modifying agents selected from the group consisting of a small molecule agent, a biologic agent, a therapeutic, a protein, a peptide, a protein therapeutic, a peptide therapeutic, a nucleic acid, DNA, RNA, mRNA, a chimeric antigen receptor, a heterologous T cell receptor, a retroviral vector, a lentiviral vector, an adenoviral vector, and an adeno-associated viral vector.

In some embodiments, the lentiviral vector or retroviral vector comprises a nucleotide sequence encoding a chimeric antigen receptor (CAR); an engineered T cell receptor; and/or a nucleic acid sequence encoding a polypeptide that enhances the immune cell function, or a functional derivative thereof.

In some embodiments, the lentiviral vector or retroviral vector comprises a nucleic acid encoding a chimeric antigen receptor (CAR), an engineered T cell receptor (TCR), a Killer cell immunoglobulin-like receptor (KIR), an antigen-binding polypeptide, a cell surface receptor ligand, or a tumor antigen. In some embodiments, the nucleic acid encodes a chimeric antigen receptor (CAR). In some embodiments, the nucleic acid encodes an antigen-binding polypeptide. In some embodiments, the nucleic acid encodes a Killer cell immunoglobulin-like receptor (KIR). In additional embodiments, the exogenous nucleic acid encodes a cell surface receptor ligand or a tumor antigen.

In some embodiments, the retroviral vector or lentiviral vector can be used to introduce the TCR or CAR into an immune cell or precursor thereof (e.g., a T cell). In some embodiments, the retroviral vector or lentiviral vector particle can comprise additional elements that will aid in the functional expression of the TCR or CAR encoded therein. In some embodiments, an expression vector comprising a nucleic acid encoding for a TCR or CAR further comprises a mammalian promoter.

A. Chimeric Antigen Receptor

The present invention provides engineered immune effector cells (for example, T cells or NK cells) comprising one or more CARs that direct the immune effector cells to cancer. In some embodiments, the CAR comprises an antigen-binding domain, a transmembrane domain, a co-stimulatory domain, and an intracellular domain. The CAR may comprise any antigen binding domain, any hinge, any transmembrane domain, any costimulatory domain, and any intracellular signaling domain described herein.

The antigen binding domain may be operably linked to another domain of the CAR, such as the transmembrane domain or the intracellular domain, both described herein, for expression in any immune cell described herein. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present invention. A subject CAR of the present invention may also include a spacer domain as described herein. In some embodiments, each of the antigen binding domain, transmembrane domain, and intracellular domain is separated by a linker.

1. Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g. a tumor associated antigen) on a target cell (e.g. a cancer cell). The target antigen may include any type of protein, or epitope thereof, associated with the target cell. For example, the CAR may comprise affinity to a target antigen on a target cell that indicates a particular status of the target cell.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof. In some embodiments, the antigen binding domain comprises a full-length antibody. In some embodiments, the antigen binding domain comprises an antigen binding fragment (Fab), e.g., Fab, Fab′, F(ab′)2, a monospecific Fab2, a bispecific Fab2, a trispecific Fab2, a single-chain variable fragment (scFv), dAb, tandem scFv, VhH, V-NAR, camelid, diabody, minibody, triabody, or tetrabody. In some embodiments, the antigen-binding domain is selected from the group consisting of (a) a full-length antibody or antigen-binding fragment thereof, (b) a Fab, (c) a single-chain variable fragment (scFv), and (d) a single-domain antibody.

In some embodiments, a CAR of the present disclosure may have affinity for one or more target antigens on one or more target cells. In some embodiments, a CAR may have affinity for one or more target antigens on a single target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through a polypeptide linker, an Fc hinge region, or a membrane hinge region.

In some instances, the antigen binding domain may be derived from the same species in which the CAR will ultimately be used. For example, for use in humans, the antigen binding domain of the CAR may comprise a human antibody as described elsewhere herein, or a fragment thereof.

Accordingly, a CAR encoded by a lentiviral vector or retroviral vector of the present disclosure may target one of the following cancer associated antigens (tumor antigens): CD19; CD20; CD22 (Siglec 2); CD37; CD 123; CD22; CD30; CD 171; CS-1 (also referred to as CD2 subset 1, CRACC, SLAMF7, CD319, and 19A24); C-type lectin-like molecule-1 (CLL-1 or CLECL1); CD33; CD133; epidermal growth factor receptor (EGFR); epidermal growth factor receptor variant III (EGFRvIII); human epidermal growth factor receptor (HER1); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); Folate receptor alpha; Receptor tyro sine-protein kinase ERBB2 (Her2/neu); Mucin 1, cell surface associated (MUC 1); GalNAcal-O-Ser/Thr (Tn) MUC 1 (TnMUC1); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); tyrosine-protein kinase Met (c-Met); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-2 (GPC2); Glypican-3 (GPC3); NKG2D; KRAS; GDNF family receptor alpha-4 (GFRa4); IL13Ra2; Fc receptor-like 5 (FCRLS); and immunoglobulin lambda-like polypeptide 1 (IGLL1).

In some embodiments, the CAR targets CD19, CD20, CD22, BCMA, CD37, Mesothelin, PSMA, PSCA, Tn-MUC1, EGFR, EGFRvIII, c-Met, HER1, HER2, CD33, CD133, GD2, GPC2, GPC3, NKG2D, KRAS, or WT1. In some embodiments, the antigen-binding domain specifically binds a target antigen selected from the group consisting of CD4, CD19, CD20, CD22, BCMA, CD123, CD133, EGFR, EGFRvIII, mesothelin, Her2, PSMA, CEA, GD2, IL-13Ra2, glypican-3, GPC2, TnMuc1, CIAX, LI-CAM, CA 125, CTAG1B, Mucin 1, and Folate receptor-alpha.

2. Transmembrane Domain

A CAR encoded by a lentiviral vector or retroviral vector of the present disclosure can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen-binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

In some embodiments, the transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. In some embodiments, the transmembrane domain of particular use in this invention includes, without limitation, a transmembrane domain derived from (the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, TLR9, and a killer immunoglobulin-like receptor (KIR).

In some embodiments, the transmembrane domain comprises at least a transmembrane region of a protein selected from the group consisting of the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, TLR9, and a killer immunoglobulin-like receptor (KIR).

In some embodiments, the transmembrane domain may be synthetic. In some embodiments, the synthetic transmembrane domain comprises predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

In one embodiment, the transmembrane domain comprises a CD8a transmembrane domain. In some embodiments, the transmembrane domain comprises a CD8a transmembrane domain comprising the amino acid sequence set forth in SEQ ID NO: 33. In some embodiments, the transmembrane domain comprises the nucleotide sequence set forth in SEQ ID NO: 34.

In some embodiments, the transmembrane domain comprises a CD28 transmembrane domain. In some embodiments, the CAR comprises a CD28 transmembrane domain comprising the amino acid sequence set forth in SEQ ID NO: 37. In some embodiments, the CD28 transmembrane domain comprises the nucleotide sequence set forth in SEQ ID NO: 38.

Tolerable variations of the transmembrane and/or hinge domain will be known to those of skill in the art, while maintaining its intended function. In some embodiments, the transmembrane domain comprises an amino acid sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the amino acid sequences set forth in SEQ ID NOs: 33 and/or 37. In some embodiments the transmembrane domain is encoded by a nucleic acid sequence comprising the nucleotide sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the nucleotide sequences set forth in SEQ ID NOs: 34 and/or 38. The transmembrane domain may be combined with any hinge domain and/or may comprise one or more transmembrane domains described herein.

In some embodiments, the CAR comprises: any transmembrane domain selected from the group consisting of the transmembrane domain of alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, TLR9, and a killer immunoglobulin-like receptor (KIR); any costimulatory signaling domains, and any intracellular domains or cytoplasmic domains described herein, or any of the other domains described herein that may be included in the CAR, and optionally a hinge domain.

In some embodiments, the CAR further comprises a spacer domain between the extracellular domain and the transmembrane domain of the CAR, or between the intracellular domain and the transmembrane domain of the CAR. In some embodiments, the spacer domain may be a short oligo- or polypeptide linker, e.g., between about 2 and about 10 amino acids in length. For example, glycine-serine doublet provides a particularly suitable linker between the transmembrane domain and the intracellular signaling domain of the subject CAR. Accordingly, the CAR of the present disclosure may comprise any of the transmembrane domains, hinge domains, or spacer domains described herein.

3. Hinge Domain

In some embodiments, a CAR encoded by a lentiviral vector or retroviral vector of the present disclosure further comprises a hinge region. The hinge region of the CAR is a hydrophilic region which is located between the antigen binding domain and the transmembrane domain. In some embodiments, the hinge domain facilitates proper protein folding for the CAR. In some embodiments, the hinge domain is an optional component for the CAR. In some embodiments, the hinge domain comprises a domain selected from Fc fragments of antibodies, hinge regions of antibodies, CH2 regions of antibodies, CH3 regions of antibodies, artificial hinge sequences or combinations thereof. In some embodiments, the hinge domain is selected from but not limited to, a CD8a hinge, artificial hinges made of polypeptides that may be as small as, three glycines (Gly). In some embodiments, the hinge region is a hinge region polypeptide derived from a receptor. In some embodiments, the hinge region is a CD8-derived hinge region). In one embodiment, the hinge domain comprises an amino acid sequence derived from human CD8, or a variant thereof. In some embodiments, a subject CAR comprises a CD8a hinge domain and a CD8a transmembrane domain. In some embodiment, the CD8a hinge domain comprises the amino acid sequence set forth in SEQ ID NO: 35. In some embodiments, the CD8a hinge domain comprises the nucleotide sequence set forth in SEQ ID NO: 36.

In some embodiments the hinge domain comprises an amino acid sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the amino acid sequences set forth in SEQ ID NO: 35.

In some embodiments the hinge domain is encoded by a nucleic acid sequence comprising the nucleotide sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the nucleotide sequences set forth in SEQ ID NO: 36.

In some embodiments, the hinge domain connects the antigen-binding domain to the transmembrane domain, which, is linked to the intracellular domain. In exemplary embodiments, the hinge region is capable of supporting the antigen binding domain to recognize and bind to the target antigen on the target cells. In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

In some embodiments, the hinge domain has a length selected from about 4 to about 50, from about 4 to about 10, from about 10 to about 15, from about 15 to about 20, from about 20 to about 25, from about 25 to about 30, from about 30 to about 40, or from about 40 to about 50 amino acids. Suitable hinge regions can be readily selected and can be of any of a number of suitable lengths, such as from about 1 amino acid (e.g., Glycine (Gly) to about 20 amino acids, from about 2 to about 15, from about 3 to about 12 amino acids, including about 4 to about 10, about 5 to about 9, about 6 to about 8, or about 7 to about 8 amino acids, and can be about 1, about 2, about 3, about 4, about 5, about 6, or about 7 amino acids.

In some embodiments, the amino acid is a glycine (Gly). Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. In some embodiment, the hinge regions comprises glycine polymers (G)n, glycine-serine polymers. In some embodiments, the hinge region comprises glycine-serine polymers selected from the group consisting of (GS)n, (GSGGS)n and (GGGS)n, where n is an integer of at least one). In some embodiments, the hinge domain comprises an amino acid sequence of including, but not limited to, GGSG (SEQ ID NO: 24), GGSGG (SEQ ID NO: 25), GSGSG (SEQ ID NO: 26), GSGGG (SEQ ID NO: 27), GGGSG (SEQ ID NO: 28), GSSSG (SEQ ID NO: 29). In some embodiment, the hinge region comprises glycine-alanine polymers, alanine-serine polymers, or other flexible linkers known in the art.

In some embodiments, the hinge region is an immunoglobulin heavy chain hinge region. Immunoglobulin hinge region amino acid sequences are known in the art. In some embodiments, an immunoglobulin hinge domain comprises an amino acid sequence selected from the group consisting of DKTHT (SEQ ID NO: 39); CPPC (SEQ ID NO: 40); CPEPKSCDTPPPCPR (SEQ ID NO: 41) (see, e.g., Glaser et al., J. Biol. Chem. (2005) 280:41494-41503); ELKTPLGDTTHT (SEQ ID NO: 42); KSCDKTHTCP (SEQ ID NO: 43); KCCVDCP (SEQ ID NO: 44); KYGPPCP (SEQ ID NO: 45); EPKSCDKTHTCPPCP (SEQ ID NO: 46) (human IgG1 hinge); ERKCCVECPPCP (SEQ ID NO: 47) (human IgG2 hinge); ELKTPLGDTTHTCPRCP (SEQ ID NO: 48) (human IgG3 hinge); SPNMVPHAHHAQ (SEQ ID NO: 49) (human IgG4 hinge); and the like.

In some embodiments, the hinge region is an immunoglobulin heavy chain hinge region. In some embodiments, the hinge is selected from CH1 and CH3 domains of IgGs (such as human IgG4). In some embodiments, the hinge domain comprises an amino acid sequence of a human IgG1, IgG2, IgG3, or IgG4 hinge domain. In some embodiments, the hinge region can include one or more amino acid substitutions and/or insertions and/or deletions compared to a wild-type (naturally-occurring) hinge region. In some embodiment, histidine at position 229 (His229) of human IgG1 hinge is substituted with tyrosine (Tyr). In some embodiments, the hinge domain comprises the amino acid sequence EPKSCDKTYTCPPCP (SEQ ID NO: 46).

4. Intracellular Domain

A CAR encoded by a lentiviral vector or retroviral vector of the present disclosure also comprises an intracellular domain. The intracellular domain or otherwise the cytoplasmic domain of the CAR is responsible for activation of the cell in which the CAR is expressed. The term “intracellular domain” is thus meant to include any portion of the intracellular domain sufficient to transduce the activation signal. In one embodiment, the intracellular domain includes a domain responsible for an effector function. The term “effector function” refers to a specialized function of a cell. Effector function of a T cell, for example, may be cytolytic activity or helper activity including the secretion of cytokines. In one embodiment, the intracellular domain of the CAR includes a domain responsible for signal activation and/or transduction. The intracellular domain may transmit signal activation via protein-protein interactions, biochemical changes or other response to alter the cell's metabolism, shape, gene expression, or other cellular response to activation of the chimeric intracellular signaling molecule.

Examples of an intracellular domain for use in the invention include, but are not limited to, the cytoplasmic portion of a T cell receptor (TCR), and any co-stimulatory molecule, or any molecule that acts in concert with the TCR to initiate signal transduction in the T cell, following antigen receptor engagement, as well as any derivative or variant of these elements and any synthetic sequence that has the same functional capability.

In certain embodiments, the intracellular domain comprises an intracellular signaling domain. Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma R11a, DAP10, DAP12, T cell receptor (TCR), CD2, CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11a, LFA-1, ITGAM, CD lib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, syk family tyrosine kinases (Syk, ZAP 70, etc.), src family tyrosine kinases (Lck, Fyn, Lyn, etc.), other co-stimulatory molecules described herein, any derivative, variant, or fragment thereof, any synthetic sequence of a co-stimulatory molecule that has the same functional capability, and any combination thereof.

In some embodiments, the intracellular signaling domain comprises an intracellular domain selected from the group consisting of cytoplasmic signaling domains of a human CD2, CD3 zeta chain (CD3), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof. In some embodiments, the intracellular signaling domain comprises CD3 zeta intracellular signaling domain.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors. Additionally, intracellular signaling domains may include signaling domains used by NK and NKT cells such as signaling domains of NKp30 (B7-H6), and DAP 12, NKG2D, NKp44, NKp46, DAP10, and CD3z.

Intracellular signaling domains suitable for use in the CAR of the present invention include any desired signaling domain that transduces a signal in response to the activation of the CAR (i.e., activated by antigen and dimerizing agent). In some embodiments, a distinct and detectable signal e.g. comprises increased production of one or more cytokines by the cell; change in transcription of a target gene; change in activity of a protein; change in cell behavior (e.g., cell death); cellular proliferation; cellular differentiation; cell survival; and/or modulation of cellular signaling responses. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in the CAR of the present invention include immunoreceptor tyrosine-based activation motif (ITAM)-containing intracellular signaling polypeptides. In some embodiments, the intracellular signaling domain includes at least one, at least two, at least three, at least four, at least five, or at least six ITAM motifs as described below. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, Fc gamma RI, Fc gamma RIIA, Fc gamma RIIC, Fc gamma RIIIA, FcRL5.

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcR gamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-zeta, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). In one embodiment, an intracellular signaling domain suitable for use in the CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the costimulatory signaling domains described herein, any of the antigen binding domains described herein, any of the transmembrane domains described herein, or any of the other domains described herein that may be included in the CAR. In some embodiment, the intracellular domain of the CAR comprises dual signaling domains. The dual signaling domains may include a fragment or domain from any of the molecules described herein. In some embodiments, the intracellular domain comprises 4-1BBcostimulatory domain and CD3 zeta signaling domain; CD28 costimulatory domain and CD3 zeta signaling domain; CD2 costimulatory domain and CD3 zeta signaling domain. In some embodiments, the intracellular domain of the CAR includes any portion of a co-stimulatory molecule, such as at least one signaling domain from CD3, CD27, CD28, ICOS, 4-1BB, PD-1, T cell receptor (TCR), any derivative or variant thereof, any synthetic sequence thereof that has the same functional capability, and any combination thereof.

Further, variant intracellular signaling domains suitable for use in a subject CAR are known in the art. The YMFM motif is found in ICOS and is a SH2 binding motif that recruits both p85 and p50alpha subunits of PI3K, resulting in enhanced AKT signaling. In one embodiment, a CD28 intracellular domain variant may be generated to comprise a YMFM motif.

In one embodiment, the intracellular domain of a subject CAR comprises a CD3 zeta intracellular signaling domain comprising the amino acid sequence set forth in SEQ ID NO: 50 or SEQ ID NO: 51, which may be encoded by a nucleic acid sequence comprising the nucleotide sequence set forth in SEQ ID NO: 52 or SEQ ID NO: 53, respectively.

Tolerable variations of the intracellular domain will be known to those of skill in the art, while maintaining specific activity. In some embodiments, the intracellular domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 50 or 51. In some embodiments, the intracellular domain is encoded by a nucleic acid sequence comprising a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleotide sequences set forth in SEQ ID NO: 52 or 53.

5. Costimulatory Domain

In some embodiments, the intracellular domain comprises a costimulatory signaling domain and an intracellular signaling. In certain embodiments, the intracellular domain comprises a costimulatory signaling domain. In one embodiment, the intracellular domain of the CAR comprises a costimulatory signaling domain selected from the group consisting of a portion of a signaling domain from proteins in the TNFR superfamily, CD27, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR, any derivative or variant thereof, any synthetic sequence thereof that has the same functional capability, and any combination thereof.

In some embodiments, the costimulatory domain comprises one or more of a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CDS, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof. In some embodiments, the costimulatory domain comprises one or more of a costimulatory domain of a protein selected from the group consisting of proteins in the CD28, 4-1BB (CD137), OX40 (CD134), CD27, CD2, or a combination thereof. In some embodiments, the costimulatory signaling domain comprises 4-1BB costimulatory domain. In some embodiments, the costimulatory signaling domain comprises CD2 costimulatory domain. In some embodiments, the costimulatory signaling domain comprises CD28 costimulatory domain.

In some embodiments, the costimulatory domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 54, 57, 59, 61, 64, 66, 68, or 70. In some embodiments, the intracellular domain is encoded by a nucleic acid sequence comprising a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleotide sequences set forth in SEQ ID NO: 55, 56, 58, 60, 62, 63, 65, 67, 69, or 71.

In one embodiment, the intracellular domain of a subject CAR comprises an ICOS costimulatory domain and a CD3 zeta intracellular signaling domain. In one embodiment, the intracellular domain of a subject CAR comprises a CD28 costimulatory domain and a CD3 zeta intracellular signaling domain. In one embodiment, the intracellular domain of a subject CAR comprises a CD28 YMFM variant costimulatory domain and a CD3 zeta intracellular signaling domain. In one embodiment, the intracellular domain of a subject CAR comprises a CD27 costimulatory domain and a CD3 zeta intracellular signaling domain. In one embodiment, the intracellular domain of a subject CAR comprises a OX40 costimulatory domain and a CD3 zeta intracellular signaling domain. In one exemplary embodiment, the intracellular domain of a subject CAR comprises a 4-1BB costimulatory domain and a CD3 zeta intracellular signaling domain. In one exemplary embodiment, the intracellular domain of a subject CAR comprises a CD2 costimulatory domain and a CD3 zeta intracellular signaling domain.

B. Additional Antigen-Binding Polypeptides

In some embodiments, the modified T cell expresses an antigen-binding polypeptide, a cell surface receptor ligand, or a polypeptide that binds to a tumor antigen. In some instances, the antigen-binding domain comprises an antibody that recognizes a cell surface protein or a receptor expressed on a tumor cell. In some instances, the antigen-binding domain comprises an antibody that recognizes a tumor antigen. In some instances, the antigen-binding domain comprises a full length antibody or an antigen-binding fragment thereof, a Fab, a F(ab)2, a monospecific Fab2, a bispecific Fab2, a trispecific Fab2, a single-chain variable fragment (scFv), a diabody, a triabody, a minibody, a V-NAR, or a VhH.

C. Cell Surface Receptor Ligands

In some embodiments, a lentiviral vector or retroviral vector of the present disclosure further comprises a nucleic acid encoding a cell surface receptor ligand. In some instances, the ligand binds to a cell surface receptor expressed on a tumor cell. In some cases, the ligand comprises a wild-type protein or a variant thereof that binds to the cell surface receptor. In some instances, the ligand comprises a full-length protein or a functional fragment thereof that binds to the cell surface receptor. In some cases, the functional fragment comprises about 90%, about 80%, about 70%, about 60%, about 50%, or about 40% in length as compared to the full length version of the protein but retains binding to the cell surface receptor. In some cases, the ligand is a de novo engineered protein that binds to the cell surface receptor. Exemplary ligands include, but are not limited to, epidermal growth factor (EGF), platelet-derived growth factor (PDGF), or Wnt3A.

D. Tumor Antigens

In some embodiments, a lentiviral vector or retroviral vector of the present disclosure further comprises a nucleic acid encoding a polypeptide that binds to a tumor antigen. In some embodiments, the tumor antigen is associated with a hematologic malignancy. Exemplary tumor antigens include, but are not limited to, CD19, CD20, CD22, CD33/IL3Ra, ROR1, mesothelin, c-Met, PSMA, PSCA, Folate receptor alpha, Folate receptor beta, EGFRvIII, GPC2, Tn-MUC1, GDNF family receptor alpha-4 (GFRa4), fibroblast activation protein (FAP), and IL13Ra2. In some instances, the tumor antigen comprises CD19, CD20, CD22, BCMA, CD37, Mesothelin, PSMA, PSCA, Tn-MUC1, EGFR, EGFRvIII, c-Met, HER1, HER2, CD33, CD133, GD2, GPC2, GPC3, NKG2D, KRAS, or WT1. In some instances, the polypeptide is a ligand of the tumor antigen, e.g., a full-length protein that binds to the tumor antigen, a functional fragment thereof, or a de novo engineered ligand that binds to the tumor antigen. In some instances, the polypeptide is an antibody that binds to the tumor antigen.

E. Engineered T Cell Receptors

In some embodiments, the antigen binding domain of a CAR described herein can be grafted to one or more constant domains of a T cell receptor (“TCR”) chain (e.g., a TCR alpha or TCR beta chain), to create a chimeric TCR. Chimeric TCRs can signal through the TCR complex upon antigen binding. For example, an scFv as disclosed herein, can be grafted to the constant domain, or at least a portion of the extracellular constant domain, the transmembrane domain of a TCR chain. As another example, an antibody fragment, for example a VL domain as described herein, can be grafted to the constant domain of a TCR alpha chain. Such chimeric TCRs may be produced, for example, by methods known in the art (for example, Aggen et al, Gene Ther. 2012 April; 19(4):365-74).

F. Switch Receptors and Dominant Negative Receptors

In one aspect, a lentiviral vector or retroviral vector of the present disclosure further comprises a nucleic acid encoding a dominant negative receptor, a switch receptor, or a combination thereof. In some embodiments, the lentiviral vector or retroviral vector described herein comprises a chimeric antigen receptor (CAR), and/or a dominant negative receptor. In some embodiments, the lentiviral vector or retroviral vector comprises a CAR, and/or a switch receptor. In some embodiments, the lentiviral vector or retroviral vector described herein comprises an engineered TCR, and a switch receptor. In some embodiments, the lentiviral vector or retroviral vector described herein comprises an engineered TCR, and a dominant negative receptor. In some embodiments, the lentiviral vector or retroviral vector described herein comprises a KIR, and a switch receptor. In some embodiments, the lentiviral vector or retroviral vector described herein further comprises a KIR, and a dominant negative receptor.

1. Switch Receptors

The present disclosure provides quick and efficient manufacturing processes for engineering modified immune cells comprising a CAR, or an exogenous TCR and/or a switch receptor. In some embodiments, the CAR, the TCR and/or the switch receptor are encoded by one or more nucleic acids. In some embodiments, the lentiviral vector or retroviral vector disclosed herein comprises one or more nucleic acid sequence encoding the CAR, the TCR and/or the switch receptor. In some embodiments, the nucleic acid sequence encoding the CAR is operably linked to a nucleic acid sequence encoding the switch receptor. In some embodiments, the switch receptor can enhances the efficiency of the CAR or the CAR expressing cell.

Tumor cells generate an immunosuppressive microenvironment that serves to protect them from immune recognition and elimination. This immunosuppressive microenvironment can limit the effectiveness of immunosuppressive therapies such as CAR-T or TCR-T cell therapy. For example, the secreted cytokine Transforming Growth Factor β (TGF β) directly inhibits the function of cytotoxic T cells and additionally induces regulatory T cell formation to further suppress immune responses. T cell immunosuppression due to TGFβ in the context of prostate cancers has been previously demonstrated. To reduce the immunosuppressive effects of TGF on the immune cells can be modified to express an engineered TGFβR comprising the extracellular ligand-binding domain of the TGFβR fused to the intracellular signaling domain of, for example, Interleukin-12 receptor (IL12R; TGFβR-IL12R). Therefore, a modified immune cell comprising a switch receptor may bind a negative signal transduction molecule in the microenvironment of the modified immune cell, and convert the negative signal transduction signal of an inhibitory molecule may have on the modified immune cell into a positive signal that stimulate the modified immune cell. A switch receptor of the present disclosure may be designed to reduce the effects of a negative signal transduction molecule, or to convert the negative signal into a positive signal, by virtue of comprising an intracellular domain associated with the positive signal.

As used herein, the term “switch receptor” refers to a molecule designed to reduce the effect of a negative signal transduction molecule on a modified immune cell of the present invention. The switch receptor comprises: a first domain that is derived from a first polypeptide that is associated with a negative signal (a signal transduction that suppresses or inhibits a cell or T cell activation); and a second domain that is derived from a second polypeptide that is associated with a positive signal (a signal transduction signal that stimulate a cell or a T cell). In some embodiments, the protein associated with the negative signal is selected from the group consisting of CTLA4, PD-1, TGFβRII, BTLA, VSIG3, VSIG8, and TIM-3. In some embodiments, the protein associated with the positive signal is selected from the group consisting of CD28, 4-1BB, IL12Rβ1, IL12Rβ2, CD2, ICOS, and CD27.

In one embodiment, the first domain comprises at least a portion of the extracellular domain of the first polypeptide that is associated with a negative signal, and the second domain comprises at least a portion of the intracellular domain of the second polypeptide that is associated with a positive signal. As such, a switch receptor comprises an extracellular domain associated with a negative signal fused to an intracellular domain associated with a positive signal. In some embodiments, the switch receptor comprises an extracellular domain of a signaling protein associated with a negative signal, a transmembrane domain, and an intracellular domain of a signaling protein associated with a positive signal. In some embodiments, the transmembrane domain of the switch receptor is selected from the transmembrane of the protein associated with a negative signal or the transmembrane domain of the protein associated with the negative signal. In some embodiments, the transmembrane domain of the switch receptor is selected from a transmembrane domain of a protein selected from the group consisting of CTLA4, PD-1, VSIG3, VSIG8, TGFβRII, BTLA, TIM-3, CD28, 4-1BB, IL12Rβ1, IL12Rβ2, CD2, ICOS, and CD27.

In some embodiments, the switch receptor is selected from the group consisting of PD-1-CD28, PD-1A132L-CD28, PD-1-CD27, PD-1A132L-CD27, PD-1-4-1BB, PD-1A132L-4-1BB, PD-1-ICOS, PD-1A132L-ICOS, PD-1-IL12Rβ1, PD-1A132L-IL12Rβ1, PD-1-IL12Rβ2, PD-1A132L-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2.

2. Dominant Negative Receptors

The present disclosure provides a quick and efficient manufacturing process for engineering modified immune cells comprising a CAR, or an exogenous TCR and a dominant negative receptor. In some embodiments, the CAR, the TCR and/or the switch receptor are encoded by one or more nucleic acid, In some embodiments, the lentiviral vector or retroviral vector disclosed herein comprises one or more nucleic acid sequence encoding the CAR, the TCR and/or the dominant negative receptor. In some embodiments, the nucleic acid sequence encoding the CAR is operably linked to a nucleic acid sequence encoding the dominant negative receptor. In some embodiments, the dominant negative receptor enhances the efficiency of the CAR or the CAR expressing cell.

As used herein, the term “dominant negative receptor” refers to a molecule designed to reduce the effect of a negative signal transduction molecule (e.g., the effect of a negative signal transduction molecule on a modified immune cell of the present invention). A dominant negative receptor is a truncated variant of a wild-type protein associated with a negative signal. In some embodiments, the protein associated with a negative signal he protein associated with the negative signal is selected from the group consisting of CTLA4, PD-1, BTLA, TGFβRII, VSIG3, VSIG8, and TIM-3.

A dominant negative receptor of the present invention may bind a negative signal transduction molecule (e.g., CTLA4, PD-1, BTLA, TGFβRII, VSIG3, VSIG8, and TIM-3) by virtue of an extracellular domain associated with the negative signal, may reduce the effect of the negative signal transduction molecule. For example, a modified immune cell comprising a dominant negative receptor may bind a negative signal transduction molecule in the microenvironment of the modified immune cell, but this binding will not transduce this signal inside the cell to modify the activity of the modified T cell. Rather, the binding sequesters the negative signal transduction molecule and prevents its binding to endogenous receptor/ligand, thereby reducing the effect of the negative signal transduction molecule may have on the modified immune cell. As such, to reduce the immunosuppressive effects of certain molecule, immune cells can be modified to express a dominant negative receptor that is a dominant negative receptor.

In some embodiments, the dominant negative receptor comprises a truncated variant of a wild-type protein associated with a negative signal. In some embodiments, the dominant negative receptor comprises a variant of a wild-type protein associated with a negative signal comprising an extracellular domain, a transmembrane domain, and substantially lacking an intracellular signaling domain. In some embodiments, the dominant negative receptor comprises an extracellular domain of a signaling protein associated with a negative signal, and a transmembrane domain. In some embodiments, the dominant negative receptor is PD-1, CTLA4, BTLA, TGFβRII, VSIG3, VSIG8, or TIM-3 dominant negative receptor. In some embodiments, the dominant negative receptor is PD-1, or TGFβRII dominant negative receptor. Tolerable variations of the dominant negative receptor will be known to those of skill in the art, while maintaining its intended biological activity (e.g., blocking a negative signal and/or sequestering a molecule having a negative signal when expressed in a cell).

G. Chemokine and Cytokine as Immune Enhancing Factors for Improved Fitness

The present disclosure provides quick and efficient manufacturing processes for engineering modified immune cells comprising a CAR, or an exogenous TCR and/or an immune enhancing factor that improves the fitness of the engineered immune cells. In some embodiments, the immune enhancing factor or a functional derivative thereof is a polypeptide that enhances the immune cell function.

In some embodiments, a polypeptide that enhances the immune cell function, or a functional derivative thereof is selected from a chemokine, a chemokine receptor, a cytokine, a cytokine receptor, Interleukin-7 (IL-7), Interleukin-7 receptor (IL-7R), Interleukin-15 (IL-15), Interleukin-15 receptor (IL-15R), Interleukin-21 (IL-21), Interleukin-18 (IL-18), Interleukin-18 receptor (IL-18R), CCL21, CCL19, or a combination thereof. In some embodiments, a chemokine, a chemokine receptor, a cytokine, a cytokine receptor, IL-7, IL-7R, IL-15, IL-15R, IL-21, IL-18, IL-18R, C-C Motif Chemokine Ligand 21 (CCL21), or C-C Motif Chemokine Ligand 19 (CCL19) is an immune function-enhancing factor that improves the fitness of the claimed modified immune cell. Without wishing to be bound by theory, the addition of a nucleic acid encoding a chemokine, a chemokine receptor, a cytokine, a cytokine receptor, IL-7, IL-7R, IL-15, IL-15R, IL-21, IL-18, IL-18R, CCL21, or CCL19 to the modified immune cell of the present disclosure enhances the immunity-inducing effect and antitumor activity of the modified immune cell.

1. T Cell Infiltration

Without wishing to be bound by theory, interleukins and chemokines, may promote increase T cell priming and/or T cell infiltration in a solid tumor. For instance, in microsatellite stable colorectal cancers (CRCs) with low T cell infiltration, IL-15 promotes T cell priming. In some embodiments, the combination of a CAR and chemokine/interleukine receptor complex promotes T cell priming. Furthermore, IL-15 may induce NK cell infiltration. In some embodiments, response to an IL-15/IL-15RA complex can result in NK cell infiltration. In certain embodiments, the modified immune cell described herein further comprises an IL-15/IL-15Ra complex. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. In some embodiments, IL-15 stimulates Natural Killer cells to eliminate (e.g., kill) pancreatic cancer cells. In some embodiments, therapeutic response to a modified immune cell described herein further comprising IL-15/IL15Ra is associated with Natural Killer cell infiltration in an animal model of colorectal cancer. In some embodiments, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra.

The ineffectiveness of CAR T cell therapy against solid tumors is partially caused by the limited recruitment and accumulation of immune cells and CAR T cells in solid tumors. One approach to solve this problem is to engineer CAR T cells that mimic the function of T-zone fibroblastic reticular cells (FRC). The lymph node is responsible for detecting pathogens and immunogens. The T-zone contains three types of cells: (1) innate immunity cells such as dendritic cells, monocytes, macrophages, and granulocytes; (2) adaptive immunity cells, such as CD4 and CD8 lymphocytes, and (3) stromal cells (FRCs). These cells cooperate to mount an effective immune response against a pathogen by facilitating the activation, differentiation and maturation of CD4 T cells. FRCs are particularly important because they form a network that allows dendritic cells and T cells to travel throughout the lymph node, and attracts B cells. In particular, FRCs provide a network for: (i) the recruitment of naive T cells, B cells and dendritic cells to the lymph node by releasing two chemokines (CCL21 and CCL19); (ii) T cell survival by secreting IL-7, which is a survival factor particularly for naive T cells; and (iii) trafficking of CD4 T cells toward the germinal center (GC; a different part of the lymph node). Accordingly, a CAR armored with exogenous CCL21, or CCL19 and IL-7, will enhance the recruitment of T cells, B cells and dendritic cells to solid tumors. In some embodiments, the modified immune cells engineered by the method disclosed herein comprises a lentiviral vector or retroviral vector comprising a nucleic acid encoding an immune function-enhancing factor, and a CAR. In that embodiment, the nucleic acid encoding the immune function-enhancing factor is a nucleic acid encoding interleukin-7 and a nucleic acid encoding CCL19 or CCL21.

In some embodiments, the nucleic acid of the immune function-enhancing factor (i.e. chemokine, the chemokine receptor, the cytokine, the cytokine receptor, IL-7, IL-7R, IL-15, IL-15R, IL-21, IL-18, CCL21, or CCL19) is fused to a CAR. In some embodiments, the chemokine, the chemokine receptor, the cytokine, the cytokine receptor, IL-7, IL-7R, IL-15, IL-15R, IL-21, IL-18, CCL21, or CCL19 is fused to a CAR via a self-cleaving peptide, such as a P2A, a T2A, an E2A, or an F2A.

2. T cell priming (IL-18)

The present disclosure provides quick and efficient manufacturing processes for engineering modified immune cells comprising a CAR, or an exogenous TCR and/or polypeptide which enhances T cell priming (i.e., T cell priming polypeptide). In some embodiments, the polypeptide that enhances T cell priming (ETP) is selected from the group consisting of a costimulatory molecule, a soluble cytokine, a polypeptide involved in antigen presentation, a polypeptide involved in trafficking and/or migration, or a polypeptide involved in dendritic cell targeting, or a functional fragment or variant thereof. In an embodiment, the T cell priming costimulatory molecule is selected from the group consisting of CD70, CD83, CD80, CD86, CD40, CD154, CD137L (4-1BBL), CD252 (OX40L), CD275 (ICOS-L), CD54 (ICAM-1), CD49a, CD43, CD48, CD112 (PVRL2), CD150 (SLAM), CD155 (PVR), CD265 (RANK), CD270 (HVEM), TL1A, CD127, IL-4R, GITR-L, CD160, CD258, TIM-4, CD153 (CD30L), CD200R (OX2R), CD44, ligands thereof, and functional fragments and variants thereof. In an embodiment, the soluble cytokine is selected from the group consisting of: IL-2, IL-12, IL-6, IL-7, IL-15, IL-18, IL-21, GM-CSF, IL-18, IL-21, IL-27, and functional fragments and variants thereof. In an embodiment, the polypeptide involved in antigen presentation is selected from the group consisting of CD64, MHC I, MHC II, and functional fragments and variants thereof. In an embodiment, the polypeptide involved in trafficking and/or migration is selected from the group consisting of CD183, CCR2, CCR6, CD50, CD197, CD58, CD62L, and functional fragments and variants thereof. In an embodiment, the polypeptide involved in DC targeting is selected from the group consisting of TLR ligands, anti-DEC-205 antibody, an anti-DC-SIGN antibody, and functional fragments and variants thereof.

In some embodiments, the T cell priming polypeptide comprises an amino acid sequence of interleukin 2 (IL-2) (e.g., GenBank Acc. No. AAB46833.1), or a nucleic acid sequence of IL-2 (e.g., GenBank Acc. No. S82692.1). In some embodiments, the T cell priming polypeptide comprises an amino acid sequence of interleukin 12 (IL-12) (e.g., GenBank Acc. No. AAD16432.1), or a nucleic acid sequence of IL-12 (e.g., GenBank Acc. No. AF101062.1). In some embodiments, the T cell priming polypeptide comprises an amino acid sequence of interleukin 6 (IL-6) (e.g., GenBank Acc. No. AAD13886.1 or NP_000591.1), or a nucleic acid sequence of IL-6 (e.g., GenBank Acc. No. 556892.1 or NM_000600.3). In some embodiments, the T cell priming polypeptide comprises an amino acid sequence of interleukin 7 (IL-7) (e.g., GenBank Acc. No. AAH47698.1 or NP_000871.1), or a nucleic acid sequence of IL-7 (e.g., GenBank Acc. No. BC047698.1 or NM_000880.3). In some embodiments, the T cell priming polypeptide comprises an amino acid sequence of interleukin 15 (IL-15) (e.g., GenBank Acc. No. AAU21241.1), or a nucleic acid sequence of IL-15 (e.g., GenBank Acc. No. AY720442.1). In some embodiments, the T cell priming polypeptide comprises an amino acid sequence of interleukin 18 (IL-18) (e.g., GenBank Acc. No. AAK95950.1), or a nucleic acid sequence of IL-18 (e.g., GenBank Acc. No. AY044641.1). In some embodiments, the T cell priming polypeptide comprises an amino acid sequence of interleukin 21 (IL-21) (e.g., GenBank Acc. No. AAG29348.1), or a nucleic acid sequence of IL-21 (e.g., GenBank Acc. No. AF254069.1). In some embodiments, the T cell priming polypeptide comprises an amino acid sequence of GM-CSF (e.g., GenBank Acc. No. AAA52578.1), or a nucleic acid sequence of GM-CSF (e.g., GenBank Acc. No. Ml 1220.1). In some embodiments, the T cell priming polypeptide is an IL-18.

In some embodiments, the expression of the CAR or CARs does not substantially affect the level of expression of the T cell priming polypeptide in the armored CAR T cell. In some embodiments, the CAR comprises an antigen binding domain that binds the antigen, and the expression of the T cell priming polypeptide does not substantially affect the level of expression or cell-killing function of the CAR or CARs in the armored CART cell.

In some embodiments, the lentiviral vector or retroviral vector disclosed herein comprises and delivers more than one T cell priming polypeptides. In an embodiment, the lentiviral vector or retroviral vector comprises 2, 3, 4, 5, 6 or more nucleic acids encoding one or more T cell priming polypeptides; and further comprises a nucleic acid sequence encoding a CAR. In some embodiments, the co-delivery of one or more T cell priming polypeptides does not affect (e.g., substantially decrease or substantially inhibit), the expression or activity of the co-expressed CAR in the armored CAR T cell or armored CAR-expressing immune cell. In some embodiments, the CAR does not affect (e.g., substantially decrease or substantially inhibit), the expression or activity of the co-expressed T cell priming polypeptide.

### V. Car T Cells

One aspect of the present disclosure provides a modified cell, a modified immune cell, a modified CD4⁺ and CD8⁺ cell, or a modified eukaryotic donor cell engineered by the methods described herein.

Another aspect of the present disclosure provides a population of modified cells, a population of modified immune cells, a population of modified CD4⁺ and CD8⁺ cells, or a population modified eukaryotic donor cells engineered by the methods described herein.

Yet another aspect of the present disclosure provides a modified cell, a modified immune cell, a modified CD4⁺ and CD8⁺ cell, or a modified eukaryotic donor cell comprising a lentiviral vector as described herein.

Another aspect of the present disclosure provides a population of modified cells, a population of modified immune cells, a population of modified CD4⁺ and CD8⁺ cells or a population modified eukaryotic donor cells comprising a lentiviral vector described herein. In some embodiments, the modified CD4⁺ and CD8⁺ cell, or the modified eukaryotic donor cell engineered described herein is for use in the production of a protein of interest. In some embodiments of the modified CD4⁺ and CD8⁺ cell, or the modified eukaryotic donor cell engineered described herein, the protein of interest may be selected from the group consisting of an industrial protein, or a therapeutic protein. In some embodiments, the protein of interest may be selected from the group consisting of enzymes, regulatory proteins, receptors, peptides, peptide hormones, cytokines, membrane or transport proteins, vaccine antigens, antigen-binding proteins, immune stimulatory proteins, allergens, full-length antibodies or antibody fragments or derivatives; single chain antibodies, (scFv), Fab fragments, Fy fragments, single domain antibodies (VH or VL fragment), domain antibodies, camelid single domain antibodies (VHH), nanobodies and a combination thereof.

### VI. Compositions

One aspect of the present disclosure provides a composition comprising a modified cell, modified lymphocyte, a modified immune cell, a modified CD4⁺ and CD8⁺ cell, or a modified eukaryotic donor cell produced by the methods described herein. Another aspect of the present disclosure provides a composition comprising a population of modified lymphocytes, a population of modified cells, a population of modified immune cells, a population of modified CD4⁺ and CD8⁺ cells, or a population modified eukaryotic donor cells generated by the methods described herein. Another aspect of the present disclosure provides a composition comprising a lentiviral vector described herein. In some embodiments, the composition further comprises one or more pharmaceutically or physiologically acceptable carriers, diluents, or excipients.

In some embodiments, the compositions described herein are used in medicaments for use in treating a disease or a described herein (e.g., cancer, any malignancy, autoimmune diseases involving cells or tissues which express a tumor antigen as described herein). In some embodiments, the compositions described herein is used in methods for treating, treating a disease or a described herein (e.g., cancer, any malignancy, autoimmune diseases involving cells or tissues which express a tumor antigen as described herein). In some embodiments, provided herein are pharmaceutical compositions comprising a CAR-expressing cell, for example, a plurality of CAR-expressing cells, made by a manufacturing process described herein (for example, the cytokine process, or the activation process described herein).

### VII. Method of Treatment

In one aspect, the present disclosure provides a method for adoptive cell transfer therapy comprising administering to a subject in need thereof a modified immune cell engineered by the methods described herein. In some embodiments, disclosed herein is a method of treating a disease or a condition in a subject, which comprises administering to the subject a population of modified T cells described herein, e.g., a population of modified unstimulated T cells or a population of modified stimulated T cells described herein. In some embodiments, the invention includes a method of treating a disease or condition in a subject comprising administering to a subject in need thereof a composition comprising the modified immune cells described herein.

One aspect of the present disclosure provides a method of treating a disease or condition in a subject, the method comprising administering to the subject in need thereof a therapeutically effective amount of the modified cell, the modified immune cell, the modified CD4⁺ and CD8⁺ cell, or the modified eukaryotic donor cell described herein, thereby treating the disease or condition in the subject. The method of treating a disease or condition in a subject may also comprise administering to the subject in need thereof a therapeutically effective amount of the population of modified cells, the population of modified immune cells, the population of modified CD4⁺ and CD8⁺ cells, or the population of modified eukaryotic donor cells made by the methods described herein. The method of treating a disease or condition in a subject may also comprise administering to the subject in need thereof a therapeutically effective amount of the composition described herein.

In some embodiments, the modified immune cell, the modified CD4⁺ and CD8⁺ cell, or the modified eukaryotic donor cell is autologous to the subject. In some embodiments, the modified immune cell, the modified CD4⁺ and CD8⁺ cell, or the modified eukaryotic donor cell is allogeneic to the subject; In some embodiments, the modified immune cell, the modified CD4⁺ and CD8⁺ cell, or the modified eukaryotic donor cell is a xenogeneic to the subject.

In some embodiments, the modified cell, the modified immune cell, the modified CD4⁺ and CD8⁺ cell, or the modified eukaryotic donor cell is allogeneic to the subject. In some embodiments, the subject is a human.

A. Diseases and Conditions

One aspect of the present disclosure provides an adoptive cell transfer therapy method for a disease or condition. In some embodiments, the disease or condition may be selected from the group consisting of cancer, an autoimmune disease, Lupus, a neurodegenerative disease or condition, Alzheimer's disease, multiple sclerosis, an infectious disease, a fibrotic condition, liver fibrosis, lung fibrosis, post-ischemic fibrosis, a genetic disorder, sickle cell anemia, hemophilia, and/or beta-thalassemia. In some embodiments, the disease or condition selected from a cancer, any malignancy, autoimmune diseases involving cells or tissues which express a tumor antigen as described herein.

In some embodiments, the disease or condition is selected from the group consisting of viral infection, a bacterial infection, a parasitic infections, a cancer, a malignancy, a non-cancerous condition, an autoimmune disease, a fibrotic disease, Alzheimer's disease, protein deficiency conditions, and factor VIII deficiency.

The disease may be a cancer selected from the group consisting of blood cancers, lymphoma, leukemia, multiple myeloma, diffuse large B-cell lymphoma, follicular lymphoma, mantle cell lymphoma, B-cell acute lymphoblastic leukemia(ALL), pre-B ALL, breast cancer, triple-negative breast cancer, prostate cancer, ovarian cancer, glioma, glioblastoma, renal cell carcinoma, kidney cancer, mesothelioma, cervical cancer, skin cancer, pancreatic cancer, colorectal cancer, renal cancer, liver cancer, lung cancer, lung adenocarcinoma, gallbladder cancer, colon cancer, cervical squamous cell carcinoma, non-small cell lung cancer, small cell lung cancer, Merkel cell carcinoma, hepatocellular carcinoma, esophagus cancer, brain cancer, melanoma, Hodgkin's lymphoma, non-Hodgkin's lymphoma, urothelial carcinoma, gastric cancer, and any combination thereof.

In some embodiments, the cancer is a solid cancer selected from the group consisting of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof.

In some embodiments, the cancer is a liquid cancer, for example, chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitf s lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

B. Payload

In some embodiments, the transfected cells comprises a CAR, an engineered TCR, a KIR, an antigen-binding polypeptide, a cell surface receptor ligand, a tumor antigen, a switch receptor, a dominant negative receptor, and/or a polypeptide that enhances immune function (e.g., T cell priming or T cell infiltration). In some embodiments, a polypeptide that enhances the immune cell function, or a functional derivative thereof is selected from a chemokine, a chemokine receptor, a cytokine, a cytokine receptor, Interleukin-7 (IL-7), Interleukin-7 receptor (IL-7R), Interleukin-15 (IL-15), Interleukin-15 receptor (IL-15R), Interleukin-21 (IL-21), Interleukin-18 (IL-18), Interleukin-18 receptor (IL-18R), CCL21, CCL19, or a combination thereof. In some embodiments, a chemokine, a chemokine receptor, a cytokine, a cytokine receptor, IL-7, IL-7R, IL-15, IL-15R, IL-21, IL-18, IL-18R, C-C Motif Chemokine Ligand 21 (CCL21), or C-C Motif Chemokine Ligand 19 (CCL19) is an immune function-enhancing factor that improves the fitness of the claimed modified immune cell. Without wishing to be bound by theory, the addition of a nucleic acid encoding a chemokine, a chemokine receptor, a cytokine, a cytokine receptor, IL-7, IL-7R, IL-15, IL-15R, IL-21, IL-18, IL-18R, CCL21, or CCL19 to the modified immune cell of the present disclosure enhances the immunity-inducing effect and antitumor activity of the modified immune cell.

In some embodiments, the switch receptor is selected from the group consisting of PD-1-CD28, PD-1A132L-CD28, PD-1-CD27, PD-1A132L-CD27, PD-1-4-1BB, PD-1A132L-4-1BB, PD-1-ICOS, PD-1A132L-ICOS, PD-1-IL12Rβ1, PD-1A132L-IL12Rβ1, PD-1-IL12Rβ2, PD-1A132L-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2.

In some embodiments, the dominant negative receptor is PD-1, CTLA4, BTLA, TGFβRII, VSIG3, VSIG8, or TIM-3 dominant negative receptor.

In some embodiments, a polypeptide that enhances immune function is selected from the group consisting of a chemokine, a chemokine receptor, a cytokine, a cytokine receptor, IL-7, IL-7R, IL-15, IL-15R, IL-21, IL-18, IL-18R, CCL21, CCL19, or a combination thereof.

C. Combination Therapy

In some embodiments, the method of treating a disease further comprises administering to the subject an additional therapeutic agent or an additional therapy. In some cases, an additional therapeutic agent disclosed herein comprises a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone. In some cases, the additional therapeutic agent comprises a first-line therapy. As used herein, “first-line therapy” comprises a primary treatment for a subject with a cancer. In some instances, the cancer is a primary cancer. In other instances, the cancer is a metastatic or recurrent cancer. In some cases, the first-line therapy comprises chemotherapy. In other cases, the first-line treatment comprises radiation therapy. A skilled artisan would readily understand that different first-line treatments may be applicable to different type of cancers. In some cases, the additional therapeutic agent comprises an immune checkpoint inhibitor. In some instances, the immune checkpoint inhibitor comprises an inhibitors such as an antibody or fragments (e.g., a monoclonal antibody, a human, humanized, or chimeric antibody) thereof, RNAi molecules, or small molecules to PD-1, PD-L1, CTLA4, PD-L2, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. Exemplary checkpoint inhibitors include pembrolizumab, nivolumab, tremelimumab, or ipilimumab. In some embodiments, the additional therapy comprises radiation therapy.

In some embodiments, the additional therapy comprises surgery.

### VIII. Kits

One aspect of the present disclosure provides a kit comprising a population of modified immune cells or a population of modified CD4⁺ and CD8⁺ cells, or a population of engineered by the methods described herein. Another aspect of the present disclosure provides a kit comprising a lentiviral vector described herein.

### IX. Definitions

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein. It is also to be understood that the terminology used herein is for the purpose of describing particular embodiments only, and is not intended to be limiting.

The practice of the present disclosure will employ, unless otherwise indicated, conventional techniques of tissue culture, immunology, molecular biology, microbiology, cell biology and recombinant DNA, which are within the skill of the art. See e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); Lundblad and Macdonald eds. (2010) Handbook of Biochemistry and Molecular Biology, 4th edition; and Herzenberg et al. eds (1996) Weir's Handbook of Experimental Immunology, 5th edition.

As used herein, the singular forms “a”, “an,” and “the” include plural referents unless the context clearly indicates otherwise. For example, the term “a cell” includes a plurality of cells, including mixtures thereof, and means one cell or more than one cell

As used herein, the term “About” refers to a value includes the standard deviation of error for the device or method being employed to determine the value. The term “about” when used before a numerical designation, e.g., temperature, time, amount, and concentration, including range, indicates approximations which may vary by (+) or (—) (±) 20%, 15%, 10%, 5%, 3%, 2%, or 1%. Preferably ±5%, more preferably ±1%, and still more preferably ±0.1% from the specified value, as such variations are appropriate to perform the disclosed methods.

As used herein, the term “Activation” refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, the term “Allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

As used herein, the term “Analogue”, in relation to polypeptides or polynucleotides includes any mimetic, that is, a chemical compound that possesses at least one of the endogenous functions of the polypeptides or polynucleotides which it mimics. Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins used in the present disclosure may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine. Conservative substitutions may be made.

As used herein, the term “Antibody” refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies (scFv) and humanized antibodies. In some embodiments, antibody refers to such assemblies (e.g., intact antibody molecules, immunoadhesins, or variants thereof) which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

The term “Antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

As used herein, the term “Antibody heavy chain,” as used herein, refers to the larger of the two types of polypeptide chains present in all antibody molecules in their naturally occurring conformations.

As used herein, an “Antibody light chain,” refers to the smaller of the two types of polypeptide chains present in all antibody molecules in their naturally occurring conformations. α and β light chains refer to the two major antibody light chain isotypes. The antigen binding domain of (e.g., a chimeric antigen receptor) includes antibody variants. As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bi-specific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

As used herein, the term “Antigen” or “Ag” is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit a desired immune response. Moreover, the skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “Anti-tumor effect” refers to a biological effect which can be manifested by a decrease in tumor volume, a decrease in the number of tumor cells, a decrease in the number of metastases, an increase in life expectancy, or amelioration of various physiological symptoms associated with the cancerous condition. In some embodiments, an “anti-tumor effect” can also be manifested by the ability of the peptides, polynucleotides, cells and antibodies of the invention in prevention of the occurrence of tumor in the first place.

As used herein, the term “Apheresis” refers to an automated blood collection system in which whole blood is separated into its component, such that a blood donor only gives those components of the blood needed for patients. In some embodiments, the extracorporeal apheresis process is a recognized process by which the blood of a donor or patient is removed from the donor or patient and passed through an apparatus that separates out selected particular constituent(s) and returns the remainder to the circulation of the donor or patient, for example, by retransfusion-using a single needle. Thus, in the context of “an apheresis sample” refers to a sample obtained using apheresis.

In some embodiments, the apheresis device or system is a standard apheresis device selected from the group consisting of COBE® Spectra, Spectra Optia, TRIMA®, and SPECTRA OPTIA® systems (all marketed by Gambro BCT), Fenwal AMICUS®, AMICUS™ and CS-3000⁺™ or any equivalent. In some embodiments, the separation device is AMICUS®. See e.g., U.S. Pat. No. 6,027,657.

In some embodiments, the apheresis device o is a multifunctional automated apheresis device. In some embodiments, the separation device may be a multifunctional automated apparatus used in a hospital or clinical facility to perform various collection protocols, including, but not limited to the collection of blood and other blood components such as platelets, plasma, red blood cells, and granulocytes and/or the performance of plasma/RBC exchange

As used herein, the term “Auto-antigen” means, in accordance with the present invention, any self-antigen, which is recognized by the immune system as being foreign. In some embodiments, Auto-antigens comprise, but are not limited to, cellular proteins, phosphoproteins, cellular surface proteins, cellular lipids, nucleic acids, glycoproteins, including cell surface receptors.

As used herein, the term “Autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, cancer, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “Autologous” is meant to refer to any material derived from the same individual into whom the material may later be re-introduced.

As used herein, the term “Cas,” “Cas molecule,” or “Cas molecule” refers to an enzyme from bacterial Type II CRISPR/Cas system responsible for DNA cleavage. Cas includes wild-type protein as well as functional and non-functional mutants thereof.

As used herein, the term “Cancer” refers to a disease characterized by the rapid and uncontrolled growth of aberrant cells. Cancer cells can spread locally or through the bloodstream and lymphatic system to other parts of the body. As used herein, the term “cancer” refers to a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, metastatic castrate-resistant prostate cancer, melanoma, synovial sarcoma, advanced TnMuc1 positive solid tumors, neuroblastoma, neuroendocrine tumors, and the like. In certain embodiments, the cancer is medullary thyroid carcinoma. In certain embodiments, the cancer is prostate cancer. In certain embodiments, the cancer is mesothelioma or a mesothelin expressing cancer. In some embodiments, the cancer is metastatic castrate-resistant prostate cancer. The terms “cancer” and “tumor” are used interchangeably herein, and both terms encompass solid and liquid tumors, diffuse or circulating tumors. In some embodiments, the cancer or tumor includes premalignant, as well as malignant cancers and tumors.

As used herein, the term “Cancer associated antigen” or “Tumor antigen” interchangeably refers to a molecule (typically a protein, carbohydrate or lipid) that is expressed on the surface of a cancer cell, either entirely or as a fragment (e.g., WIC/peptide), and which is useful for the preferential targeting of a pharmacological agent to the cancer cell. In some embodiments, a tumor antigen is a marker expressed by both normal cells and cancer cells (e.g., a lineage marker such as CD19 on B cells). In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., WIC/peptide), and not synthesized or expressed on the surface of a normal cell. In some embodiments, the CARs of the present invention includes CARs comprising an antigen binding domain (e.g., antibody or antibody fragment) that binds to a WIC presented peptide. Normally, peptides derived from endogenous proteins fill the pockets of Major histocompatibility complex (MHC) class I molecules, and are recognized by T cell receptors (TCRs) on CD8⁺ T lymphocytes. The MEW class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described. For example, TCR-like antibody can be identified from screening a library, such as a human scFv phage displayed library.

As used herein, the term “Cancer-supporting antigen” or “tumor-supporting antigen” interchangeably refers to a molecule (typically a protein, carbohydrate or lipid) that is expressed on the surface of a cell that is, itself, not cancerous, but supports the cancer cells by promoting their growth or survival (e.g., resistance to immune cells). Exemplary cells of this type include stromal cells and myeloid-derived suppressor cells (MDSCs). The tumor-supporting antigen itself need not play a role in supporting the tumor cells so long as the antigen is present on a cell that supports cancer cells.

As used herein, the terms “Cell,” “Cell line,” and “Cell culture” may be used interchangeably. In some embodiments, the host cell is an immune cell or precursor thereof. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In some embodiments, the host cell is a T cell, an NK cell, or an NKT cell. In some embodiments, the immune cell is selected from the group consisting of a T cell, a natural killer cell (NK cell), a natural killer T cell, a lymphoid progenitor cell, a hematopoietic stem cell, a stem cell, a macrophage, and a dendritic cell. In some embodiments, the immune cell is a CD4⁺ T cell or a CD8⁺ T cell. In some embodiments, the immune cell is an allogeneic T cell or autologous T cell. In some embodiments, the allogeneic T cell or autologous T cell is human.

As used herein, a “Cell-surface marker” refers to any molecule that is expressed on the surface of a cell. Cell-surface expression usually requires that a molecule possesses a transmembrane domain. Many naturally occurring cell-surface markers are termed “CD” or “cluster of differentiation” molecules. Cell-surface markers often provide antigenic determinants to which antibodies can bind.

As used herein, the term “Central memory T cells” refers to a subset of T cells that in humans are CD45RO positive and express CCR7. In some embodiments, central memory T cells express CD95. In some embodiments, central memory T cells express IL-2R, IL-7R and/or IL-15R. In some embodiments, central memory T cells express CD45RO, CD95, IL-2 receptor b, CCR7, and CD62L. In some embodiments, surface expression levels of markers are assessed using flow cytometry. The term “stem memory T cells,” “stem cell memory T cells,” “stem cell-like memory T cells,” “memory stem T cells,” “T memory stem cells,” “T stem cell memory cells” or “TSCM cells” refers to a subset of memory T cells with stem cell-like ability, for example, the ability to self-renew and/or the multipotent capacity to reconstitute memory and/or effector T cell subsets. In some embodiments, stem memory T cells express CD45RA, CD95, IL-2 receptor b, CCR7, and CD62L. In some embodiments, surface expression levels of markers are assessed using flow cytometry. In some embodiments, exemplary stem memory T cells are disclosed in Gattinoni et al., Nat Med. 23(1): 18-27 (2017).

For clarity purposes, unless otherwise noted, classifying a cell or a population of cells as “not expressing,” or having an “absence of” or being “negative for” a particular marker may not necessarily mean an absolute absence of the marker. The skilled artisan can readily compare the cell against a positive and/or a negative control, and/or set a predetermined threshold, and classify the cell or population of cells as not expressing or being negative for the marker when the cell has an expression level below the predetermined threshold or a population of cells has an overall expression level below the predetermined threshold using conventional detection methods, e.g., using flow cytometry,

As used herein, the term “Chimeric antigen receptor” or “CAR,” refers to an artificial T cell receptor that is engineered to be expressed on an immune effector cell or precursor cell thereof and specifically bind an antigen. CARs may be used in adoptive cell therapy with adoptive cell transfer. In some embodiments, adoptive cell transfer (or therapy) comprises removal of T cells from a patient, and modifying the T cells to express the receptors specific to a particular antigen. In some embodiments, the CAR has specificity to a selected target, for example, ROR1, mesothelin, c-Met, PSMA, PSCA, Folate receptor alpha, Folate receptor beta, EGFR, EGFRvIII, GPC2, GPC2, Mucin 1(MUC1), Tn antigen ((Tn Ag) or (GalNAca-Ser/Thr)), TnMUC1, GDNF family receptor alpha-4 (GFRa4), fibroblast activation protein (FAP), or Interleukin-13 receptor subunit alpha-2 (IL-13Ra2 or CD213A2). In some embodiments, CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target cancers by redirecting the specificity of a T cell expressing the CAR specific for tumor associated antigens.

As used herein, the term “Conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

As used herein, the term “Co-stimulatory ligand,” includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MEW molecule loaded with peptide, mediates a T cell response, including, but not limited to, proliferation, activation, differentiation, and the like. A co-stimulatory ligand can include, but is not limited to, CD2, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

As used herein, a “Co-stimulatory molecule” refers to the cognate binding partner on a T cell that specifically binds with a co-stimulatory ligand, thereby mediating a co-stimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA, a Toll ligand receptor, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR). In some embodiments, a co-stimulatory molecule includes OX40, CD27, CD2, CD28, ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

As used herein, the term “Co-stimulatory signal” refers to a signal, which in combination with a primary signal, such as TCR/CD3 ligation, leads to T cell proliferation and/or upregulation or downregulation of key molecules. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that specifically binds with CD83, and the like.

As used herein, the term “CRISPR” refers to clustered regularly interspaced short palindromic repeats system. The term “CRISPR system,” “CRISPR/Cas,” “CRISPR/Cas system,” or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via RNA-guided DNA cleavage. In the type II CRISPR/Cas system, short segments of foreign DNA, termed “spacers” are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

In some embodiments, the term “CRISPR system,” “CRISPR/Cas,” “CRISPR/Cas system,” or “CRISPR” refers to a set of molecules comprising an RNA-guided nuclease or other effector molecule and a gRNA molecule that together are necessary and sufficient to direct and effect modification of nucleic acid at a target sequence by the RNA-guided nuclease or other effector molecule. In some embodiments, a CRISPR system comprises a gRNA and a Cas protein, e.g., a Cas3, Cas4, Cas8a, Cas8b, Cas9, Cas10, Cas10d, Cas12a, Cas12b, Cas12d, Cas12e, Cas 12f, Cas12g, Cas12h, Cas12i, Cas13, Cas14, CasX, Cse1, Csy1, Csn2, Cpf1, C2c1, Csm2, Cmr5, Fok1, S. pyogenes Cas9 (spCas9), or Staphylococcus aureus Cas9 (saCas9) protein. Such systems comprising a Cas or modified Cas molecule are referred to herein as “Cas systems” or “CRISPR/Cas systems.” In some embodiments, the gRNA molecule and Cas molecule may be complexed, to form a ribonuclear protein (RNP) complex.

To direct Cas9 to cleave sequences of interest, crRNA-tracrRNA fusion transcripts, hereafter referred to as “guide RNAs” or “gRNAs” may be designed, from human U6 polymerase III promoter. CRISPR/CAS mediated genome editing and regulation, highlighted its transformative potential for basic science, cellular engineering and therapeutics.

As used herein, the term “crRNA” as the term is used in connection with a gRNA molecule, is a portion of the gRNA molecule that comprises a targeting domain and a region that interacts with a tracr to form a flagpole region.

As used herein, the term “Cytokine” (e.g., IL-2, IL-7, IL-15, IL-18 IL-21, or IL-6) includes full length, a fragment, or a variant. A functional variant of a naturally-occurring cytokine may include fragments and functional variants thereof having at least 10%, 30%, 50%, or 80% of the activity (e.g., the immunomodulatory activity, of the naturally-occurring cytokine). In some embodiments, the cytokine has an amino acid sequence that is substantially identical (e.g., at least about 85%, 90%, 91%, 92%, 93%, 94%, 95%, 96%, 97%, 98% or 99% identity) to a naturally-occurring cytokine, or is encoded by a nucleotide sequence that is substantially identical (e.g., at least about 75%, 80%, 85%, 90%, 91%, 92%, 93%, 94%, 95%, 96%, 97%, 98% or 99% identity) to a naturally-occurring nucleotide sequence encoding a cytokine. In some embodiments, as understood in context, the cytokine further comprises a receptor domain, e.g., a cytokine receptor domain (e.g., an IL-15/IL-15R; IL-18/IL-18R).

As used herein, the term “Derived from” refers to a relationship between a first and a second molecule. It generally refers to structural similarity between the first molecule and a second molecule and does not include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not include a limitation to a particular process of producing the intracellular signaling domain, for example, it does not mean that, to provide the intracellular signaling domain, one must start with a CD3zeta sequence and delete unwanted sequence, or impose mutations, to arrive at the intracellular signaling domain.

As used herein, the term “Disease” refers to a state of health of an animal wherein the animal cannot maintain homeostasis, and wherein if the disease is not ameliorated then the animal's health continues to deteriorate. In contrast, the term “disorder” in an animal refers to a state of health in which the animal is able to maintain homeostasis, but in which the animal's state of health is less favorable than it would be in the absence of the disorder. Left untreated, a disorder does not necessarily cause a further decrease in the animal's state of health.

As used herein, “Disease associated with expression of a tumor antigen” includes, but is not limited to, a disease associated with expression of a tumor antigen or condition associated with cells which express a tumor antigen including, but not limited to proliferative diseases such as a cancer or malignancy or a precancerous condition such as a myelodysplasia, a myelodysplastic syndrome or a preleukemia; or a noncancer related indication associated with cells, which express a tumor antigen. In some embodiments, a cancer associated with expression of a tumor antigen is a hematological cancer. In some embodiments, a cancer associated with expression of a tumor antigen is a solid cancer. Further diseases associated with expression of a tumor antigen include, but not limited to, atypical and/or non-classical cancers, malignancies, precancerous conditions or proliferative diseases associated with expression of a tumor antigen. Non-cancer related indications associated with expression of a tumor antigen include, but are not limited to, autoimmune disease, (e.g., lupus), inflammatory disorders (allergy and asthma) and transplantation. In some embodiments, the tumor antigen-expressing cells express, or at any time expressed, mRNA encoding the tumor antigen. In some embodiments, the tumor antigen-expressing cells produce the tumor antigen protein (e.g., wild-type or mutant), and the tumor antigen protein may be present at normal levels or reduced levels. In some embodiment, the tumor antigen-expressing cells produced detectable levels of a tumor antigen protein at one point, and subsequently produced substantially no detectable tumor antigen protein.

As used herein, the term “Downregulation” refers to the decrease or elimination of gene expression of one or more genes.

As used herein, the term “Encoding” refers to the inherent property of specific sequences of nucleotides in a polynucleotide (such as a gene, a cDNA, or an mRNA), to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

As used herein, the terms “Effective amount” and “Therapeutically effective amount” are used interchangeably herein, refer to an amount of a compound, formulation, material, pharmaceutical agent, or composition, as described herein effective to achieve a desired physiological, therapeutic, or prophylactic outcome in a subject in need thereof. Such results may include, but are not limited to an amount that when administered to a mammal, causes a detectable level of immune response compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like. The effective amount may vary among subjects depending on the health and physical condition of the subject to be treated, the taxonomic group of the subjects to be treated, the formulation of the composition, assessment of the subject's medical condition, and other relevant factors.

As used herein, the term “Env” shall mean an endogenous lentiviral envelope or a heterologous envelope, as described herein.

As used herein, the term “Enriching” and “Enriched” are used interchangeably to mean that the yield (fraction) of cells (e.g., T cells), for use in the manufacturing process described herein, is increased by at least about 5 fold, at least about 10-fold, at least about 20-fold, at least about 30-fold, at least about 40-fold, at least about 50-fold, at least about 60-fold, at least about 70-fold, at least about 80-fold, at least about 90-fold, at least about 100-fold or more over the fraction of enriched apheresis product.

As used herein, the term “Endogenous” refers to any material from or produced inside an organism, cell, tissue or system.

As used herein, the term “Expand” as used herein refers to increasing in number, as in an increase in the number of immune cells (e.g. T cells). In some embodiments, the immune cells (e.g. T cells) that are expanded ex vivo increase in number relative to the number originally present in the culture. In another embodiment, the immune cells (e.g. T cells) that are expanded ex vivo increase in number relative to other cell types in the culture.

As used herein, the term “Expression” refers to the transcription and/or translation of a particular nucleotide sequence driven by a promoter.

As used herein, the term “Exogenous” refers to any material introduced from or produced outside an organism, cell, tissue or system.

As used herein, the term “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “Extended packaging signal” or “Extended packaging sequence” refers to the use of sequences around the psi sequence with further extension into the gag gene. The inclusion of these additional packaging sequences may increase the efficiency of insertion of vector RNA into viral particles. As an example, for the Murine Leukemia Virus (MoMLV) the minimum core packaging signal is encoded by the sequence (counting from the 5′ LTR cap site) from approximately nucleotide 144, up through the Pst I site (nucleotide 567). The extended packaging signal of MoMLV includes the sequence beyond nucleotide 567 up through the start of the gag/pol gene (nucleotide 621), and beyond nucleotide 1040 (Bender et al. (1987)). These sequences include about a third of the gag gene sequence.

As used herein, the term “ex vivo,” refers to cells that have been removed from a living organism, (e.g., a human) and propagated outside the organism (e.g., in a culture dish, test tube, or bioreactor).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

As used herein, the term “Flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3. n=4, n=5 and n=6, n=7, n=8, n=9 and n=10 (SEQ ID NO:6592). In one embodiment, the flexible polypeptide linkers include, but are not limited to, (Gly4 Ser)4 or (Gly4 Ser)3. In another embodiment, the linkers include multiple repeats of (Gly2Ser), (GlySer) or (Gly3Ser).

As used herein, a “Fragment” is also a variant and the term typically refers to a selected region of a polypeptide or polynucleotide that is of interest either functionally or, for example, in an assay. “Fragment” thus refers to an amino acid or nucleic acid sequence that is a portion of a full-length polypeptide or polynucleotide.

As used herein, “Functional variant” refers to a polypeptide that has a substantially identical amino acid sequence to a reference amino acid sequence, or is encoded by a substantially identical nucleotide sequence, and is capable of having one or more activities of the reference amino acid sequence.

As used herein, the term “Guide RNA,” “Guide RNA molecule,” “gRNA molecule” or “gRNA” are used interchangeably, and refers to a set of nucleic acid molecules that promote the specific directing of a RNA-guided nuclease or other effector molecule (typically in complex with the gRNA molecule) to a target sequence. In some embodiments, said directing is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In some embodiments, a gRNA molecule consists of a single contiguous polynucleotide molecule, referred to herein as a “single guide RNA” or “sgRNA” and the like. In some embodiments, a gRNA molecule consists of a plurality, usually two, polynucleotide molecules, which are themselves capable of association, usually through hybridization, referred to herein as a “dual guide RNA” or “dgRNA,” and the like. gRNA molecules are described in more detail below, but generally include a targeting domain and a tracr. In some embodiments the targeting domain and tracr are disposed on a single polynucleotide. In other embodiments, the targeting domain and tracr are disposed on separate polynucleotides.

As used herein, the term “Host cell” includes cells transfected, infected, or transduced in vivo, ex vivo, or in vitro with a recombinant vector or a polynucleotide of the invention. Host cells may include packaging cells, producer cells, and cells infected with viral vectors. In some embodiments, host cells infected with the lentiviral vector of the disclosure are administered to a subject in need of therapy. In some embodiments, the term “target cell” is used interchangeably with host cell and refers to transfected, infected, or transduced cells of a desired cell type. In preferred embodiments, the target cell is a T cell.

As used herein, the term “Homologous” refers to the subunit sequence identity between two polymeric molecules (e.g., between two nucleic acid molecules, such as, two DNA molecules or two RNA molecules), or between two polypeptide molecules. When a subunit position in both of the two molecules is occupied by the same monomeric subunit, then they are homologous at that position. For example, if a position in each of two DNA molecules is occupied by adenine, then the two DNA molecules are homologous. The homology between two sequences is a direct function of the number of matching or homologous positions. For example, if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

As used herein, the term “Homologue” means an entity having a certain homology with the wild type amino acid sequence and the wild type nucleotide sequence. The term “homology” can be equated with “identity”. In the present context, a homologous sequence is taken to include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

A homologous sequence is taken to include a nucleotide sequence which may be at least 50%, 55%, 65%, 75%, 85% or 90% identical, preferably at least 95% or 97% or 99% identical to the subject sequence. Although homology can also be considered in terms of similarity, in the context of the present disclosure it is preferred to express homology in terms of sequence identity. Homology comparisons can be conducted by eye, or more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalizing unduly the overall homology score. This is achieved by inserting “gaps” in the sequence alignment to try to maximize local homology.

However, these more complex methods assign “Gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

j Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Research 12:387). Examples of other software that can perform sequence comparisons include, but are not limited to, the BLAST package, FASTA and the GENEWORKS suite of comparison tools. Both BLAST and FASTA are available for offline and online searching. However, for some applications, it is preferred to use the GCG Bestfit program. Another tool, called BLAST 2 Sequences is also available for comparing protein and nucleotide sequences.

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62. Once the software has produced an optimal alignment, it is possible to calculate percentage homology, preferably percentage sequence identity. The software typically does this as part of the sequence comparison and generates a numerical result.

As used herein, the term “Hybrid vector” refers to a vector, LTR or other nucleic acid containing both retroviral sequences (e.g., lentiviral), and non-retroviral sequences (e.g., lentiviral viral sequences). In one embodiment, a hybrid vector refers to a vector or transfer plasmid comprising retroviral (e.g., lentiviral) sequences for reverse transcription, replication, integration and/or packaging.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

As used herein, the term “Identity” refers to the subunit sequence identity between two polymeric molecules particularly between two amino acid molecules, such as, between two polypeptide molecules. When two amino acid sequences have the same residues at the same positions, then they are identical at that position. For example, if a position in each of two polypeptide molecules is occupied by an Arginine, then the two polypeptides are identical. The identity or extent to which two amino acid sequences have the same residues at the same positions in an alignment is often expressed as a percentage. The identity between two amino acid sequences is a direct function of the number of matching or identical positions. For example, if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the term “Immunoglobulin” or “Ig,” defines a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the term “Immune response” as used herein is defined as a cellular response to an antigen that occurs when lymphocytes identify antigenic molecules as foreign and induce the formation of antibodies and/or activate lymphocytes to remove the antigen.

As used herein, the term “Immune effector cell,” refers to a cell that is involved in an immune response, e.g., in the promotion of an immune effector response. Examples of immune effector cells include T cells (e.g., alpha/eta T cells and gamma/delta T cells), B cells, natural killer (NK) cells, natural killer T (NKT) cells, mast cells, and myeloic-derived phagocytes.

As used herein, the term “Immune effector function or immune effector response,” refers to a function or response that enhances or promotes an immune attack of a target cell. In some embodiment, an immune effector function or response refers to a property of a T or NK cell that promotes the killing or the inhibition of growth or proliferation, of a target cell. In the case of a T cell, primary stimulation and co-stimulation are examples of immune effector function or response.

As used herein, the term “Inhibitory molecule” refers to a molecule, which when activated, causes or contributes to an inhibition of cell survival, activation, proliferation and/or function; and the gene encoding said molecule and its associated regulatory elements (e.g., promoters). In some embodiments, an inhibitory molecule is a molecule expressed on an immune effector cell (e.g., on a T cell). Non-limiting examples of inhibitory molecules are PD-1, PD-L1, PD-L2, CTLA4, TIM3, LAG3, CEACAM (e.g., CEACAM-1, CEACAM-3 and/or CEACAM-5), VISTA, TGFβIIR, VSIG3, VSIG 8, BTLA, TIGIT, LAIR1, CD160, 2B4, CD80, CD86, B7-H3 (CD276), B7-H4 (VTCN1), HVEM (TNFRSF14 or CD107), KIR, A2aR, MHC class I, MHC class II, GALS, adenosine, and TGF beta. It will be understood that the term inhibitory molecule refers to the gene (and its associated regulatory elements) encoding an inhibitory molecule protein when it is used in connection with a target sequence or gRNA molecule. In some embodiments, gene encoding the inhibitory molecule is BTLA, PD-1, TIM-3, VSIG3, VSIG8, CTLA4, or TGFβIIR. In some embodiments, the gene encoding the inhibitory molecule is VSIG3. In some embodiments, the gene encoding the inhibitory molecule is PD-1. In some embodiments, the gene encoding the inhibitory molecule is TGFβIIR.

As used herein, the term “Induced pluripotent stem cell” or “iPS cell” refers to a pluripotent stem cell that is generated from adult cells, such immune cells (i.e. T cells). The expression of reprogramming factors, such as Klf4, Oct3/4 and Sox2, in adult cells convert the cells into pluripotent cells capable of propagation and differentiation into multiple cell types.

As used herein, the term “Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, “In vitro transcribed RNA” refers to RNA that has been synthesized in vitro. In some embodiments the RNA is mRNA. Generally, the in vitro transcribed RNA is generated from an in vitro transcription vector. The in vitro transcription vector comprises a template that is used to generate the in vitro transcribed RNA.

As used herein, the term “Knockout” refers to the ablation of gene expression of one or more genes.

As used herein, the term “Lentiviral vector” refers to a viral vector or plasmid containing structural and functional genetic elements, or portions thereof, including LTRs that are primarily derived from a lentivirus. In some embodiments, the terms “Lentiviral vector,” and “Lentiviral expression vector” may be used to refer to lentiviral transfer plasmids and/or infectious lentiviral particles. Where reference is made herein to elements such as cloning sites, promoters, regulatory elements, heterologous nucleic acids, etc. In some embodiments, the sequences of these elements are present in RNA form in the lentiviral particles of the invention and are present in DNA form in the DNA plasmids of the invention.

As used herein, the term “Lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The lentivirus family differs from retroviruses in that lentiviruses have the capability to infect both dividing and non-dividing cells. In contrast, retroviruses, such as MLV, are unable to infect non-dividing or slowly dividing cells such as those that make up, for example, muscle, brain, lung and liver tissue.

A lentiviral or lentivirus vector, as used herein, is a vector which comprises at least one component part derivable from a lentivirus. Preferably, that component part is involved in the biological mechanisms by which the vector infects cells, expresses genes or is replicated. The lentiviral vector may be a “non-primate” vector, i.e., derived from a virus which does not primarily infect primates, especially humans. The non-primate lentivirus may be any member of the family of lentiviridae, which does not naturally infect a primate and may include a feline immunodeficiency virus (FIV), a bovine immunodeficiency virus (BIV), a caprine arthritis encephalitis virus (CAEV), a Maedi visna virus (MVV) or an equine infectious anaemia virus (EIAV).

As used herein, the term “Lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

As used herein, the term “Lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

As used herein, the term “Nanomatrix” refers to a nanostructure comprising a matrix of mobile polymer chains. The nanomatrix is 1 to 500 nm, for example, 10 to 200 nm, in size. In some embodiments, the matrix of mobile polymer chains is attached to one or more agonists which provide activation signals to T cells, for example, agonist anti-CD3 and/or anti-CD28 antibodies. In some embodiments, the nanomatrix comprises a colloidal polymeric nanomatrix attached, for example, covalently attached, to an agonist of one or more stimulatory molecules and/or an agonist of one or more costimulatory molecules. In some embodiments, the agonist of one or more stimulatory molecules is a CD3 agonist (for example, an anti-CD3 agonistic antibody). In some embodiments, the agonist of one or more costimulatory molecules is a CD28 agonist (for example, an anti-CD28 agonistic antibody). In some embodiments, the nanomatrix is characterized by the absence of a solid surface, for example, as the attachment point for the agonists, such as anti-CD3 and/or anti-CD28 antibodies. In some embodiments, the nanomatrix is the nanomatrix disclosed in W02014/048920A1 or as given in the MACS® GMP T Cell Trans Act™ kit from Miltenyi Biotcc GmbH. MACS® GMP T Cell TransAct™ consists of a colloidal polymeric nanomatrix covalently attached to humanized recombinant agonist antibodies against human CD3 and CD28.

As used herein, a “Marker,” a refers to the characteristics and/or phenotype of a cell. Markers can be used for selection of cells comprising characteristics of interest. Markers can vary with specific cells. Markers can be, for example, molecules expressed by or on a given cell type, morphological, functional or biochemical (enzymatic). Preferably, such markers are proteins, and more preferably, proteins that possess an epitope for antibodies or other binding molecules available in the art. Examples of morphological characteristics or traits include, but are not limited to, shape, size, appearance (e.g., smooth, translucent), density, granularity, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages.

As used herein, the term “Minimal viral genome” means that the viral vector has been manipulated so as to remove the non-essential elements and to retain the essential elements in order to provide the required functionality to infect, transduce and deliver a nucleotide sequence of interest to a target host cell. Further details of this strategy can be found in our WO 98/17815. A minimal viral genome of the present disclosure may comprise (5′) R-U5-one or more nucleotide sequence of interest sequences—U3-R (3′).

In one embodiment, the minimal viral genome comprises little to no retroviral or lenti viral sequences. For example, it may only comprise a transgene of interest (e.g., a CAR) and a packaging signal. However, the plasmid vector used to produce the viral genome within a host cell/packaging cell will also include transcriptional regulatory control sequences operably linked to the viral genome to direct transcription of the genome in a host cell/packaging cell. These regulatory sequences may be the natural sequences associated with the transcribed viral sequence, i.e. the 5′ U3 region, or they may be a heterologous promoter such as another viral promoter, for example the constitutive transport element (CMV) promoter.

Some lentiviral genomes require additional sequences for efficient virus production. For example, in the case of HIV, rev and RRE sequence are preferably included. However the requirement for rev and RRE may be reduced or eliminated by codon optimization. Further details of this strategy can be found in our WO 01/79518. Alternative sequences which perform the same function as the rev/RRE system are also known. For example, a functional analog of the rev/KRE system is found in the Mason Pfizer monkey virus. This is known as CTE and comprises an RRE-type sequence in the genome which is believed to interact with a factor in the infected cell. The cellular factor can be thought of as a rev analog. Thus, CTE may be used as an alternative to the rev/RRE system. Any other functional equivalents which are known or become available may be relevant to the invention. For example, it is also known that the Rex protein of HTLV-I can functionally replace the Rev protein of HIV-I. It is also known that Rev and Rex have similar effects to IRE-BP. Packaging Sequence.

As used herein, the term “Modified” means a changed state or structure of a molecule or cell of the invention. Molecules may be modified in many ways, including chemically, structurally, and functionally. Cells may be modified through the introduction of nucleic acids.

As used herein, the term “Modulating,” means mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, a “Naive T cell” refers to a T cell that is antigen-inexperienced. In some embodiments, an antigen-inexperienced T cell has encountered its cognate antigen in the thymus but not in the periphery. In some embodiments, naive T cells are precursors of memory cells. In some embodiments, naive T cells express both CD45RA and CCR7, but do not express CD45RO. In some embodiments, naive T cells may be characterized by expression of CD62L, CD27, CCR7, CD45RA, CD28, and CD127, and the absence of CD95 or CD45RO isoform. In some embodiments, naive T cells express CD62L, IL-7 receptor-a, IL-6 receptor, and CD132, but do not express CD25, CD44, CD69, or CD45RO. In some embodiments, naive T cells express CD45RA, CCR7, and CD62L and do not express CD95 or IL-2 receptor β. In some embodiments, surface expression levels of markers are assessed using flow cytometry.

Unless otherwise specified, a “Nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “Operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “Overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

As used herein, the term “Packaging signal” refers to interchangeably as “Packaging sequence” or “Psi” refers to the non-coding, cis-acting sequence required for encapsidation of retroviral or lentiviral RNA strands during viral particle formation. In HIV-1, this sequence has been mapped to loci extending from upstream of the major splice donor site (SD) to at least the gag start codon.

As used herein, the term “Packaging cell” or “Producer cell” refers to a cell which contains those elements necessary for production of infectious recombinant virus which are lacking or non-functional in the viral genome. Typically, such packaging cells contain one or more producer plasmids which are capable of expressing viral structural proteins (such as codon optimized gag-pol and env) but they typically do not contain a packaging signal.

In one embodiment the packaging/producer cells of the present invention produce retroviral or lentiviral vector particles that are integration defective, in which the viral genome of the particle cannot integrate into the target cell's genome through the retroviral or lentiviral integration mechanism. In this embodiment, the packaging/producer cell is defective in a gene or sequence essential for integration. For example, the cell may comprise a disabled integrase gene, a disabled primer binding site (PBS) or a disabled att site. Preferably the entire integrase gene, PBS or att site is absent from the packaging/producer cell.

In another embodiment the packaging/producer cells of the present invention producing retroviral or lentiviral vector particles which upon infection of a target cell, do not allow for reverse transcription of the RNA genome. In this embodiment, the cell is defective in a gene or sequence essential for reverse transcription. For example, the cell may comprise a disabled reverse transcriptase gene. Preferably the entire reverse transcriptase coding region is absent from the packaging/producer cell.

As used herein, the term “Packaging cell line” or “Producer cell line” refers to a cell line which is capable of producing recombinant retroviral particles, comprising a packaging cell line and a transfer vector construct comprising a packaging signal. In some embodiment, “Packaging cell line” is used in reference to cell lines that do not contain a packaging signal, but do stably or transiently express viral structural proteins and replication enzymes (e.g., gag, pol and env) which are necessary for the correct packaging of viral particles. Any suitable cell line can be employed to prepare packaging cells of the invention. Generally, the cells are mammalian cells.

The production of infectious viral particles and viral stock solutions may be carried out using conventional techniques. Methods of preparing viral stock solutions are known in the art. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

By using producer/packaging cell lines, it is possible to propagate and isolate quantities of retroviral or lentiviral vector particles (e.g. to prepare suitable titres of the retroviral or lentiviral vector particles) for subsequent transduction of, for example, a site of interest (such as adult brain tissue). Producer cell lines are usually better for large scale production of vector particles.

In some embodiments, the packaging cell lines are second generation packaging cell lines. In some embodiments, the packaging cell lines are third generation packaging cell lines. In the third generation cell lines, a further reduction in recombination may be achieved by changing the codons. This technique, based on the redundancy of the genetic code, aims to reduce homology between the separate constructs, for example between the regions of overlap in the gag-pol and env open reading frames.

The packaging cell lines are useful for providing the gene products necessary to encapsidate and provide a membrane protein for a high titre vector particle production. The packaging cell may be a cell cultured in vitro such as a tissue culture cell line. Suitable cell lines include but are not limited to mammalian cells such as murine fibroblast derived cell lines or human cell lines. In some embodiments, the packaging cell line is a human cell line.

In a particular embodiment, the cells used to produce the packaging cell line are human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In preferred embodiments, the packaging cells are 293 cells, 293T cells, or A549 cells.

Alternatively, the packaging cell may be a cell derived from the individual to be treated. The cell may be isolated from an individual and the packaging and vector components administered ex vivo followed by re-administration of the autologous packaging cells. In more detail, the packaging cell may be an in vivo packaging cell in the body of an individual to be treated or it may be a cell cultured in vitro such as a tissue culture cell line.

As used herein, the term “Packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. A retroviral/lentiviral transfer vector of the present disclosure can be introduced into a packaging cell line, via transfection, transduction or infection, to generate a producer cell or cell line. The packaging vectors of the present disclosure can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, dihydrofolate reductase (DHFR), Gin synthetase or adenosine deaminase (ADA), followed by selection in the presence of the appropriate drug and isolation of clones. A selectable marker gene can be linked physically to genes encoding by the packaging vector, e.g., by IRES or self cleaving viral peptides.

As used herein, the term “Parenteral” administration of an immunogenic composition includes, e.g., subcutaneous (s.c.), intravenous (i.v.), intramuscular (i.m.), or intrasternal injection, or infusion techniques.

As used herein, the terms “Peptide,” “Polypeptide,” and “Protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, a “poly(A)” is a series of adenosines attached by polyadenylation to the mRNA. In some embodiments of a construct for transient expression, the poly(A) is between 50 and 5000. In some embodiments the poly (A) is greater than 64. In some embodiments the poly(A) is greater than 100. In some embodiments the poly(A) is greater than 300. In some embodiments the poly(A) is greater than 400. poly(A) sequences can be modified chemically or enzymatically to modulate mRNA functionality such as localization, stability or efficiency of translation.

As used herein, “Polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site. As used herein, “Transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the term “Polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the term “Promoter” is defined as a DNA sequence recognized by the synthetic machinery of the cell, or introduced synthetic machinery, required to initiate the specific transcription of a polynucleotide sequence.

As used herein, the term “Promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “Constitutive promoter” is a nucleotide sequence which, when operably linked with a polynucleotide which encodes or specifies a gene product, causes the gene product to be produced in a cell under most or all physiological conditions of the cell.

As used herein, the term “Inducible promoter” is a nucleotide sequence which, when operably linked with a polynucleotide which encodes or specifies a gene product, causes the gene product to be produced in a cell substantially only when an inducer which corresponds to the promoter is present in the cell.

As used herein, the term “Tissue-specific promoter” is a nucleotide sequence which, when operably linked with a polynucleotide encodes or specified by a gene, causes the gene product to be produced in a cell substantially only if the cell is a cell of the tissue type corresponding to the promoter.

As used herein, the term “Pseudotype” or “Pseudotyping” refers to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells. In a preferred embodiment of the invention, lentiviral envelope proteins are pseudotyped with VSV-G. In one embodiment, the invention provides packaging cells, which produce recombinant retrovirus, e.g., lentivirus, pseudotyped with the VSV-G envelope glycoprotein.

As used herein, the term “Recombinant viral vector” (RRV) refers to a vector with sufficient viral genetic information to allow packaging of an RNA genome, in the presence of packaging components, into a viral particle capable of infecting a target cell. The RRV carries non-viral coding sequences which are to be delivered by the vector to the target cell. A RRV is incapable of independent replication to produce infectious viral particles within the final target cell. Usually the RRV lacks a functional gag-pol and/or env gene and/or other genes essential for replication. The vector of the present invention may be configured as a split-intron vector. Preferably the RRV vector of the present dislosure has a minimal viral genome.

As used herein, the term “Replication-defective” or “Replication incompetent” refers to a virus that is not capable of complete and effective replication such that infective virions are not produced (e.g., replication-defective lentiviral progeny). The term “Replication-competent” refers to wild-type virus or mutant virus that is capable of replication, such that viral replication of the virus is capable of producing infective virions (e.g., replication-competent lentiviral progeny).

As used herein, the term “Replication incompetent” refers to a recombinant retrovirus that cannot replicate once it leaves the packaging cell.

As used herein, the term “Retroviral vector” refers to a viral vector or plasmid containing structural and functional genetic elements, or portions thereof, that are primarily derived from a retrovirus.

As used herein, the term “Self-inactivating vector” (SIN vector) refers to a replication-defective vector in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment of the invention, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also contemplated in the present disclosure. In some embodiments, the SIN vector is a retroviral or lentiviral vector.

In some embodiments, an additional safety enhancement is provided by replacing the U3 region of the 5′ LTR with a heterologous promoter to drive transcription of the viral genome during production of viral particles. The heterologous promoters may be selected from the group consisting of viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In some embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

As used herein, the term “Sendai virus” refers to a genus of the Paramyxoviridae family. Sendai viruses are negative, single stranded RNA viruses that do not integrate into the host genome or alter the genetic information of the host cell. Sendai viruses have an exceptionally broad host range and are not pathogenic to humans. Used as a recombinant viral vector, Sendai viruses are capable of transient but strong gene expression.

As used herein, the term “Signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

As used herein, the term “Single chain antibodies” refer to antibodies formed by recombinant DNA techniques in which immunoglobulin heavy and light chain fragments are linked to the Fv region via an engineered span of amino acids. Various methods of generating single chain antibodies are known.

As used herein, the term “Single-chain variable fragment” or “scFv” is a fusion protein of the variable regions of the heavy (VH) and light chains (VL) of an immunoglobulin (e.g., mouse or human) covalently linked to form a VH::VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., Tn-MUC1 binding domain, PSMA binding domain, or mesothelin binding domain) comprises an scFv having the configuration from N-terminus to C-terminus, VH—linker—VL. In some embodiments, the antigen binding domain (e.g., a Tn-MUC1 binding domain, a PSMA binding domain, or a mesothelin binding domain) comprises an scFv having the configuration from N-terminus to C-terminus, VL—linker—VH. Those of skill in the art would be able to select the appropriate configuration for use in the present invention.

The linker is typically rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010. Various linker sequences are known in the art, including, without limitation, glycine serine (GS) linkers such as (GS)n, (GSGGS)n, (GGGS)n, and (GGGGS)n, where n represents an integer of at least 1. Exemplary linker sequences can comprise amino acid sequences including, without limitation, GGSG (SEQ ID NO: 24), GGSGG (SEQ ID NO: 25), GSGSG (SEQ ID NO: 26), GSGGG (SEQ ID NO: 27), GGGSG (SEQ ID NO: 28), GSSSG (SEQ ID NO: 29), GGGGS (SEQ ID NO: 30), or GGGGSGGGGSGGGGS (SEQ ID NO: 31), and the like. Those of skill in the art would be able to select the appropriate linker sequence for use in the present invention. In one embodiment, an antigen binding domain (e.g., a Tn-MUC1 binding domain, a PSMA binding domain, or a mesothelin binding domain) of the present disclosure comprises a heavy chain variable region (VH) and a light chain variable region (VL). In some embodiments, the VH and VL is separated by the linker sequence having the amino acid sequence GGGGSGGGGSGGGGS (SEQ ID NO: 31). In some embodiments, the linker nucleic acid sequence comprises the nucleotide sequence GGTGGCGGTGGCTCGGGCGGTGGTGGGTCGGGTGGCGGCGGATCT (SEQ ID NO: 32).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences. Antagonistic scFvs having inhibitory activity have been described.

As used herein, the term “Specificity” refers to the ability to specifically bind (e.g., immunoreact with) a given target antigen (e.g., a human target antigen). A chimeric antigen receptor may be monospecific and contain one or more binding sites which specifically bind a target or a chimeric antigen receptor may be multi-specific and contain two or more binding sites which specifically bind the same or different targets. In certain embodiments, a chimeric antigen receptor is specific for two different (e.g., non-overlapping) portions of the same target. In certain embodiments, a chimeric antigen receptor is specific for more than one target.

As used herein, the term “Spacer domain” generally means any oligo- or polypeptide that functions to link the transmembrane domain to, either the extracellular domain or, the intracellular domain in the polypeptide chain. A spacer domain may comprise up to about 300 amino acids, e.g., about 10 to about 100 amino acids, or about 25 to about 50 amino acids.

As used herein, the term “Specifically binds,” with respect to an antibody, means an antibody or binding fragment thereof (e.g., scFv) which recognizes a specific antigen, but does not substantially recognize or bind other molecules in a sample. For example, an antibody that specifically binds to an antigen from one species may also bind to that antigen from one or more species. But such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “Specific binding” or “Specifically binding,” can be used in reference to the interaction of an antibody, a protein, a chimeric antigen receptor, or a peptide with a second chemical species, to mean that the interaction is dependent upon the presence of a particular structure (e.g., an antigenic determinant or epitope) on the chemical species; for example, a chimeric antigen receptor recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

As used herein, the term “Stimulation,” means a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, clonal expansion, and differentiation into distinct subsets.

As used herein, the term “Stimulatory molecule” means a molecule on a T cell that specifically binds with a cognate stimulatory ligand present on an antigen presenting cell.

As used herein, the term “Stimulatory ligand” means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, the terms “Subject” refers to a vertebrate. A vertebrate can be a mammal, such as a non-primate (e.g., cows, pigs, horses, cats, dogs, rats, etc.) or a primate (e.g., monkey and human). Mammals can include, without limitation, humans, non-human primates, wild animals, feral animals, farm animals, sport animals, and pets. In some embodiments, “Subject” and “Patient” are used interchangeably. Any living organism in which an immune response can be elicited may be a subject or patient. In certain exemplary embodiments, a subject is a human.

As used herein, the term “Substantially identical”, in the context of a nucleotide sequence, refers to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 75%, 80%, 85%, 90%, 91%, 92%, 93%, 94%, 95%, 96%, 97%, 98% or 99% identity to a reference sequence, for example, a sequence provided herein.

In some embodiments, the context of an amino acid sequence, the term “Substantially identical” refers to a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%. 91%, 92%, 93%, 94%, 95%, 96%, 97%, 98% or 99% identity to a reference sequence, for example, a sequence provided herein.

As used herein, the term “Substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

As used herein, the term “Target site” or “Target sequence” refers to a genomic nucleic acid sequence that defines a portion of a nucleic acid to which a binding molecule may specifically bind under conditions sufficient for binding to occur.

As used herein, the term “Targeting domain” used in connection with a gRNA, refers to a portion of the gRNA molecule that recognizes, or is complementary to, a target sequence. For example, a target sequence within the nucleic acid of a cell (e.g., within a gene).

As used herein, the term “Target sequence” refers to a sequence of nucleic acids complimentary, for example fully complementary, to a gRNA targeting domain. In some embodiments, the target sequence is disposed on genomic DNA. In some embodiment the target sequence is adjacent to (either on the same strand or on the complementary strand of DNA) a protospacer adjacent motif (PAM) sequence recognized by a protein having nuclease or other effector activity, e.g., a PAM sequence recognized by Cas9. In some embodiments, the target sequence is a target sequence of an allogeneic T cell target. In some embodiments, the target sequence is a target sequence of an inhibitory molecule. In some embodiments, the target sequence is a target sequence of a downstream effector of an inhibitory molecule.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, coupled to three dimeric modules CD3δ/CD3ε, CD3γ/CD3ε, and CD3ζ/CD3ζ. In some cells the TCR consists of gamma and delta (γ/δ) chains (CD3γ/CD3ε). In some embodiments, TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “Therapeutic” as used herein means a treatment and/or prophylaxis. A therapeutic effect is obtained by suppression, remission, or eradication of a disease state.

As used herein, the term “Therapy” refers to any protocol, method and/or agent (e.g., a CAR-T) that can be used in the prevention, management, treatment and/or amelioration of a disease or a symptom related thereto. In some embodiments, the terms “therapies” and “therapy” refer to a biological therapy (e.g., adoptive cell therapy), supportive therapy (e.g., lymphodepleting therapy), and/or other therapies useful in the prevention, management, treatment and/or amelioration of a disease or a symptom related thereto, known to one of skill in the art such as medical personnel.

As used herein, the term “Tracr” used in connection with a gRNA molecule, refers to a portion of the gRNA that binds to a nuclease or other effector molecule. In some embodiments, the tracr comprises nucleic acid sequence that binds specifically to Cas9. In some embodiments, the tracr comprises nucleic acid sequence that forms part of the flagpole. As used herein, the term “transfected” or “transformed” or “transduced” refers to a process by which an exogenous nucleic acid is transferred or introduced into a host cell. A “transfected” or “transformed” or “transduced” cell is one which has been transfected, transformed or transduced with an exogenous nucleic acid. The cell includes a primary subject cell and its progeny.

As used herein the term “Transduction” refers to the delivery of a gene(s) or other polynucleotide sequence using a retroviral or lentiviral vector by means of viral infection rather than by transfection. In some embodiments, the lentiviral vectors of the present disclosure are transduced into a cell through infection and provirus integration. In some embodiments, a target cell, e.g., a T cell, is “transduced” if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

As used herein, the terms “Treat,” “Treatment” and “Treating” refer to the reduction or amelioration of the progression, severity, frequency and/or duration of a disease or a symptom related thereto, resulting from the administration of one or more therapies (including, but not limited to, a CAR-T therapy directed to the treatment of solid tumors). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

As used herein, the term “Therapeutic” as used herein means a treatment. A therapeutic effect is obtained by reduction, suppression, remission, or eradication of a disease state. The term “prophylaxis” as used herein means the prevention of or protective treatment for a disease or disease state.

As used herein, the term “Under transcriptional control” or “Operatively linked” as used herein means that the promoter is in the correct location and orientation in relation to a polynucleotide to control the initiation of transcription by RNA polymerase and expression of the polynucleotide.

As used herein, the term a “Variant of any given sequence” is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally-occurring protein. In some embodiments, a “Variant” refers to a polypeptide that has a substantially identical amino acid sequence to a reference amino acid sequence, or is encoded by a substantially identical nucleotide sequence. In some embodiments, the variant is a functional variant. As used herein, the term “Functional variant” refers to a polypeptide that has a substantially identical amino acid sequence to a reference amino acid sequence, or is encoded by a substantially identical nucleotide sequence, and is capable of having one or more activities of the reference amino acid sequence.

As used herein, the term “Vector” refers to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In some embodiments, a vector is a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, viral vectors, Sendai viral vectors, adenoviral vectors, adeno-associated virus vectors, retroviral vectors, lentiviral vectors, plasmids (e.g., DNA plasmids or RNA plasmids), transposons, cosmids, bacterial artificial chromosomes, and variant viral vectors.

As used herein, the term “Vector particle production system” refers to a system comprising the necessary components for retroviral or lentiviral vector particle production.

As used herein, the term “Viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In some embodiments, the term viral vector may refer either to a virus or viral particle capable of transferring a nucleic acid into a cell or to the transferred nucleic acid itself. Viral vectors and transfer plasmids contain structural and/or functional genetic elements that are primarily derived from a virus.

As used herein, the term “Xenogeneic” refers to a graft derived from an animal of a different species.”

### EXAMPLES

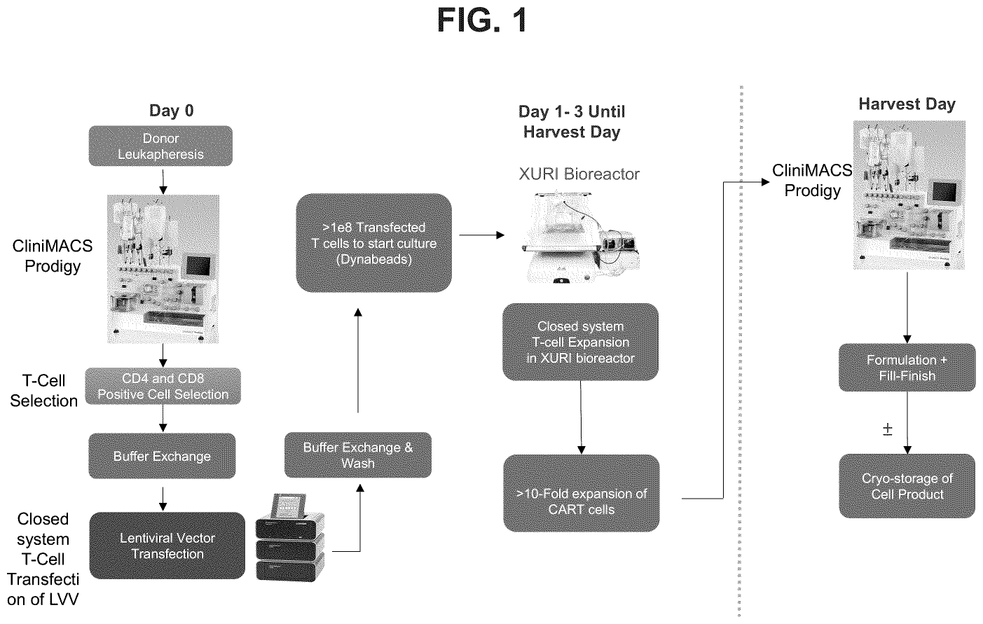

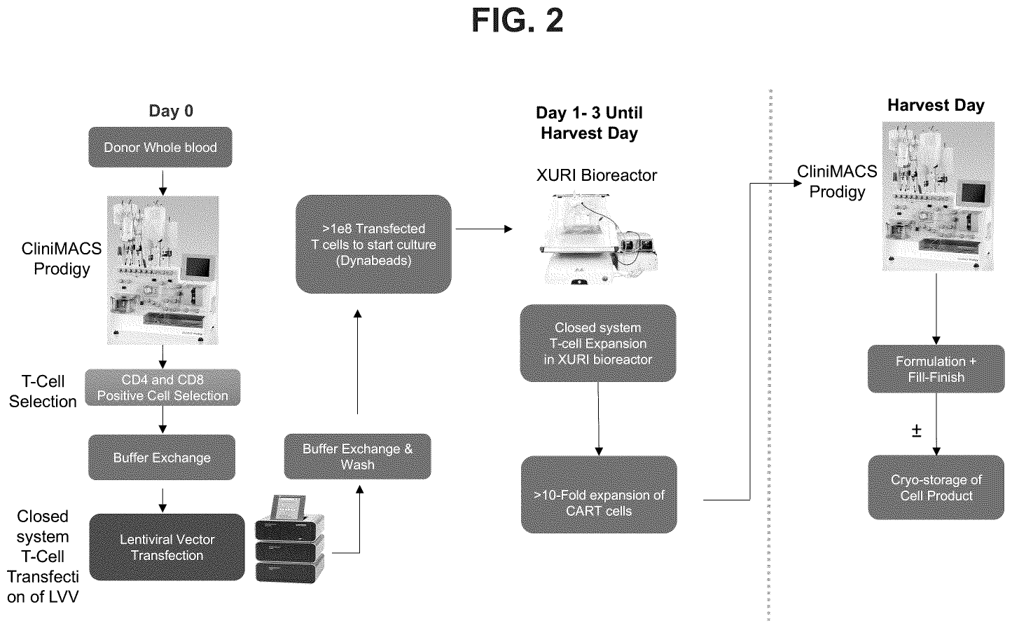

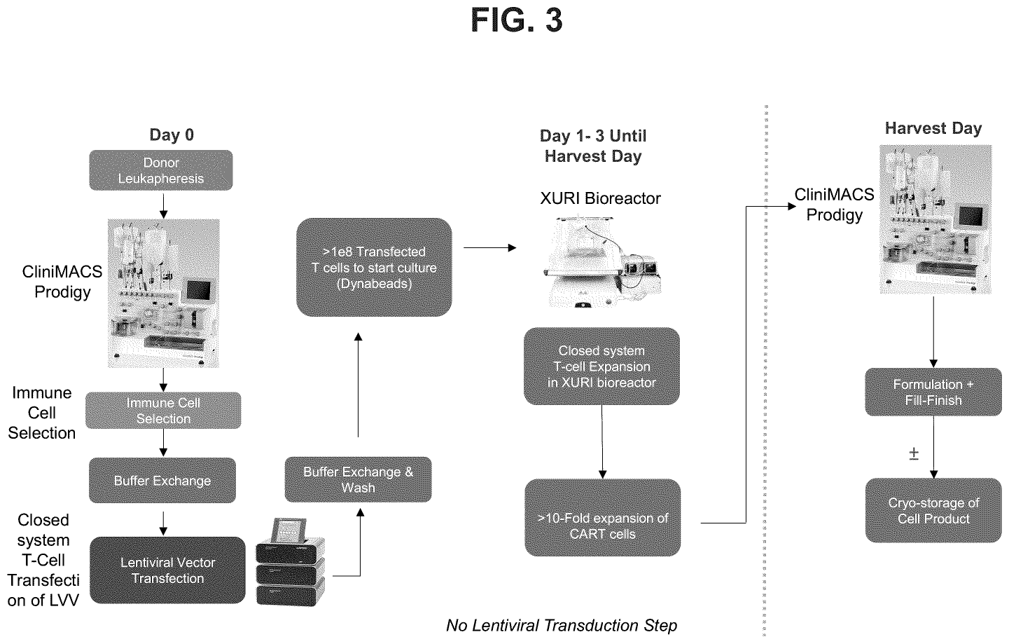

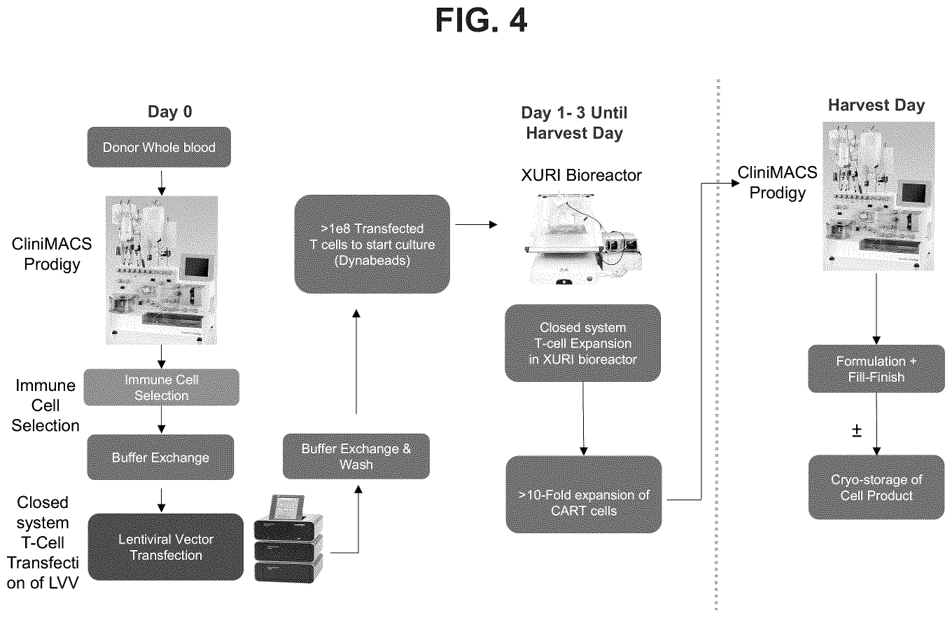

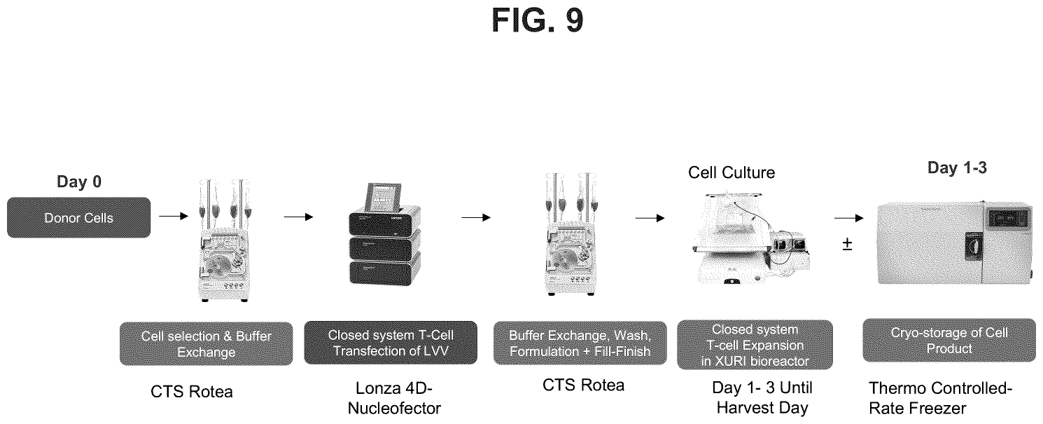

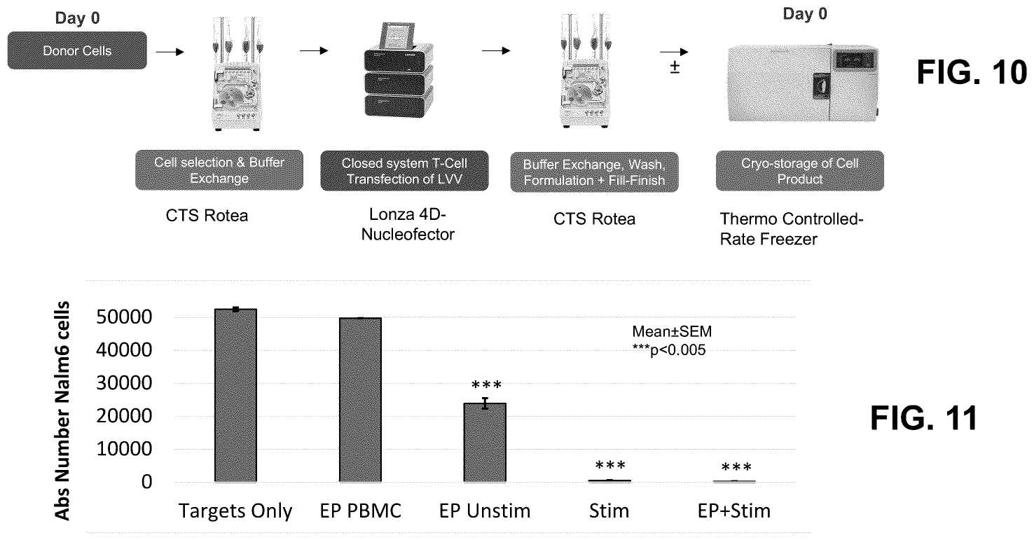

The following example describes novel manufacturing processes for engineering modified immune cells (e.g., T cells) for use in a method of treating diseases or disorders (e.g., cancer) described herein. Schematic illustrations of these processes are illustrated in FIGS. 1-10. FIGS. 1, 2, 4, 9, and 10 illustrate processes that require viral transduction. FIGS. 3, and 5-8 illustrate processes that require the electroporation of the viral vector into immune cells (e.g., T cells) rather than passive viral transfection. Furthermore, the processes described herein can start with either a Donor whole blood (FIGS. 2, 4, 6 and 8), a Donor leukapheresis product (FIGS. 1, 3, 5 and 7), or Donor cells (FIGS. 9 and 10). Prior to transfection, the Donor whole blood, or the Donor leukapheresis product may be enriched for immune cells generally (FIGS. 3, 4, 7, and 8) or CD4⁺ and CD8⁺ T cells (FIGS. 1, 2, 5, and 6).

### Example 1: Day 1-3 CAR T Cells Manufacturing Methods

T cell isolation. In this method, peripheral T cells or immune cells were separated from Donor whole blood using a conventional cell separation system at a clinical site (e.g., photophoresis or leukapheresis equipment). In some cases, the starting product was a donor Leukapheresis. This separation step comprised immune cells selection or a T cell selection to enrich for CD4⁺ and CD8⁺ T cells using a CliniMACS Prodigy® machine followed by one or more washing steps using Prodigy® process buffer (FIGS. 1-6). Immune cells were selected using Gibco™ CTS™ Rotea Counterflow Centrifugation System (FIGS. 7-10). To specifically enrich for T cells, the CliniMACS Prodigy® machine was used with the TS 520 tubing set and T Cell Transduction (TCT) program software version 1.0. The final enriched product was eluted in OpTmizer™ complete T cell medium. T cells or immune cells were purified and the purity of the cells was assessed by flow cytometry. The purified T cells or immune cells were cryopreserved in liquid nitrogen until required for use. However, it is preferred that purified cells (e.g., T cells or immune cells) be transfected with a lentiviral vector encoding a CAR, a TCR, or any modifying agent within 24 hours following the apheresis and enrichment. The optimal cells at the beginning of the cell culture was about 1×10⁸ cells. T cell concentration and viability were determined by AO/PI staining as enumerated by the Cellometer Vision (Nexcelom).

Culture initiation and transduction: The lentiviral transfection took place in a closed T-cell transfection of lentiviral vector system (e.g., Lonza 4D-Nucleofector). Purified cells were immediately seeded into a culture vessels. Within 24 hours following the apheresis or thawing, each vessel was seeded at a density of 0.5×10⁸ viable cells per cm² of membrane, plus GMP-grade TransAct, and brought to a final concentration of 1.0×10⁸ viable cells/mL with OpTmizer™ complete T cell media.

The media may contain Dynabeads® magnetic beads for example anti-CD3/anti-CD28 beads at a final bead to cell ratio of about 3:1 in the presence of one or more cytokines, such as Interleukin-2 (IL-2), Interleukin-3 (IL-3), Interleukin-6 (IL-6), Interleukin-7 (IL-7), Interleukin-7 receptor (IL-7R), Interleukin-11 (IL-11), Interleukin-12 (IL-12), Interleukin-15 (IL-15), Interleukin-15 receptor (IL-15R), Interleukin-18 (IL-18), Interleukin-18 receptor (IL-18R), Interleukin-21 (IL-21). However, the preferred cytokines are IL-2, IL-7, IL-6, IL-15, and/or IL-15Ra because it was observed that T cells expanded in the presence of these cytokines significantly increased T-cell proliferation and significantly decreased T cell apoptosis (e.g., by activating the Bcl-xl anti-apoptotic pathway).

Lentiviral vectors were then thawed at room temperature and added to each transduced culture at a MOI of 0.08, 0.2, 0.40, 0.45, or 5.0 based on the approximated T cell titer. No lentiviral vector was added to control vessels. In some cases, rather than passively transducing enriched T cells, the lentiviral vector at the same MOI was electroporated into T cells (FIGS. 3, 5-8, and 11). Transfected cells were then washed using an exchange buffered and cultured in the presence of Dynabeads® magnetic beads at a final bead to cell ratio of about 3:1 in the presence of one or more cytokines as described above. Transfected cells can be seeded in a Closed system T-cell expansion bioreactor, such as Xuri™ Cell Expansion System W25—Girgin Ltd (or any GE Healthcare's WAVE Bioreactor™ technology) for a desired amount of time (more than 2 hours and up to 14 days) or a desired expansion fold is reached (e.g., 10 fold expansion of CART cells or CAR immune cells) (FIGS. 1-4, and 9). Once culture is initiated, the transfected cells are incubated at 37° C. and 5% CO2 until ready for harvest.

Harvest. Transduced culture cells can be harvested at 12 to 72 hours or more after culture initiation. Cells are harvested by swirling the vessel to gently resuspend the cells off the membrane, then the full culture volume is resuspended and transferred by serological pipette to a conical tube. A small aliquot is taken for a pre-wash count, viability determination, and flow staining. The remainder of each culture was washed twice in 50 mL-100 ml, resuspended, and a post-wash aliquot taken to examine counts and viability. The CliniMACS Prodigy may also be used to harvest CAR T cells.

### Example 2: Electric CAR T Cell Manufacturing Methods

The Electric CAR T cell manufacturing process was made possible by a new strategy of transducing immune cells with a lentiviral vector comprising a nucleic acid encoding a CAR, a TCR and/or a polypeptide that enhanced the immune cell function, or a functional derivative thereof.

The Electric CAR T cell manufacturing method relies on an hybrid transfection method that combines a biological transfection (virus based transduction) as described in Example 1 above and a physical transfection, such as electroporation (e.g., Nucleofection®). Specifically, lentiviral particles were electroporated into immune cells or T cells. The electroporation of lentiviral particles into cells sped up the viral transfection/transduction and permitted the 1-day manufacturing of CAR T cells (e.g., Electric CAR T cells) without post transfection culture and/or expansion. As shown in FIGS. 5-8, and 10, electroporated cells were harvested within hours. As discussed in Examples 3 and 4, and shown in FIG. 11 and Tables 4-6, Electric CAR T cells efficiently killed target cells as well as conventional CAR T cells.

Ultrafast Electric CAR T cells were generated using the lentiviral Nucleofection Workflow shown in FIG. 7 or FIG. 8. The starting material for the manufacturing process was either whole blood or leukapheresis. T cells were isolated from this starting material using techniques known in the art (e.g., magnetic labeling of T cells followed by magnetic separation). Isolated T-cells were then electroporated with lentiviral vectors encoding a CAR (e.g., CD19 CAR) using a Lonza 4D Nucleofactor™ system (lentiviral Nucleofection™). The lentivirus was benzonase-treated. Transfected T-cells (Electric CAR T cells) were rested at changing temperatures and then cryopreserved using a ThermoFisher™ Scientific Controlled-Rate Freezer. Electric CAR T cells were then placed in a cryo-storage until needed. The generation of Electric CAR T cells using this manufacturing process did not require cytokines and/or an ex vivo culture process.

The Electric CAR T cells were first assessed to determine whether the CAR transgene had been integrated into the genome by PCR using the woodchuck hepatitis virus posttranscriptional regulatory element (WPRE) sequence as the amplicon (size of about 1.5 kb). Four groups of manufactured CART cells were tested by PCR: (1) CART cells that were stimulated (e.g., with CD3/CD28Dynabeads®), and electroporated (e.g., Nucleofection) with the CAR lentiviral vector; (2) CAR T cells that were stimulated and comprised the CAR lentiviral vector, but no Nucleofection; (3) CAR T cells that were electroporated with the CAR lentiviral vector; and (4) CAR T cells that contained the CAR lentiviral vector in the absence of any stimulation or nucleofection. The PCR results showed that all four groups of cells expressed the WPRE nucleic acid sequence. Surprisingly, the two groups of cells that were electroporated showed higher PCR products that were substantially similar in intensity. These results showed that nucleofection of CAR lentiviral vector resulted in effective CAR transgene integration into the T-cell genome. Furthermore, the integration efficiency was substantially similar in Electric CAR T cells generated with or without t CD3/CD28 Dynabeads® stimulation.

The Electric CAR T cells were also evaluated to determine the lentiviral vector copie number that was integrated in each T cell. This analysis showed that a lentiviral vector copy number per cell of Electric CAR T cells was substantially similar or slightly higher than the vector copy number per cell obtained with conventional (e.g., transduced; TDN) CAR T cells. For example, the vector copy number per cell in Electric CD19 CAR T cells was about 1.00 to about 1.50 copies/cell, whereas the copy number per cell of conventional CD19 CAR T cells was about 1.00 to about 1.25 copies/cell.

To determine the rate of CAR transgene expression post nucleofection, a quantitative RTPCR was performed on Electric CD19 CAR T cells at 1, 2, and 4 hours after electroporation using the CAR WPRE sequence. The GAPDH gene was used as a control. The mean CAR WPRE count (CT; qRTPCR Cycle Threshold) was about 23.269 CT at 1 hour post electroporation, about 23.892 CT at 2 hours post electroporation, and about 22.393 CT at 4 hours post electroporation. The GAPDH CT was about 31.687 CT at 1 hour post electroporation, about 32.764 CT at 2 hours post electroporation, and about 29.645 CT at 4 hours post electroporation. T cells that were electroporated with an empty vector (no lentivirus) had a mean GAPDH CT of about 33.449 CT at 1 hour post electroporation, about 29.904 CT at 2 hours post electroporation, and about 29.135 CT at 4 hours post electroporation. These results showed that the mRNA encoding the CAR transgene was expressed within about 1 hour post nucleofection.

### Example 3: Electroporation of a Very Low MOI Lentiviral Vector into Primary Human T Cells Produced Significant Amounts of Electric CAR-T Cells

This example describes the efficiency of the hybrid lentiviral transfection system disclosed herein at generating Electric CAR T cells.

On Day 0, healthy human donor Peripheral blood mononuclear cells (PBMCs) were isolated from whole blood by Ficoll extraction, and T cells were isolated from PBMC by negative selection using the Pan T Cell Isolation Kit II (Miltenyi Biotec). Isolated T cells were either stimulated or left unstimulated prior to being electroporated with a lentivirus vector described in Example 1 comprising a nucleic acid molecule encoding a CD19 CAR. The cells were electroporated with 5 μl of lentivirus at 0.2 MOI, 2 μl of the lentivirus at 0.08 MOI, and 10 μl of lentivirus vector at 0.4 MOI. All at a MOI of less than 2.5 transfection unit per cell (Tu/cell). As shown in Table 2, electroporation of stimulated T cells with a lentivirus at very low MOI resulted in a significant percentages of CD19 CAR positive T cells (CAR⁺). In particular, electroporation of stimulated T cells with 2 μl of the lentivirus at 0.08 MOI generated 12.3% of CD19 CAR⁺ T cells when compared to unstained (0%) or untransduced T cells (0.13%). While electroporation of stimulated T cells with 5 μl of the lentivirus at 0.2 MOI generated 18.9% of CD19 CAR⁺ T cells when compared to unstained (0%) or unstranduced cells (0.13%).

TABLE 2

| | % CAR Positive T Cells |
|---|---|
| Unstained | 0 |
| Untransduced | 0.13 |
| (EP) + 2 ul LVV | 12.3 |
| MOI = 0.08 | |
| EP + 5 ul LVV | 18.9 |
| MOI = 0.2 | |

EP: Electroporation;
LVV: Lentiviral Vector;
Gated on Live T cells

Table 3 shows that electroporation of unstimulated T cells with a lentivirus at very low MOI resulted in significant percentages of CD19 CAR positive T cells (CAR⁺) five days post-electroporation. In particular, electroporation of unstimulated T cells with 2 μl of the lentivirus at 0.08 MOI generated 1.7% of CD19 CAR⁺ T cells when compared to unstained (0%) or unstranduced cells (0.5%). While electroporation of unstimulated T cells with 5 μl of the lentivirus at 0.2 MOI generated 3.4% of CD19 CAR⁺ T cells when compared to unstained (0%) or unstranduced cells (0.13%). Electroporation of unstimulated T cells with 10 μl of the lentivirus at 0.4 MOI generated 6.5% of CD19 CAR⁺ T cells when compared to unstained (0%) or untransduced cells (0.13%).

TABLE 3

| | CAR Positive Cells |
|---|---|
| Unstained | 0.0 |
| EP Control | 0.5 |
| (EP) + 2 ul LVV | 1.7 |
| MOI = 0.08 | |
| EP + 5 ul LVV | 3.4 |
| MOI = 0.2 | |
| EP + 10 ul LVV | 6.5 |
| MOI = 0.4 | |

EP: Electroporation;
LVV: Lentiviral Vector;
Gated on Live T cells;
Flow performed on Day 5 Post-EP

In addition, human T cells were nucleofected with lentiviral vector encoding an anti-CD19 CAR. The generated Electric CAR T cells were neither activated ex vivo nor expanded. 5-6 days post nucleofection, CAR expression was assessed by flow cytometry to determine the CAR expression using an anti-idiotype antibody that specifically recognizes the CD19-FMC63 binder (CAR19-FMC63 clone) and gated on live T cells. The lentiviral vectors were used at a MOI of about 0.4. Flow cytometry analysis showed that T cells that were electroporated with the CD19 lentiviral vector (NF+LVV) had 39.8% of CD19 CAR P T cells. T cells that did not receive the lentivirus and electroporation (No LVV and No NF) had 0.0% CD19 CAR positive CAR T cells and T cells that only received the lentiviral vector and no electroporation (LVV and No NF) had 3.5% CD19 CAR positive CAR T cells.

This example demonstrates that electroporation of a lentiviral vector in both unstimulated and stimulated primary human T cells at a very low MOI produced significant amounts of CAR-T cells. The electroporation rates disclosed herein were very high when compared to conventional methods which result in about 1-3% CAR expression in T cells. In addition, this system is cost effective because a batch of cells using conventional method can cost $1 million per batch, which is enough to treat about 8 patients. However, the methods disclosed herein will generate a CAR T cell batch that is sufficient to infuse about 20 patients. As such, the methods disclosed herein double or triple the number of patients with the same cost and significantly reduce the CAR T cell cost per patient.

### Example 4: The Majority of Electric CAR T Cells Maintained a Less Differentiated Phenotype

This example illustrates that Electric CAR T cells manufactured using the hybrid transfection system disclosed generated CAR T cells with a phenotype that is known to favor CART cell persistence and potency when administered to cancer patients.

To further characterize the phenotype of Electric CAR T cells, human T cells were nucleofected with lentiviral vector encoding an anti-CD19 CAR as described above. The expression of the chemokine receptor CCR7 (a molecule involved in the recirculation of lymphocytes to lymph nodes) and CD45RO was then assessed to determine the population of naïve T cells (CD45RO⁻ CCR7⁺), central memory T cells (CD45RO⁺ CCR7⁺), Effector memory T cells (CD45RO⁺ CCR7⁻), and terminal effector T cells (CD45RO⁻ CCR7⁻).

T cells that were not electroporated but contained the lentiviral vector (LVV only (no NF) had 51.1% naïve T cells, 18.7% central memory T cells, 21% Effector memory T cells, and 8.9% Terminal effector T cells. CD19 Electric CAR T cells generated with Nucleofection™ protocol 1 (NF+LVV(1)) had 58% naïve T cells, 17% central memory T cells, 19.2% Effector memory T cells, and 4.88% Terminal effector T cells. CD19 Electric CAR T cells generated with Nucleofection™ protocol 2 (NF+LVV(2)) had 49.4% naïve T cells, 29.2% central memory T cells, 18.1% Effector memory T cells, and 3.28% Terminal effector T cells. These cells were not stimulated with CD3/CD28 Dynabeads™

Tables 4 and 5show phenotypic analyses of Electric CAR T cells demonstrating that the majority of Electric CAR T cells maintained a less differentiated phenotype (Table 4 “naïve”) when compared to Electric CAR T cells stimulated with CD3/CD28 Dynabeeads® for 11 Days (Table 5). Protocol 1 and 2 represent different nucleofection codes as per manufacturer indications (Lonza, 4D Nucleofector). The data disclosed therein are representative mean values of two independent experiments (n=2) using normal donor T cells.

As shown in Table 4, the majority of Electric CAR T cells conserved a less differentiated phenotype in the absence of stimulation after electroporation. For example, in unstimulated Electric CAR T cells, the percentages of the naïve T cells population did not change between the control treatment (LVV only No nucleofection (No NF) and two experimental nucleofection protocols. The percentages of the naïve Electric CAR T cells population was about 50%.

TABLE 4

| | | Central | Effector | Terminal |
|---|---|---|---|---|
| | Naive | Memory | Memory | Effector |
| LVV Only (No NF) | 51.1 | 18.7 | 21 | 8.9 |
| NF + LVV (1) | 58 | 17.9 | 19.2 | 4.88 |
| NF + LVV (2) | 49.4 | 29.2 | 18.1 | 3.28 |

In contrast, when the Electric CAR T cells were stimulated with CD3/CD28 Dynabeads® for 11 days, the phenotype of the majority of Electric CAR T cells changed. Stimulated T cells that were not electroporated but contained the lentiviral vector (LVV only (no NF) had 2.14% naïve T cells, 9.86% central memory T cells, 72.9% Effector memory T cells, and 15.1% Terminal effector T cells. Stimulated CD19 Electric CART cells generated with Nucleofection™ protocol 1 (NF+LVV(1)) had 8.18% naïve T cells, 50.4% central memory T cells, 37.6% Effector memory T cells, and 3.84% Terminal effector T cells. Stimulated CD19 Electric CART cells generated with Nucleofection™ protocol 2(NF+LVV(2)) had 1.37% naïve T cells, 8.08% central memory T cells, 76.2% Effector memory T cells, and 14.3% Terminal effector T cells.

Thus, in contrast to Table 4, the majority of stimulated Electric CAR T cells had more differentiated phenotype (e.g., effector memory T cells) (Table 5). Protocol 1 and 2 represent different nucleofection codes as per manufacturer indications (Lonza, 4D Nucleofector). The experiments show representative mean values of two independent experiments (n=2) using normal donor T cells.

TABLE 5

| | | Central | Effector | Terminal |
|---|---|---|---|---|
| | Naive | Memory | Memory | Effector |
| LVV Only (No NF) | 2.14 | 9.86 | 72.9 | 15.1 |
| NF + LVV (1) | 8.18 | 50.4 | 37.6 | 3.84 |
| NF + LVV (2) | 1.37 | 8.08 | 76.2 | 14.3 |

Since unstimulated Electric CAR T cells were not activated ex vivo (e.g., different from conventional CART cells produced in a 7-12 day process), the final CART product generated by the method disclosed herein is a desirable improvement because these CAR T cells conserved a non-activated (i.e., less differentiated) phenotype. The non-activated phenotype is desirable because it is known to favor CART cell persistence and potency in cancer patients.

### Example 5: Electric CD19 CAR T Cells (CART19 Cells) were Highly Cytotoxic

This example illustrates that Electric CAR T cells manufactured using the hybrid transfection system disclosed herein were highly cytotoxic.

Electric CD19 CAR T cells were generated by the methods described in example 2, and were evaluated to determine the efficiency of electroporated CD19 CART cell killing in response to CD19-expressing target cells (NaLm6 cells) in vitro. CD19 CART cells were used fresh or thawed on day 0 and incubated overnight to recover. On day 1, the CD19 CART cells were incubated with either a CD19 expressing NaLm6-luciferase or NaLm6-S-luciferase target cells at E:T ratios ranging from 0 to 20. Loss of luciferase signal resulting from cell killing was measured using Bright-Glo substrate on Day 2 and specific lysis was calculated according to the following formula: Specific lysis (%)=100−(sample luminescence/average maximal luminescence)*100.

As shown in FIG. 11, lentiviral vector transfected CD19 CAR T cells (Electric CART19 Cells) efficiently killed target cells. Stimulated and electroporated CD19 CAR T cells (EP+Stim) were as efficient (100%) at killing cancer cells as canonically transduced CAR T cells (Stim). Unstimulated and electroporated CD19 CAR T cells also efficiently killed target cells (e.g., over 60% of the CD19 expressing cells were killed when compared to control target cells). Furthermore, whole electroporated PBMCs also show cytotoxic activity (about 15% reduction in the general population.)

To further determine the killing capacity of Electric CD19 CAR T cells, target tumor cells, such as GFP-expressing Nalm6 leukemia cell line (“targets”), were co-cultured with increasing amounts of Electric CART cells (i.e., “effectors”) for a period of 48 hours. The effector: target ratios included 10:1, 5:1, 2.5:1, 1.25:1, 0.62:1, and 0.31:1. To determine percentage cell killing by Electric CART cells, an eTox dye (red fluorescence) that bound to dead cells was added to the supernatant of the Electric CAR T cells-GFP NaLm6 co-cultures, and the mfi was determined as shown in Table 6.

Table 6 shows the tumor cell killing capacity of Electric CAR T cells and demonstrates that CD19-Targeting Electric CART cells effectively killed Nalm6 cells (CD19⁺) as well as conventional T cells at various effector:target ratio. Target tumor cells (GFP-expressing Nalm6 leukemia cell line; “targets”) were co-cultured with increasing amounts of CART cells (i.e., “effectors”) for a period of 48 hours. To determine the percentage cell killing by CART cells, the eTox dye was added to the supernatant of the cultures (red fluorescence). Untransduced T cells were activated T cells with no vector added, that were expanded for 9 days. Conventional CAR T were activated T cells transduced with CAR19 lentivirus and expanded for 9 days. Nucleofection and no LVV were Nucleofected T cells with no lentivirus. The data are representative data from 3 independent experiments using normal donor T cells showing mean values (n=3).

For untransduced T cells, the percentage of target cell killing was about 10% at E:T of 10:1; 5% at E:T of 5:1; 2% at E:T of 2.5:1; 0% at E:T of 1.25:1; 0% at E:T of 0.62:1; and 0% at E:T of 0.31:1. For electroporated T cells with no lentiviral vector (NF No LVV), the percentage of target cell killing was about 10% at E:T of 10:1; 5% at E:T of 5:1; 1% at E:T of 2.5:1; 0% at E:T of 1.25:1; 0% at E:T of 0.62:1; and 0% at E:T of 0.31:1. For conventional CART cells, the percentage of target cell killing was about 100% at E:T of 10:1; 100% at E:T of 5:1; 100% at E:T of 2.5:1; 80% at E:T of 1.25:1; 80% at E:T of 0.62:1; and 60% at E:T of 0.31:1.

TABLE 6

| | % Target Cell Killing by Effector:Target Ratio | | | | | | |
|---|---|---|---|---|---|---|---|
| | Targets Only | 10:1 | 5:1 | 2.5:1 | 1.25:1 | 0.62:1 | 0.31:1 |
| Untransduced | 0 | 10 | 5 | 2 | 0 | 0 | 0 |
| Conventional CART | 0 | 100 | 100 | 100 | 80 | 80 | 60 |
| NF No LVV | 0 | 10 | 5 | 1 | 0 | 0 | 0 |
| ELECTRIC CARs | 0 | 100 | 100 | 80 | 50 | 20 | 2 |

For Electric CAR T cells, the percentage of target cell killing was about 100% at E:T of 10:1; 100% at E:T of 5:1; 80% at E:T of 2.5:1; 50% at E:T of 1.25:1; 20% at E:T of 0.62:1; and 2% at E:T of 0.31:1.

Table 6 shows that Electric CAR T cells effectively killed target cells at E:T ratio as low as 0.62:1 (20% target cell killing) when compared to untransduced T cells (0%), conventional CAR T cells (80%); and T cells nucleofected with an empty vector (no LVV) (0%). Significantly, Electric CAR T cells manufactured with a 1-Day protocol and having a less differentiated phenotype were as effective as conventional T cells that took 9 days to manufacture at killing target cells at E:T of 5:1 (100% killing).

The novel manufacturing method described herein provided the most efficient CAR T cell immunotherapy known to date for several reasons. The Electric CAR T cell manufacturing process reduced the entire CAR T manufacturing process to a single day or at most 3-days if expansion (e.g., culturing) is desired. As such, the Electric CART cell manufacturing time is shortened from 12-15 days to about 1 day or at most 3 days. In addition, fewer cells were required. For example, traditional manufacturing processes require up to about 300 million cells, while the manufacturing process disclosed herein worked efficiently with about 3 million cells because the cells are general very fresh.

Lastly, the manufacturing process is cost effective because a batch of cells using conventional method can cost $1 million per batch, which is enough to treat about 8 patients. However, the methods disclosed herein will generate CAR T cell batches that are sufficient to infuse about 20 patients. As such, the methods disclosed herein double or triple the number of treated patients with the same cost and significantly reduce the CAR T cell cost per patient.

### * * * * * * * * * * * *

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the phrase “consisting essentially of” will be understood to include those elements specifically recited and those additional elements that do not materially affect the basic and novel characteristics of the claimed technology. The phrase “consisting of” excludes any element not specified.

Table 1 discloses nucleic acid and amino acid sequences used in the methods described herein.

TABLE 1

| SEQ ID | | |
|---|---|---|
| NO: | Description | Sequences |
| 1 | G [Vesicular | MKCLLYLAFLFIGVNCKFTIVFPHNQKGTWKNVPSN |
| | stomatitis Indiana | YHYCPSSSDLNWHNDLIGTALQVKMPKSHKAIQADG |
| | virus]. | WMCHASKWVTTCDFRWYGPKYITHSIRSFTPSVEQC |
| | Accession No | KESIEQTKQGTWLNPGFPPQSCGYATVIDAEAVIVQ |
| | QQL06413 | VTPHHVLVDEYTGEWVDSQFINGKCSNDICPTVHNS |
| | | TTWHSDYKVKGLCDSNLISTDITFFSEDGELSSLGKG |
| | | GTGFRSNHFAYETGDKACKMQYCRHWGVRLPSGV |
| | | WFEMDDKDLFAAARFPECPEGSSISAPSQTSVDVSLI |
| | | QDVERILDYSLCQETWSKIRAGLPISPVDLSYLAPKNP |
| | | GTGPAFTIINGTLKYFETRYIRVDIVAPILSRMVGMIS |
| | | GTTTERELWDDWAPYEDVEIGPNGVLRTSSGYKFPL |
| | | YMIGHGMLDSDLHLSSKAQVFEHPHIQDAASQLPDD |
| | | ETLFFGDTGLSKNPIELVEGWFSGWKSSIASFFFIIGLII |
| | | GLFLVLRVGIYLCIKLKHTKKRQIYTDIEMNRLGK |
| 2 | Vesicular | IASFFFIIGLIIGLFLVLRVGIHLC |
| | stomatitis Virus G | |
| | protein (VSV-G) | |
| | transmembrane | |
| | domain | |
| 3 | Vesicular | SSIASFFFIIGLIIGLFLVLRVGIHLC |
| | stomatitis Virus G | |
| | protein (VSV-G) | |
| | transmembrane | |
| | domain | |
| 4 | VSV-G TM | IASFFFIIGSSIGLALVLAGIHLC |
| | variant | |
| 5 | VSV-G TM | SSIASFFFIIGSSIGLALVLAGIHLC |
| | variant | |
| 6 | VSV-G TM | IASFFFIIGSSIGLFLVLAGIHLC |
| | variant | |
| 7 | VSV-G TM | IASFFFIIGSSIGLALVLRVGIHLC |
| | variant | |
| 8 | VSV-G TM | IASFFFIIGSSIGLALVLRAGIHLC |
| | variant | |
| 9 | VSV-G TM | SSIASFFFIIFLVLRVGIHLC |
| | variant-deletion | |
| 10 | VSV-G TM | SSIASFFFIIALIIALFLVLRVGIHLC |
| | variant | |
| 11 | VSV-G TM | SSIASFFFIILLIILLFLVLRVGIHLC |
| | variant | |
| 12 | VSV-G TM | SSIASFFFIIALIIGLFLVLRVGIHLC |
| | variant | |
| 13 | VSV-G TM | SSIASFFFIIGLIIALFLVLRVGIHLC |
| | variant | |
| 14 | VSV-G TM | IASFFFIIGSSIGLALVLRAGIHLC |
| | variant | |
| 15 | Influenza | ILSIYSTVASSLALAIMVAGLSLW |
| | hemagglutinin A | |
| | chain | |
| | Transmembrane | |
| | domain | |
| 16 | VAMP 2 | MMIILGVICAIILIIIIVYFST |
| | transmembrane | |
| | domain | |
| 17 | Syntaxin | IMIICCVILGIIIASTIGGIFG |
| | transmembrane | |
| | domain | |
| 18 | Influenza | QDLPGNDNSTATLCLGHHAVPNGTIVKTITDDQIEVT |
| | hemagglutinin A | NATELVQSSSTGKICNNPHKILDGRDCTLIDALLGDP |
| | chain | HCDVFQDETWDLFVERGNAFSSCYPYDVPDYASLRS |
| | HEMA_I75A2 | LVASSGTLEFITEGFTWTGVTQNGGSSACKRGPASGF |
| | Accession No | FSRLNWLTKSGSTYPVLNVTMPNNDNFDKLYIWGV |
| | P43257 | HHPSTNQEQTNLYVQASGRVTVSTRRSQQTIIPNIGSR |
| | | PWVRGQSGRISIYWTIVKPGDVLVINSNGNLIAPRGY |
| | | FKMRTGKSSIMRSDAPIDTCVSECITPNGSIPNDKPFQ |
| | | NVNKITYGACPKYVKQNSLKLATGMRNVPEKQTRG |
| | | LFGAIAGFIENGWEGMIDGWYGFRHQNSEGTGQAAD |
| | | LKSTQAAIDQINGKLNRVIKKTNEKFHQIEKEFSEVEG |
| | | RIQDLEKYVEDTKIDLWSYNADVLVALENQHTIDLT |
| | | DSEMNKLFEKTRRQLRENAEDMGNGCFKIYHKCDN |
| | | ACIESIRNGTYDHDIYRDEALNNRFQIKGVELKSGYK |
| | | DWILWISFAISCFLLCVVLLGFIMWACQRGNIRCNI |
| | | CI |
| 19 | Influenza | MEKIVLLFAIVSLVKSDQICIGYHANNSTEQVDTIME |
| | hemagglutinin A | KNVTVTHAQDILEKTHNGKLCDLDGVKPLILRDCSV |
| | chain | AGWLLGNPMCDEFINVPEWSYIVEKANPVNDLCYPG |
| | HA | DFNDYEELKHLLSRINHFEKIQIIPKSSWSSHEASLGV |
| | Accession No | SAACPYQGKSSFFRNVVWLIKKNSTYPTIKRSYNNTN |
| | ABQ09853.1 | QEDLLVMWGIHHPNDAAEQTKLYQNPTTYISVGTST |
| | | LNQRLVPRIATRSKVNGQSGRMEFFWTILKPNDAINF |
| | | ESNGNFIAPEYAYKIVKKGDSTIMKSELEYGNCNTKC |
| | | QTPMGAINSSMPFHNIHPLTIGECPKYVKSNRLVLAT |
| | | GLRNSPQRERRRKKRGLFGAIAGFIEGGWQGMVDG |
| | | WYGYHHSNEQGSGYAADKESTQKAIDGVTNKVNSII |
| | | DKMNTQFEAVGREFNNLERRIENLNKKMEDGFLDV |
| | | WTYNAELLVLMENERTLDFHDSNVKNLYDKVRLQL |
| | | RDNAKELGNGCFEFYHKCDNECMESVRNGTYDYPQ |
| | | YSEEARLKREEISGVKLESIGIYQILSIYSTVASSLALA |
| | | IMVAGLSLWMCSNGSLQCRICIKFVSSDD |
| 20 | Syntaxin1A, | MKDRTQELRT AKDSDDDDDV TVTVDRDRFM |
| | Accession no. | DEFFEQVEEI RGFIDKIAEN |
| | NM 053788 | VEEVKRKHSAILASPNPDEK TKEELEELMS |
| | | DIKKTANKVR SKLKSIEQSI EQEEGLNRSS |
| | | ADLRIRKTQHSTLSRKFVEV MSEYNATQSD |
| | | YRERCKGRIQ RQLEITGRTT TSEELEDMLE |
| | | SGNPAIFASGIIMDSSISKQ ALSEIETRHS EIIKLENSIR |
| | | ELHDMFMDMA MLVESQGEMI |
| | | DRIEYNVEHAVDYVERAVSD TKKAVKYQSK |
| | | ARRKKIMIII CCVILGIIIA STIGGIFG |
| 21 | VAMP2, | MSATAATVPPAAPAGEGGPPAPPPNLTSNRRLQQTQ |
| | Accession No. | AQVDEVVDIMRVNVDKVLERDQKLSELDDRADALQ |
| | AAH55105 | AGASQFETSAAKLKRKYWW KNLKMMIILG |
| | | VICAIILIIIIVYFST |
| 22 | Cocal Envelope | MNFLLLTFIVLPLCSHAKFSIVFPQSQKGNWKNVPSS |
| | Amino | YHYCPSSSDQNWHNDLLGITMKVKMPKTHKAIQAD |
| | Acid | GWMCHAAKWITTCDFRWYGPKYITHSIHSIQPTSEQ |
| | | CKESIKQTKQGTWMSPGFPPQNCGYATVTDSVAVVV |
| | | QATPHHVLVDEYTGEWIDSQFPNGKCETEECETVHN |
| | | STVWYSDYKVTGLCDATLVDTEITFFSEDGKKESIGK |
| | | PNTGYRSNYFAYEKGDKVCKMNYCKHAGVRLPSGV |
| | | WFEFVDQDVYAAAKLPECPVGATISAPTQTSVDVSLI |
| | | LDVERILDYSLCQETWSKIRSKQPVSPVDLSYLAPKN |
| | | PGTGPAFTIINGTLKYFETRYIRIDIDNPIISKMVGKISG |
| | | SQTERELWTEWFPYEGVEIGPNGILKTPTGYKFPLFMI |
| | | GHGMLDSDLHKTSQAEVFEHPHLAEAPKQLPEEETL |
| | | FFGDTGISKNPVELIEGWFSSWKSTVVTFFFAIGVFILL |
| | | YVVARIVIAVRYRYQGSNNKRIYNDIEMSRFRK |
| 23 | Cocal Envelope | atgaacttcctcctgctgacttttatcgtgctgcctctctgctcccacgccaagttctcg |
| | Nucleic Acid | attgtgttcccccaatcccaaaaggggaactggaagaatgtgccctcctcgtaccac |
| | | tactgcccgtcctcctccgaccaaaactggcacaacgatctgctcggaatcaccatg |
| | | aaggtcaagatgcccaagacccataaggctattcaggccgacggctggatgtgcc |
| | | acgccgcgaagtggatcaccacctgtgacttccggtggtacggtccgaagtacatc |
| | | actcactcgattcactcaattcagccgactagcgagcagtgcaaagagagcatcaa |
| | | gcagacgaagcagggcacatggatgtcccccggattccctccccaaaactgcgga |
| | | tatgcgaccgtgaccgatagcgtggccgtggtggtgcaggccacccctcatcatgt |
| | | gcttgtggatgagtacaccggagaatggatcgacagccagttcccgaacggaaaat |
| | | gcgaaaccgaggagtgcgagactgtccacaactccactgtgtggtactccgactac |
| | | aaggtcacgggcttgtgcgacgcgactttggtggacaccgaaatcaccttctttagc |
| | | gaggatggaaagaaggagtccatcggcaaaccgaacactggttaccgctccaatt |
| | | acttcgcgtacgaaaagggagacaaagtctgcaagatgaattactgcaagcacgcc |
| | | ggtgtcaggctgccatcaggagtgtggttcgaattcgtggaccaggacgtgtacgct |
| | | gccgcgaagcttccggaatgtccagtcggggcaaccatttccgcaccgactcaga |
| | | cctctgtggatgtgtccctgatcctggacgtcgagagaatcctggactacagcctgt |
| | | gtcaggagacttggtcgaagattcgctccaagcagcccgtgtcacctgtggatctgt |
| | | cgtatctggccccgaagaaccctggtaccggcccagcctttaccatcataaacggg |
| | | accctgaagtacttcgaaactcggtatattcggattgacatcgacaaccccatcatct |
| | | cgaaaatggtcggaaagatcagcggatcccagacagaaagggaactctggaccg |
| | | aatggttcccgtacgagggcgtggaaatcggtccgaacgggatcctgaaaactcct |
| | | acgggctacaagttccccctcttcatgattgggcatggcatgctggactccgatctcc |
| | | acaagacctcccaagctgaagtgttcgagcaccctcacctggccgaagcacccaa |
| | | gcagctgccagaggaagaaaccctcttcttcggggacaccggaatctcgaagaac |
| | | ccggtggaactgattgagggctggttctcatcatggaagtccaccgtggtcaccttct |
| | | tcttcgccatcggagtgtttatcctgctttacgtggtggcccgcatcgtgattgccgtg |
| | | cggtacagataccagggctccaacaacaagcgcatctacaacgatatcgagatga |
| | | gccggttccgcaagtaa |
| 24 | linker | GGSG |
| 25 | linker | GGSGG |
| 26 | linker | GSGSG |
| 27 | linker | GSGGG |
| 28 | linker | GGGSG |
| 29 | linker | GSSSG |
| 30 | linker | GGGGS |
| 31 | linker | GGGGSGGGGSGGGGS |
| 32 | linker | GGTGGCGGTGGCTCGGGCGGTGGTGGGTCGGGTG |
| | | GCGGCGGATCT |
| 33 | CD8 alpha | IYIWAPLAGTCGVLLLSLVITLYC |
| | (CD8α) | |
| | transmembrane | |
| | domain amino | |
| | acid sequence | |
| 34 | CD8 alpha | ATCTACATCTGGGCGCCCTTGGCCGGGACTTGTGG |
| | (CD8α)transmem | GGTCCTTCTCCTGTCACTGGTTATCACCCTTTACTG |
| | brane domain | C |
| | nucleic acid | |
| | sequence | |
| 35 | CD8 alpha | TTTPAPRPPTPAPTIASQPLSLRPEACRPAAGGAVHTR |
| | (CD8α) hinge | GLDFACD |
| | domain amino | |
| | acid sequence | |
| 36 | CD8 alpha | ACCACGACGCCAGCGCCGCGACCACCAACACCGG |
| | (CD8α) hinge | CGCCCACCATCGCGTCGCAGCCCCTGTCCCTGCGC |
| | domain nucleic | CCAGAGGCGTGCCGGCCAGCGGCGGGGGGCGCAG |
| | acid sequence | TGCACACGAGGGGGCTGGACTTCGCCTGTGAT |
| 37 | CD28 | FWVLVVVGGVLACYSLLVTVAFIIFWV |
| | transmembrane | |
| | domain amino | |
| | acid sequence | |
| 38 | CD28 | TTTTGGGTGCTGGTGGTGGTTGGTGGAGTCCTGGC |
| | transmembrane | TTGCTATAGCTTGCTAGTAACAGTGGCCTTTATTAT |
| | domain nucleic | TTTCTGGGTG |
| | acid sequence | |
| 39 | hinge | DKTHT |
| 40 | hinge | CPPC |
| 41 | hinge | CPEPKSCDTPPPCPR |
| 42 | hinge | ELKTPLGDTTHT |
| 43 | hinge | KSCDKTHTCP |
| 44 | hinge | KCCVDCP |
| 45 | hinge | KYGPPCP |
| 46 | human IgG1 | EPKSCDKTHTCPPCP |
| | hinge | |
| 47 | human IgG2 | ERKCCVECPPCP |
| | hinge | |
| 48 | human IgG3 | ELKTPLGDTTHTCPRCP |
| | hinge | |
| 49 | human IgG4 | SPNMVPHAHHAQ |
| | hinge | |
| 50 | CD3 zeta | RVKFSRSADAPAYQQGQNQLYNELNLGRREEYDVL |
| | intracellular | DKRRGRDPEMGGKPRRKNPQEGLYNELQKDKMAEA |
| | signaling domain | YSEIGMKGERRRGKGHDGLYQGLSTATKDTYDALH |
| | amino acid | MQALPPR |
| | sequence | |
| 51 | CD3 zeta | AGAGTGAAGTTCAGCAGGAGCGCAGACGCCCCCG |
| | intracellular | CGTACCAGCAGGGCCAGAACCAGCTCTATAACGA |
| | signaling domain | GCTCAATCTAGGACGAAGAGAGGAGTACGATGTTT |
| | nucleic acid | TGGACAAGAGACGTGGCCGGGACCCTGAGATGGG |
| | sequence | GGGAAAGCCGAGAAGGAAGAACCCTCAGGAAGGC |
| | | CTGTACAATGAACTGCAGAAAGATAAGATGGCGG |
| | | AGGCCTACAGTGAGATTGGGATGAAAGGCGAGCG |
| | | CCGGAGGGGCAAGGGGCACGATGGCCTTTACCAG |
| | | GGTCTCAGTACAGCCACCAAGGACACCTACGACGC |
| | | CCTTCACATGCAGGCCCTGCCCCCTCGC |
| 52 | CD3 zeta (Q14K) | RVKFSRSADAPAYKQGQNQLYNELNLGRREEYDVL |
| | intracellular | DKRRGRDPEMGGKPRRKNPQEGLYNELQKDKMAEA |
| | signaling domain | YSEIGMKGERRRGKGHDGLYQGLSTATKDTYDALH |
| | amino acid | MQALPPR |
| | sequence | |
| 53 | CD3 zeta (Q14K) | AGAGTGAAGTTCAGCAGGAGCGCAGACGCCCCCG |
| | intracellular | CGTACAAGCAGGGCCAGAACCAGCTCTATAACGA |
| | signaling domain | GCTCAATCTAGGACGAAGAGAGGAGTACGATGTTT |
| | nucleic acid | TGGACAAGAGACGTGGCCGGGACCCTGAGATGGG |
| | sequence | GGGAAAGCCGAGAAGGAAGAACCCTCAGGAAGGC |
| | | CTGTACAATGAACTGCAGAAAGATAAGATGGCGG |
| | | AGGCCTACAGTGAGATTGGGATGAAAGGCGAGCG |
| | | CCGGAGGGGCAAGGGGCACGATGGCCTTTACCAG |
| | | GGTCTCAGTACAGCCACCAAGGACACCTACGACGC |
| | | CCTTCACATGCAGGCCCTGCCCCCTCGC |
| 54 | 4-1BB | KRGRKKLLYIFKQPFMRPVQTTQEEDGCSCRFPEEEE |
| | costimulatory | GGCEL |
| | domain amino | |
| | acid sequence | |
| 55 | 4-1BB | AAACGGGGCAGAAAGAAACTCCTGTATATATTCAA |
| | costimulatory | ACAACCATTTATGAGACCAGTACAAACTACTCAAG |
| | domain nucleic | AGGAAGACGGCTGTAGCTGCCGATTTCCAGAAGA |
| | acid sequence #1 | AGAAGAAGGAGGATGTGAACTG |
| 56 | 4-1BB | AAACGGGGCAGAAAGAAACTCCTGTATATATTCAA |
| | costimulatory | ACAACCATTTATGAGACCAGTACAAACTACTCAAG |
| | domain nucleic | AGGAAGATGGCTGTAGCTGCCGATTTCCAGAAGAA |
| | acid sequence #2 | GAAGAAGGAGGATGTGAACTG |
| 57 | CD28 | RSKRSRLLHSDYMNMTPRRPGPTRKHYQPYAPPRDF |
| | costimulatory | AAYRS |
| | domain amino | |
| | acid sequence | |
| 58 | CD28 | AGGAGTAAGAGGAGCAGGCTCCTGCACAGTGACT |
| | costimulatory | ACATGAACATGACTCCCCGCCGCCCCGGGCCCACC |
| | domain nucleic | CGCAAGCATTACCAGCCCTATGCCCCACCACGCGA |
| | acid sequence | CTTCGCAGCCTATCGCTCC |
| 59 | CD28(YMFM) | RSKRSRLLHSDYMFMTPRRPGPTRKHYQPYAPPRDF |
| | costimulatory | AAYRS |
| | domain amino | |
| | acid sequence | |
| 60 | CD28(YMFM) | AGGAGTAAGAGGAGCAGGCTCCTGCACAGTGACT |
| | costimulatory | ACATGTTCATGACTCCCCGCCGCCCCGGGCCCACC |
| | domain nucleic | CGCAAGCATTACCAGCCCTATGCCCCACCACGCGA |
| | acid sequence | CTTCGCAGCCTATCGCTCC |
| 61 | ICOS | TKKKYSSSVHDPNGEYMFMRAVNTAKKSRLTDVTL |
| | costimulatory | |
| | domain amino | |
| | acid sequence | |
| 62 | ICOS | ACAAAAAAGAAGTATTCATCCAGTGTGCACGACCC |
| | costimulatory | TAACGGTGAATACATGTTCATGAGAGCAGTGAACA |
| | domain nucleic | CAGCCAAAAAATCCAGACTCACAGATGTGACCCTA |
| | acid sequence #1 | |
| 63 | ICOS | ACAAAAAAGAAGTATTCATCCAGTGTGCACGACCC |
| | costimulatory | TAACGGTGAATACATGTTCATGAGAGCAGTGAACA |
| | domain nucleic | CAGCCAAAAAATCTAGACTCACAGATGTGACCCTA |
| | acid sequence #2 | |
| 64 | ICOS(YMNM) | TKKKYSSSVHDPNGEYMNMRAVNTAKKSRLTDVTL |
| | costimulatory | |
| | domain amino | |
| | acid sequence | |
| 65 | ICOS(YMNM) | ACAAAAAAGAAGTATTCATCCAGTGTGCACGACCC |
| | costimulatory | TAACGGTGAATACATGAACATGAGAGCAGTGAAC |
| | domain nucleic | ACAGCCAAAAAATCCAGACTCACAGATGTGACCCT |
| | acid sequence | A |
| 66 | CD2 | TKRKKQRSRRNDEELETRAHRVATEERGRKPHQIPA |
| | costimulatory | STPQNPATSQHPPPPPGHRSQAPSHRPPPPGHRVQHQ |
| | domain amino | PQKRPPAPSGTQVHQQKGPPLPRPRVQPKPPHGAAE |
| | acid sequence | NSLSPSSN |
| 67 | CD2 | ACCAAAAGGAAAAAACAGAGGAGTCGGAGAAATG |
| | costimulatory | ATGAGGAGCTGGAGACAAGAGCCCACAGAGTAGC |
| | domain nucleic | TACTGAAGAAAGGGGCCGGAAGCCCCACCAAATT |
| | acid sequence | CCAGCTTCAACCCCTCAGAATCCAGCAACTTCCCA |
| | | ACATCCTCCTCCACCACCTGGTCATCGTTCCCAGGC |
| | | ACCTAGTCATCGTCCCCCGCCTCCTGGACACCGTG |
| | | TTCAGCACCAGCCTCAGAAGAGGCCTCCTGCTCCG |
| | | TCGGGCACACAAGTTCACCAGCAGAAAGGCCCGC |
| | | CCCTCCCCAGACCTCGAGTTCAGCCAAAACCTCCC |
| | | CATGGGGCAGCAGAAAACTCATTGTCCCCTTCCTC |
| | | TAAT |
| 68 | CD27 | QRRKYRSNKGESPVEPAEPCRYSCPREEEGSTIPIQED |
| | costimulatory | YRKPEPACSP |
| | domain amino | |
| | acid sequence | |
| 69 | CD27 | CAACGAAGGAAATATAGATCAAACAAAGGAGAAA |
| | costimulatory | GTCCTGTGGAGCCTGCAGAGCCTTGTCGTTACAGC |
| | domain nucleic | TGCCCCAGGGAGGAGGAGGGCAGCACCATCCCCA |
| | acid sequence | TCCAGGAGGATTACCGAAAACCGGAGCCTGCCTGC |
| | | TCCCCC |
| 70 | OX40 | ALYLLRRDQRLPPDAHKPPGGGSFRTPIQEEQADAHS |
| | costimulatory | TLAKI |
| | domain amino | |
| | acid sequence | |
| 71 | OX40 | GCCCTGTACCTGCTCCGCAGGGACCAGAGGCTGCC |
| | costimulatory | CCCCGATGCCCACAAGCCCCCTGGGGGAGGCAGTT |
| | domain nucleic | TCAGGACCCCCATCCAAGAGGAGCAGGCCGACGC |
| | acid sequence | CCACTCCACCCTGGCCAAGATC |

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

What is claimed is:

1. A method for manufacturing a population of engineered immune cells, the method comprising:
(a) enriching a population of lymphocytes, a population immune cells or a population of CD4⁺ and CD8⁺ cells from blood obtained from a subject;
(b) admixing the population of lymphocytes, the population of immune cells or the population of CD4⁺ and CD8⁺ cells with one or more buffer solutions; and
(c) transfecting population of lymphocytes, the population of immune cells, or the population of CD4⁺ and CD8⁺ cells with an effective dose of a modifying agent;
thereby generating a population of modified lymphocytes, a population of modified immune cells or a population of modified CD4⁺ and CD8⁺ cells;
wherein steps 1(a)-(c) take place within 24 hours.

2. The method of claim 1, wherein prior to the enriching of the population of immune cells or the population of CD4⁺ and CD8⁺ cells, the blood is separated into a plasma constituent, a mononuclear cell-containing layer, a platelet layer, and red blood cells by apheresis to produce an apheresis product selected from erythrocytapheresis, thrombapheresis, thrombocytapheresis, leukapheresis, stem cells, plasmapheresis, and plateletpheresis.

3. The method of claim 1, wherein the population of immune cells or the population of CD4⁺ and CD8⁺ cells is enriched by apheresis, elutriation or gradient centrifugation.

4. A method for manufacturing a population of engineered immune cells, the method comprising:
(a) enriching a population of lymphocytes, a population of immune cells or a population of CD4⁺ and CD8⁺ cells from a donor leukapheresis;
(b) admixing the population of lymphocytes, the population of immune cells or the population of CD4⁺ and CD8⁺ cells with one or more buffer solutions; and
(c) transfecting the population of lymphocytes, the population of immune cells or the population of CD4⁺ and CD8⁺ cells with an effective dose of a modifying agent, thereby generating a population of lymphocytes, a population of modified immune cells or a population of modified CD4⁺ and CD8⁺ cells,
wherein steps 1(a)-(c) take place within 24 hours.

5. A method for manufacturing a population of engineered eukaryotic cells, the method comprising:
(a) obtaining a population of eukaryotic donor cells from a subject;
(b) admixing the population of eukaryotic donor cells with one or more buffer solutions; and
(c) transfecting the population of eukaryotic donor cells with an effective dose of a modifying agent, thereby generating a population of modified eukaryotic donor cells,
wherein steps 1(a)-(c) take place the same day.

6. The method of claim 1, wherein prior to the transfecting step (c), the population of immune cells, the population of CD4⁺ and CD8⁺ cells, or the population of lymphocytes is stimulated and/or activated with one or more stimulating agents.

7. The method of claim 6, wherein the modifying agent is selected from the group consisting of a small molecule agent, a biologic agent, a therapeutic, a protein, a peptide, a protein therapeutic, a peptide therapeutic, a chimeric antigen receptor, a heterologous T cell receptor, a viral vector, a vector, a retroviral vector, a lentiviral vector, an adenoviral vector, and an adeno-associated viral vector.

8. The method of claim 1, wherein the population of immune cells, the population of CD4⁺ and CD8⁺ cells, or the population of lymphocytes is transfected with an effective dose of a lentiviral vector or a retroviral vector.

9. The method of claim 8, wherein the effective dose of the retroviral vector or lentiviral vector comprises a multiplicity of infection (MOI) of about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1.0, about 1.25, about 1.5, about 2.0, about 3.0, about 4.0, or about 5.0.

10. The method of claim 8, wherein the effective dose of the retroviral vector or lentiviral vector comprises:
(a) about 2 ul of the lentiviral vector at a MOI of about 0.08;
(b) about 5 ul of the lentiviral vector at a MOI of about 0.2; or
(c) about 10 ul of the lentiviral vector at a MOI of about 0.4.

11. The method of claim 1, wherein the population of immune cells is selected from the group consisting of mononuclear cells, Lymphocytes rich cells, B lymphocytes, T lymphocytes, CD4⁺ T lymphocytes, CD8⁺ T lymphocytes, dendritic cells, monocytes, natural killer (NK) cells, natural killer T (NKT) cells, T-regulatory cells, CD4⁺ T-helper cells, CD8⁺ cytotoxic T lymphocytes (CTLs), CD62L⁺ cells, CD27⁺ cells, CCR7⁺ cells, CD45RO⁻ cells, CD45RA⁺ cells, neutrophils, basophils, eosinophils, megakaryocytes, stem cells, hematopoietic stem cells (HSCs), hematopoietic progenitor cells (HPCs), CD34⁺ cells, CD34⁺ peripheral blood stem cells, lymphokine-activated killer cells (LAKs), tumor infiltrating lymphocytes (TILs), mesenchymal stem cells, mast cells, a monocyte, a macrophage, a neutrophil, a basophil, an eosinophil, a dendritic cell, a megakaryocyte, and combinations thereof.

12. The method of claim 1, wherein a concentration of the population of immune cells, the population of CD4⁺ and CD8⁺ cells, or the population lymphocytes is:
(a) at least about 0.7×10⁷, at least about 0.8×10⁷, at least about 0.9×10⁷, at least about 1×10⁷, at least about 2×10⁷, at least about 4×10⁷, at least about 6×10⁷, at least about 8×10⁷, at least about 1×10⁸, or at least about 5×10⁸ cells/mL;
(b) from about 0.5×10⁶ cells/mL to about 4×10⁶ cells/mL;
(c) from about 0.5×10⁶ cells/mL to about 1×10⁸ cells/mL; or
(d) from about 4.0×10⁶ cells/mL to about 1×10⁸ cells/mL.

13. The method of claim 1, wherein transfecting is:
(a) selected from the group consisting of viral transfection, transduction, non-viral transfection, and hybrid of viral and non-viral transfection;
(b) electroporation of a viral particle; or
(c) electroporation and viral transfection (transduction).

14. The method of claim 1, wherein:
(a) the population of modified immune cells, the population of modified CD4⁺ and CD8⁺ cells, or the population of lymphocytes is not activated with one or more stimulating agents following or before transfection; and
(b) the population of modified immune cells, the population of modified CD4⁺ and CD8⁺ cells, or the population of modified lymphocytes is not expanded ex vivo following transfection.

15. The method of claim 1 further comprising stimulating and activating the population of modified immune cells, the population of modified CD4⁺ and CD8⁺ cells, or the population of lymphocytes with one or more stimulating agents to produce a population of activated modified immune cells, a population of activated modified CD4⁺ and CD8⁺ cells, or a population of activated modified lymphocytes.

16. The method of claim 15 further comprising expanding the population of activated lymphocytes, the population of activated modified immune cells, or the population of activated modified CD4⁺ and CD8⁺ cells for a predetermined time to produce a population of engineered lymphocytes, a population of engineered immune cells, or a population of engineered CD4⁺ and CD8⁺ cells.

17. The method of claim 16, wherein the expanding step is performed:
(a) under shaking conditions or rotating conditions;
(b) in a closed system;
(c) using a serum-free culture medium; and/or
(d) in the presence of one or more stimulating agents.

18. The method of claim 16, wherein the population of activated modified immune cells, the population of activated modified CD4⁺ and CD8⁺ cells, or the population of activated modified lymphocytes are expanded for at least about 2-fold, at least about 3-fold, at least about 4-fold, at least about 5-fold, at least about 6-fold, at least about 7-fold, at least about 8 fold, at least about 10-fold, at least about 15-fold, at least about 20-fold, or at least about 25-fold.

19. The method of claim 1 further comprising harvesting the population of modified lymphocytes, the population of modified immune cells, or the population of modified CD4⁺ and CD8⁺ cells for cryopreservation or administration.

20. The method of claim 19, wherein:
(a) harvesting comprises selecting and enriching for the engineered lymphocytes, engineered immune cells, or engineered CD4⁺ and CD8⁺ cells; or
(b) harvesting further comprises formulating the engineered lymphocytes, the engineered immune cells, or the engineered CD4⁺ and CD8⁺ cells for cryopreservation or administration to a subject in need thereof.

21. The method of claim 16, wherein the predetermined time is:
(a) less than 15 hours, less than 20 hours, less than 23 hours, less than about 24 hours; less than about 30 hours; less than about 48 hours; less than about 72 hours; less than about 96 hours; or less than about 120 hours; or
(b) about 1 day, about 2 days, about 3 days, about 4 days, about 5 days, about 6 days, about 7 days, about 8 days, about 9 days, about 10 days, about 11 days, about 12 days, about 13 days, about 14 days, or more days.

22. The method of claim 1, wherein the time from enriching and/or obtaining the population of lymphocytes, the population of immune cells, or the population of CD4⁺ and CD8⁺ cell to harvesting the engineered immune cells, or the engineered CD4⁺ and CD8⁺ cells is:
(a) about 72 hours or less;
(b) from about 18 hours to about 72 hours, from about 18 hours to about 36 hours, from about 18 hours to about 24 hours, from about 24 hours to about 72 hours, from about 24 hours to about 36 hours, or from about 36 hours to about 72 hours;
(c) less than about 2 hours, less than about 3 hours, less than about 4 hours, less than about 5 hours, less than about 6 hours, less than about 7 hours, less than about 8 hours, less than about 9 hours, less than about 10 hours, less than about 11 hours, less than about 12 hours, less than about 13 hours, less than about 14 hours, less than about 15 hours, less than about 16 hours, less than about 17 hours, less than about 18 hours, less than about 19 hours, less than about 20 hours, less than about 21 hours, less than about 22 hours, or less than about 23 hours;
(d) about 1 day, about 2 days, about 3 days, about 4 days, about 5 days, about 6 days, about 7 days, about 8 days, about 9 days, about 10 days, about 11 days, about 12 days, about 13 days, about 14 days, or more days; or
(e) about 1 day, about 3 days, about 4 days, about 5 days, or about 6 days.

23. The method of claim 1, wherein the electroporating step is performed in a closed system, a semi-closed, and/or a functionally closed system.

24. The method of claim 6, wherein the one or more stimulating agents are:
(a) selected from the group consisting of agonistic antibodies, cytokines, recombinant costimulatory molecules, anti-CD3 antibodies or fragments thereof, anti-CD28 antibodies or fragments, small drug inhibitors, and/or a combination thereof; or
(b) cytokines selected from the group consisting of Interleukin-2 (IL-2), Interleukin-3 (IL-3), Interleukin-6 (IL-6), Interleukin-7 (IL-7), Interleukin-7 receptor (IL-7R), Interleukin-11 (IL-11), Interleukin-12 (IL-12), Interleukin-15 (IL-15), Interleukin-15 receptor (IL-15R), Interleukin-18 (IL-18), Interleukin-18 receptor (IL-18R), Interleukin-21 (IL-21), granulocyte macrophage colony stimulating factor, alpha, beta or gamma interferon, erythropoietin, and a combination thereof; or
(c) anti-CD3 and anti-CD28 antibodies or fragments thereof and one or more cytokines; or
(d) cytokines selected from IL-15 and IL-7; IL-7 and IL-21; IL-7 and IL-2; IL-15 and IL-2; IL-7, IL-15, and IL-21; IL-15 and IL-15Ra; or IL-7, IL-15 and IL-15Ra.

25. The method of claim 8, wherein the lentiviral vector or retroviral vector comprises a nucleic acid sequence encoding a chimeric antigen receptor (CAR), an engineered T cell receptor (TCR), and/or a nucleic acid sequence encoding a polypeptide that enhances the immune cell function, or a functional derivative thereof or produces a therapeutic protein.
